(19) United States
(12) Patent Application Publication
(10) Pub. No.: US 2020/0384124 A1
(43) Pub. Date: Dec. 10, 2020

(54) VECTOR FOR PHARMACOLOGICALLY ACTIVE MATER-INSOLUBLE MOLECULES, AND PROCESS OF PREPARING SAME

(71) Applicants: UNIVERSITE DE BORDEAUX, BORDEAUX (FR); INSTITUT POLYTECHNIQUE DE BORDEAUX, TALENCE CEDEX (FR); CENTRE NATIONAL DE LA RECHERCHE SCIENTIFIQUE, PARIS CEDEX16 (FR); UNIVERSITE D'AIX-MARSEILLE, MARSEILLE (FR); ASSISTANCE PUBLIQUE-HOPITAUX DE MARSEILLE, MARSEILLE (FR); INSTITUT NATIONAL DE LA SANTE ET DE LA RECHERCHE MEDICALE-INSERM, PARIS (FR)

(72) Inventors: Mireille BLANCHARD-DESCE, BEGLES (FR); Diane BRAGUER, ALLAUCH (FR); Michel VAULTIER, CHATEAUGIRON (FR); Marie-Anne ESTEVE, MARSEILLE (FR); Jonathan DANIEL, AMBARES ET LAGRAVE (FR); Florian CORREARD, MARSEILLE (FR); Maeva MONTALEYTANG, MARSEILLE (FR)

(21) Appl. No.: 16/770,373

(22) PCT Filed: Dec. 5, 2018
(86) PCT No.: PCT/FR2018/053124

§ 371 Date: Jun. 5, 2020

(30) Foreign Application Priority Data
Dec. 5, 2017 (FR) 1761647

(51) Int. Cl. A61K 47/69; A61K 31/337; A61K 31/704; A61K 47/68; A61K 47/64; A61K 47/54; A61P 35/00

(52) U.S. Cl. CPC A61K 47/6929; A61K 31/337; A61K 31/704; A61K 47/6803; A61P 35/00; A61K 47/6845; A61K 47/64; A61K 47/545; A61K 47/6849

(57) ABSTRACT

Disclosed is a new vector for pharmacologically active water-insoluble molecules. These nanovectors, which are in the form of carbon nanoplatforms, are capable of solubilising the active molecules while reducing the side effects of the treatments. Also disclosed are the processes for synthesizing these nanoplatforms, as well as to the use thereof as a drug, particularly in the treatment of brain tumors.

### BACKGROUND OF THE INVENTION

The present invention relates to new vectors of pharmacologically active water-insoluble molecules with reduced side effects.

The main disadvantage of conventional anticancer chemotherapy has a very strong impact on patients via numerous adverse effects. These side effects pose many problems in the daily life of patients and become the main limiting factor of treatment.

These toxicity problems arise from two causes:
a lack of selectivity of the active molecule, which provides a low therapeutic index,
the presence of toxic solvents used to dissolve non-water-soluble compounds.

Two options are available to reduce the side effects of the treatments:
better addressing of cancer cells, so as to increase the effectiveness of the treatment while reducing its toxicity,
better solubilization of non-water-soluble compounds thanks to non-toxic solvents or vectors.

The development of nanoparticles is beginning to gain in importance and allows solving at least one of the two problems, in particular the problem of solubility.

One of the most widely used anti-tumor molecules is paclitaxel from the taxane family. This molecule has a solubility of less than 1 mg/l. The pharmaceutical formulation of this compound contains excipients such as castor oil to allow its solubilization. These excipients are themselves toxic.

In recent years, many nano-vectors allowing efficient transport of paclitaxel have been developed via various vectors:

TABLE 1
Main means of nano-vectorization
of active anti-tumor molecules.

| | Covalent | |
|---|---|---|
| | bond | Soluble |
| Albumin | No | Yes |
| Micelle | No | No |
| Liposome | No | No |
| Emulsion by | No | No |
| oral route | | |
| Nanodiamond | Yes | No |

But these new vectors often meet only one of the two criteria. For example, the nano-vector from albumin solves the problem of low solubility of the active molecule. On the other hand, the bond between the active molecule and albumin is weak. It is therefore easy to cleave this bond, which implies a risk of salting out of the active molecule in all parts of the body and therefore significant side effects.

In the same way, the nano-vector from diamond makes it possible to obtain nano-vectors bound by covalent bond to the active molecule. However, these diamond nanoparticles are not soluble in water. The short- and long-term toxic effects of these hard carbon nanoparticles are not known.

### SUMMARY OF THE INVENTION

One of the aspects of the invention relates to a new nanoplatform capable of covalently binding a pharmacologically active molecule, in particular anti-tumor, and not water-soluble, while remaining soluble in water.

Another aspect of the invention relates to a new nanoplatform comprising a pharmacologically active molecule, in particular an antitumor molecule, which is capable of covalently binding a fluorophore having an emission wavelength allowing monitoring of the nanoplatform.

Another aspect of the invention relates to a new nanoplatform comprising a pharmacologically active molecule, in particular antitumor, and which is capable of covalently binding an addressing agent which makes it possible to improve the targeted transport of said pharmacologically active molecule, in particular antitumor, and thus limit side effects.

According to one aspect of the invention, the present invention relates to a pharmacologically active nanoplatform which consists of:
a nanoplatform (NPC₂) comprising or consisting of carbon, hydrogen, oxygen, and nitrogen,
a pharmacologically active molecule (M),
optionally a fluorophore (F), and
optionally an addressing agent (A),
   and in which
NPC₂ is bound by covalent bonds with M via a linker L,
   said NPC₂ nanoplatform having the following properties:
the solubility of NPC₂ in an aqueous medium is 25 to 500 g/l
the Young's modulus of NPC₂ is from 1 to 4 GPa,
the density of NPC₂ is 1 to 3,
the dry size of NPC₂ is 10 to 40 nm,
the hydrodynamic diameter of NPC₂ is 10 to 150 nm,
   NPC₂ is substantially amorphous, said NPC₂ nanoplatform having on the surface carboxylic and hydroxyl acid functions and a number of primary amine grafting functions of 0.7 to 7 mmol per gram of nanoplatform, said pharmacologically active molecule M having an aqueous solubility of less than 200 mg/l,
   said pharmacologically active nanoplatform having an aqueous medium solubility of 5 to 500 g/l,
   with the provisio that:
   NPC₂ is substantially free of lipids, nucleic acids, proteins and peptides.

The active nanoplatforms according to the present invention can contain any pharmacologically active molecules as long as they are not soluble in water but made soluble in water following their bond with the nanoplatform.

According to one aspect of the invention, the subject of the present invention is an active nanoplatform which consists of:
a nanoplatform (NPC₂) comprising or consisting of carbon, hydrogen, oxygen, and nitrogen,
an active antitumor molecule (M),
optionally a fluorophore (F), and
optionally an addressing agent (A),
   and in which
NPC₂ is bound by covalent bonds with M via a linker L,
   said NPC₂ nanoplatform having the following properties:
   the solubility of NPC₂ in an aqueous medium is 25 to 500 g/l
the Young's modulus of NPC₂ is from 1 to 4 GPa,
the density of NPC₂ is 1 to 3,
the dry size of NPC₂ is 10 to 40 nm,
the hydrodynamic diameter of NPC₂ is 10 to 150 nm,
NPC₂ is substantially amorphous
   said NPC₂ nanoplatform having a number of primary amine functions of surface grafting of 0.7 to 7 mmol per gram of nanoplatform,
   said active antitumor molecule M having a solubility in an aqueous medium of less than 200 mg/l,
   said active nanoplatform having a solubility in an aqueous medium of 5 to 500 g/l, with the proviso that:
   NPC₂ is substantially free of lipids, nucleic acids, proteins and peptides. According to another aspect of the invention, the subject of the present invention is an active nanoplatform consisting of:
   of a nanoplatform (NPC₂) comprising or consisting of carbon, hydrogen, oxygen, and nitrogen in the form of primary amino group (s) and primary alkylamino group (s) of 1 to 10 carbons, in particular of primary amino group (s) and of primary ethylamino group (s),
an active antitumor molecule (M),
optionally a fluorophore (F), and
optionally an addressing agent (A),
   and in which
NPC₂ is bound by covalent bonds with M, optionally via a linker L, said NPC₂ nanoplatform having the following properties:
the solubility of NPC₂ in an aqueous medium is 25 to 500 g/l
the Young's modulus of NPC₂ is from 1 to 4 GPa,
the density of NPC₂ is 1 to 3,
the dry size of NPC₂ is 10 to 40 nm,
the hydrodynamic diameter of NPC₂ is 10 to 150 nm,
NPC₂ is substantially amorphous
   said nanoplatform NPC₂ having a number of primary amine functions of surface grafting of 1.4 to 7 mmol per gram of nanoplatform, said active anti-tumor molecule M having a solubility in aqueous medium of less than 200 mg/l,
said active nanoplatform having a solubility in an aqueous medium of 5 to 500 g/l,
with the provisio that:
NPC₂ is substantially free of lipids, nucleic acids, proteins and peptides.

According to another aspect of the invention, the subject of the present invention is an active nanoplatform consisting of:
a nanoplatform (NPC₂) comprising or consisting of carbon, hydrogen, oxygen, and nitrogen in the form of primary amino group(s) and primary alkylamino group(s) of 1 to 10 carbons, in particular of primary amino group(s) and of primary ethylamino group(s),
an active antitumor molecule (M),
optionally a fluorophore (F), and
optionally an addressing agent (A),
   and in which
NPC₂ is bound by covalent bonds with M via a linker L,
   said NPC₂ nanoplatform having the following properties: the solubility of NPC₂ in an aqueous medium is 25 to 500 g/l
the Young's modulus of NPC₂ is from 1 to 4 GPa,
the density of NPC₂ is 1 to 3,
the dry size of NPC₂ is 10 to 40 nm,
the hydrodynamic diameter of NPC₂ is 10 to 150 nm,
NPC₂ is substantially amorphous
   said nanoplatform NPC₂ having a number of primary amine functions of surface grafting of 1.4 to 7 mmol per gram of nanoplatform, said active anti-tumor molecule M having a solubility in aqueous medium of less than 200 mg/l,
   said active nanoplatform having a solubility in an aqueous medium of 5 to 500 g/l,
   with the provisio that:
NPC₂ is substantially free of lipids, nucleic acids, proteins and peptides

According to another embodiment, the solubility in aqueous medium of said active anti-tumor molecule is less than or equal to 200 mg/l relative to the active anti-tumor molecule in the non-salt form,

According to another embodiment, the optional addressing agent (A) in the active nanoplatform can however be a protein or a peptide.

According to another aspect of the invention, the subject of the present invention is an active nanoplatform as described above in which:

NPC₂ is bound by covalent bonds with M via a linker L,
chosen from the compounds of formula XI,

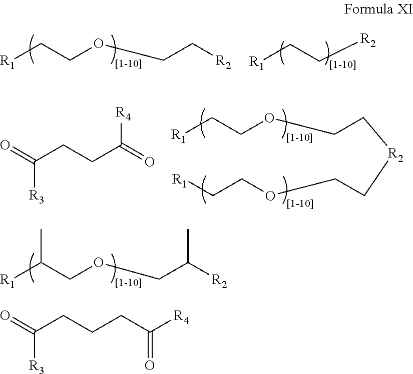

in which:
R₁ and R₂ are independently chosen from: —NH—, —COO, —NHCO—, —O—, —OCO—, —NHCSNH—, —NHCONH— and —CO—NH—NH—CO—
R₃ is chosen from: —NH—; —O—
R₄ is chosen from: —O—, —NHNH—, NH— in particular via a succinic linker.

For the purposes of the present invention, the term “nanoplatform” means a nanoparticle serving as a support and comprising surface grafting functions allowing the grafting of components to the surface, and including any fluorophore and addressing agent as long as the active antitumor molecule is not grafted.

Within the meaning of the present invention, the term “active nanoplatform” means a nanoplatform on which the active antitumor molecule is grafted, it being understood that the fluorophore and/or the addressing agent may or may not be present.

For the purposes of the present invention, the term “surface grafting functions” is understood to mean functions located on the surface of the nanoplatform capable of covalently binding the molecules of interest to the nanoplatform. These functions are chosen from preferably primary amine functions or carboxylic acid functions.

Within the meaning of the present invention, the term “molecules of interest” means the molecules to be grafted to the nanoplatform, i.e. either the active antitumor molecule, the fluorophore if it is present and/or the agent address if present.

For the purposes of the present invention, the term “substantially amorphous nanoplatform” is understood to mean a nanoplatform not comprising a characteristic line on the DRX spectrum, but capable of containing microcrystalline inclusions which are not detectable by this analysis method.

For the purposes of the present invention, the expression “nanoplatform substantially free of lipids, nucleic acids, proteins and peptides” means a nanoplatform containing less than 5% of lipids, less than 5% of nucleic acids, less than 5% of peptides and less than 5% of proteins by weight, compared to the total weight of the nanoplatform.

The active nanoplatforms according to the present invention have the advantage of being nanovectors of active antitumor molecules capable of dissolving said insoluble molecules thanks to the inherent solubility of the nanoplatform.

The active nanoplatforms according to the present invention have the advantage of having covalent bonds between the nanoplatform and the molecules attached to it. The interest is to be able to control the release or non-release of the molecules of interest attached to the active nanoplatform. Thus, it is possible, by choosing the covalent bonds appropriately, to allow the release of the active anti-tumor molecule only near or inside a tumor, while not at the same time releasing the fluorophore.

The nanoplatforms according to the present invention comprise:
either only an active anti-tumor molecule,
either an active anti-tumor molecule, and a fluorophore,
either an active anti-tumor molecule and an addressing agent,
or an active anti-tumor molecule, a addressing agent and a fluorophore.

The active nanoplatforms according to the present invention have the advantage of being modular. It is thus possible to graft different molecules according to the functions that one wishes to see fulfilled by the active nanoplatforms. Thus, if it is desired that the active anti-tumor molecule targets a particular type of cell, it is possible to attach an addressing agent to the active nanoplatform particularly addressing the cells concerned. But if we want the whole body to be targeted, the absence of an addressing agent makes it possible to remove the discriminating nature of the active nanoplatform.

The active nanoplatforms according to the present invention are soluble in an aqueous medium, which makes it possible to avoid adding toxic solvents, and are capable of efficiently transporting the active anti-tumor molecule to a destination targeted by the addressing agent (if present), and thus increase the selectivity of the active molecule. The nanoplatforms according to the present invention make it possible to overcome the two main causes of the side effects of the anti-tumor treatments known to date.

In a particular embodiment, the surface grafting functions of the active nanoplatform as described above, comprise NH₂ groups or consist of NH₂ groups, said surface grafting functions being capable of being bound by covalent bonds at M and/or F and/or A, the rate of bound grafting functions being comprised from approximately 50% to approximately 100% of the total of grafting functions at the surface of the NPC₂ nanoplatform.

Within the meaning of the present invention, the term “bound grafting function” means a grafting function on the surface of the nanoplatform onto which is grafted a compound which may be either a linker or a molecule of interest.

For the purposes of the present invention, the term “linker” is intended to mean a compound capable of covalently binding the nanoplatform and the molecules of interest. The linkers are identical or different depending on the molecules of interest.

For the purposes of the present invention, the term “non-water-soluble pharmacologically active molecule” means all of the pharmacologically active molecules whose solubility in aqueous medium is equal to or less than 200 mg per liter. Said solubility is relative to pharmacologically active molecules in their non-salt form. In the context of the present invention, the pharmacologically active molecules can however be used in the form of salts during the grafting process.

For the purposes of the present invention, the term “rate of bound grafting functions” means the ratio between the number of grafting functions actually bound and the number of grafting functions present at the surface.

In this embodiment, the molecules of interest which are bound to the nanoplatform, are bound on amine functions. These functions are the most reactive in the case of this invention. These functions bind more easily than the carboxylic acid functions.

Said amine functions are located on the surface of the nanoparticle as exemplified in the formula AAA below.

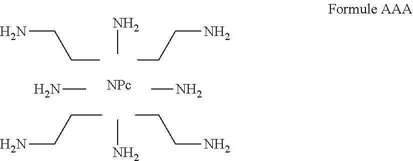

NP_{C} represents here the heart of the NPC₂ nanoplatform without the grafting functions located on the surface. The nanoplatform of the formula AAA carries “primary amine” functions (—NH₂), as well as “ethylamino” functions (—CH—CH₂—NH₂).

In a particular embodiment, the active nanoplatform as described above comprises a fluorophore F, and NPC₂ is bound by covalent bonds with F, via an L_{F} linker.

In this embodiment, the nanoplatform is bound with an active antitumor molecule and with a fluorophore. This fluorophore makes it possible to follow the evolution of the active nanoplatform to which it is bound in the organism, in particular in wavelengths adapted to biology.

In this embodiment, the covalent bonds make it possible to bond the nanoplatform with the fluorophore and to prevent the accumulation of fluorophore in the host organism. In a particular embodiment, the active nanoplatform as described above comprises an addressing agent A, and NPC₂ is bound by covalent bonds with A, via an L_{A} linker.

In this embodiment, the nanoplatform is bound with an active antitumor molecule and with an addressing agent. This addressing agent helps target cells or organs that need to be targeted for treatment. This addressing agent can be an antibody, a peptide or a small molecule which bind receptors strongly expressed by blood-brain barrier cells, tumor cells or which target neo-angiogenesis (LDLR, transferrin receptor, EGFR, VEGF). These may for example be RGD, TAT, angiopep-2 peptides, or anti EGFR or anti VEGF antibodies.

In a particular embodiment, the active nanoplatform as described above comprises a fluorophore F, and an addressing agent A, and NPC₂ is bound by covalent bonds with F via an L_{F} linker and NPC₂ is bound by covalent bonds with A via an L_{A} linker.

In this embodiment, the nanoplatform is bound with an active antitumor molecule, with an addressing agent and also with a fluorophore. This nanoplatform makes it possible to target the place of accumulation and action of the active nanoplatform thanks to the addressing agent, and to follow the evolution in the organism of the active nanoplatform thanks to the fluorophore, by fluorescence imaging.

In a particular embodiment, the active nanoplatform as described above comprises a fluorescent NPC₂ nanoplatform.

In this embodiment, the nanoplatform before its binding with the active antitumor molecule or even before the optional binding with the fluorophore is already fluorescent with an emission wavelength (for example, λmax=460 nm for the nanoplatform of example 1.1) unfavorable for imaging in a biological medium. It is for this reason that a fluorophore emitting at a wavelength favorable to the imaging of living things, typically the near infrared (700 nm<λmax<1000 nm), can be attached to the active fluorescent nanoplatform.

In a particular embodiment, the active nanoplatform as described above is of Formula I

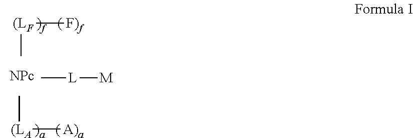

in which:
NP_{C} represents the core of the NPC₂ nanoplatform without the grafting functions located on the surface, NPC₂ having the above meaning,
M, F and A having the above meanings,
f and a are integers independently from 0 or 1,
L_{F}, L and L_{A} represent the linkers linking, via covalent bonds, the core of the NP_{C} nanoplatform with compounds F, M and A of Formula II, Formula IIa and Formula IIb,

T_{F}Z_{F}—R_{F}-Q_{F})_{I}_{_{F}} Formula II

TZ—R-Q)_{I} Formula IIa

T_{A}Z_{A}—R_{A}-Q_{A})_{I}_{_{A}} Formula IIb

in which:
T_{F}, T and T_{A} represent the grafting functions of the NPC₂ nanoplatform after their integration into L_{F}, L and L_{A} linkers,
Z_{F}, Z and Z_{A} represent the binding functions of the L_{F}, L and L_{A} linkers after their binding on the grafting functions T_{F}, T and T_{A},
R_{F}, R and R_{A} represent the functional chains of L_{F}, L and L_{A} linkers,
Q_{F}, Q and QA represent the binding functions of L_{F}, L and L_{A} linkers after their binding to the fluorophore, the active antitumor molecule and the addressing agent,
I, I_{F} and I_{A} are integers equal to or different from each other and are 0 or 1.

In this embodiment, the Lx linkers can take two types of organization:

a. either contain only the grafting function of the nanoplatform NPC_{Z}, which implies that the molecule of interest is bound directly to the nanoplatform, (Ix=0)
b. or contain, in addition to the grafting function of the NPC₂ nanoplatform, compound which is covalently bound to the previous grafting function. It is on this compound that the molecule of interest comes to bind (Ix=1).

The advantage of having a linker which is not composed solely of the grafting function of NPC₂ is that it can be used for the grafting of the active anti-tumor molecule by another covalent link. For example, if:
the active nanoplatform contains a fluorophore and an active antitumor molecule,
these two molecules of interest are bound on the same types of grafting functions, there is a risk that the fluorophore will be released under the same conditions as the active anti-tumor molecule. By adding/modifying the linker for one of the two molecules of interest, it is possible to have different grafting functions and different release conditions.

In this embodiment, the active nanoplatform can take 18 configurations:

TABLE 2
Table of possible configurations of Formula I

| Config- | Linker active | Fluoro- | Linker | Adressing | Linker |
|---|---|---|---|---|---|
| uration | molecule | phore | fluoro- | agent | Adressing |
| N° | L | F | phore L_{F} | A | agent L_{A} |
| 1 | Long | Present | Long | Present | Long |
| 2 | Long | Present | Long | Present | Short |
| 3 | Long | Present | Long | Absent | / |
| 4 | Long | Present | Short | Present | Long |
| 5 | Long | Present | Short | Present | Short |
| 6 | Long | Present | Short | Absent | / |
| 7 | Long | Absent | / | Present | Long |
| 8 | Long | Absent | / | Present | Short |
| 9 | Long | Absent | / | Absent | / |
| 10 | Short | Present | Long | Present | Long |
| 11 | Short | Present | Long | Present | Short |
| 12 | Short | Present | Long | Absent | / |
| 13 | Short | Present | Short | Present | Long |
| 14 | Short | Present | Short | Present | Short |
| 15 | Short | Present | Short | Absent | / |
| 16 | Short | Absent | / | Present | Long |
| 17 | Short | Absent | / | Present | Short |
| 18 | Short | Absent | / | Absent | / |

For the purposes of the present invention, the term “long linker” means a linker formed by the addition of a linker precursor and which does not only contain the grafting function derived from the nanoplatform. In this case, I, I_{F} and l_{A} are equal to 1.

For the purposes of the present invention, the term “short linker” means a linker formed solely by the grafting function originating from the nanoplatform. In this case, I, I_{F} and l_{A} are equal to 0.

For example, if you take Case 18 in Table 2, there is no fluorophore or addressing agent. This means that the linkers corresponding to these two molecules of interest are also absent. The linker for the active antitumor molecule is marked short. Case 18 therefore corresponds to the simplest case where only the active antitumor molecule is present.

In a particular embodiment, the active nanoplatform as described above is of Formula III,

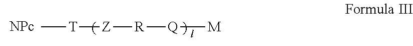

in which:
NP_{C}, T, Z, R, Q, M and I have the meanings set out above.

In this embodiment, the active nanoplatform follows two configurations from Table 2, configurations 9 and 18.

In this embodiment, the active nanoplatform contains only the active antitumor molecule, without fluorophore, and without addressing agent.

In a particular embodiment, the active nanoplatform as described above is of Formula IV,

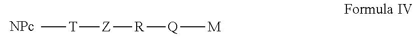

in which:
NP_{C}, T, Z, R, M and Q have the meanings set out above. In this embodiment, the active nanoplatform follows the configuration 9 in Table 2.

In this embodiment, the active nanoplatform comprises only an active molecule covalently bound to a nanoplatform via a long linker. This type of configuration can be advantageous in the case of an active molecule which is very slightly water-soluble. Indeed, it is possible to add a hydrophilic linker between the nanoplatform and the active molecule. This can help the solubilization of the active nanoplatform, in particular in the case where the grafting rate of the active anti-tumor molecule is high (close to 100%).

For the purposes of the present invention, the term “grafting rate of the active anti-tumor molecule” is understood to mean the ratio between the number of grafting functions bound to an active anti-tumor molecule and the number of grafting functions present on the surface of NPC₂.

In a particular embodiment, the active nanoplatform as described above has the formula V,

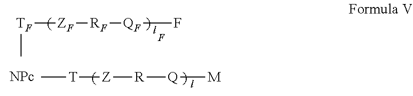

in which:
NP_{C}, T_{F}, T, Z_{F}, Z, R_{F}, R, Q_{F}, Q, I, F and M have the meanings stated above.

In this embodiment, the active nanoplatform follows the configurations 3, 6, 12 and 15 of Table 2.

In this embodiment, the active nanoplatform comprises an active antitumor molecule, and a fluorophore, without an addressing agent. The linkers are either in long configuration or in short configuration.

In a particular embodiment, the active nanoplatform as described above has the formula VI

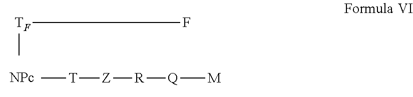

in which:
NP_{C}, T_{F}, T, Z, R, M, F and Q have the meanings set out above. In this embodiment, the active nanoplatform follows the configuration 6 of Table 2.

In this embodiment, the active nanoplatform comprises an active antitumor molecule, and a fluorophore, without an addressing agent. The active molecule is bound via a long linker, while the fluorophore is bound via a short linker to the nanoplatform.

In a particular embodiment, the active nanoplatform as described above has the formula VII,

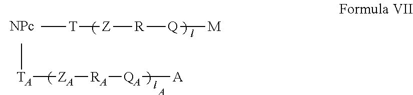

in which:
NP_{C}, T, T_{A}, Z, Z_{A}, R, R_{A}, Q, Q_{A}, I, I_{A}, A and M have the meanings stated above

In this embodiment, the active nanoplatform follows the configurations 7, 8, 16 and 17 of Table 2.

In this embodiment, the active nanoplatform comprises an active antitumor molecule, and an addressing agent, without fluorophore. The linkers are either in long configuration or in short configuration.

In a particular embodiment, the active nanoplatform as described above has the formula VIII

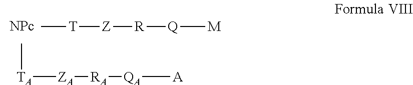

in which:
NP_{C}, T, T_{A}, Z, Z_{A}, R, R_{A}, Q, Q_{A} A and M have the meanings set out above.

In this embodiment, the active nanoplatform follows the configuration 7 of Table 2.

In this embodiment, the active nanoplatform comprises an active antitumor molecule, and an addressing agent, without fluorophore. The active molecule and the addressing agent are respectively bound via two long linkers, which may be different from each other, on the nanoplatform. In a particular embodiment, the active nanoplatform as described above is of Formula IX,

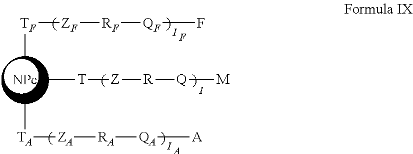

in which:
NP_{C}, T, T_{F}, T_{A}, Z, Z_{F}, Z_{A}, R, R_{F}, R_{A}, Q, Q_{F}, Q_{A}, I, I_{F}, I_{A}, F, A and M have the meanings stated above.

In this embodiment, the active nanoplatform follows the configurations 1, 2, 4, 5, 10, 11, 13 and 14 of Table 2.

In this embodiment, the active nanoplatform comprises an active antitumor molecule, a addressing agent, and a fluorophore. The linkers are either in long configuration or in short configuration, for each of the molecules of interest.

In a particular embodiment, the active nanoplatform as described above has the formula X

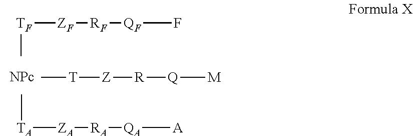

in which:
NP_{C}, T, T_{F}, T_{A}, Z, Z_{F}, Z_{A}, R, R_{F}, R_{A}, Q, Q_{F}, Q_{A}, F, M and A have the meanings set out above.

In this embodiment, the active nanoplatform follows configuration 1 of Table 2.

In this embodiment, the active nanoplatform comprises an active antitumor molecule, an addressing agent, and a fluorophore. The active molecule, the addressing agent and the fluorophore are respectively bound on the nanoplatform via long linkers, which may be different from each other. In general, the invention relates to an active nanoplatform in which said active antitumor molecule M is chosen from taxanes or anthracyclines. Among the taxanes, one can cite paclitaxel (known under the trade name of Taxol®) and docetaxel (known under the trade name of Taxotere®). Among the anthracyclines, one can cite epirubicin, pirarubicin, idarubicin, zorubicin, aclarubicin and in particular doxorubicin.

In particular, the invention relates to an active nanoplatform in which said active antitumor molecule M is chosen from taxanes such as paclitaxel or anthracyclines such as doxorubicin.

More particularly, the invention relates to an active nanoplatform in which said active antitumor molecule M is paclitaxel or doxorubicin.

Even more particularly, the invention relates to an active nanoplatform in which said active antitumor molecule M is paclitaxel.

Even more particularly, the invention relates to an active nanoplatform in which said active antitumor molecule M is doxorubicin.

In this embodiment, the active molecule being chosen from non-water-soluble active anti-tumor molecules, the active nanoplatform makes it possible to make said active anti-tumor molecules much more soluble in water, which improves their transport in the blood up to action areas. As a reminder of what is known, paclitaxel, an active molecule of the taxane family, has a solubility in water of the order of 0.5 mg/l. When paclitaxel is bound to the nanoplatform (example 3.1 below), the solubility of the corresponding active nanoplatform is at least 6.6 g/l.

In a particular embodiment, the content of active molecule of the active nanoplatform is from 10 to 150 mg per gram of active nanoplatforms, in particular from 30 to 100 mg/g, and more particularly 45 mg/g.

In a particular embodiment, the active nanoplatform as described above comprises a fluorophore F, chosen from Rhodamine B, Fluorescein isocyanate, Lucifer Yellow cadaverine, the family of Alexa Fluor or the family of cyanines NIR. The fluorophore can also be fluorescein.

In this embodiment, the fluorophore is chosen from a set of fluorophores which can be grafted onto the nanoplatform. The choice of fluorophore is made according to the emission spectrum of the chosen fluorophore, optionally see the absorption spectrum.

TABLE 3
Emission spectrum of the different
fluorophores that can be used.

| Fluorophore | Color | λₘₐₓ | |
|---|---|---|---|
| fluoresceine | Green | 494 | nm |
| Isothiocyanate | | | |
| Yellow Lucifer | Yellow | 536 | nm |
| Rhodamine B | Orange | 610 | nm |
| Alexa Fluo | Orange— | 612-782 | nm |
| | near infrared | | |
| Cyanines | Cyan— | 506-767 | nm |
| | near infrared | | |

In a particular embodiment, the active nanoplatform as described above comprises an addressing agent A, chosen from antibodies or vector peptides, in particular an antibody addressing EGF receptors, the RGD peptide or a peptide addressing LDLR. Addressing agent A can also be cetuximab.

In this embodiment, the addressing agent is chosen from a set of proteins capable of directing the active nanoplatform towards its target, and thus allowing the release of the active molecule on the preselected target.

In a particular embodiment, the active nanoplatform as described above comprises linkers L, L_{F} and LA chosen among the compounds of Formula XI.

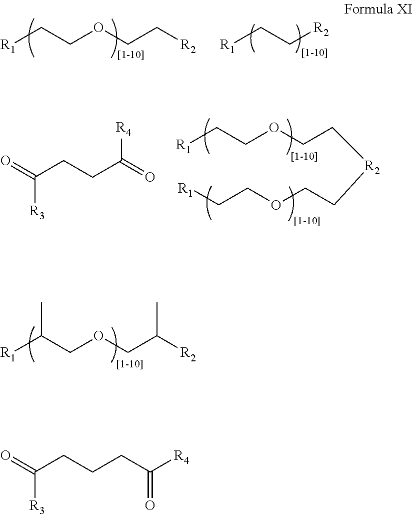

in which:
R₁ and R₂ are independently chosen from: —NH—, —COO, —NHCO—, —O—, —OCO—, —NHCSNH—, —NHCONH— and —CO—NH—NH—CO—
R₃ is chosen from: —NH—; —O—
R₄ is chosen from: —O—, —NHNH—, NH—
In particular a succinic linker

In this embodiment, the linkers are chosen according to several criteria:
the nature of the bonds to be formed with between the molecules of interest and the nanoplatform
their affinity with water and lipids,
the spatial congestion of the active molecule, to avoid steric congestion around the nanoplatform.

| (Gau)-G₁ | G₂-(Dro) | (Gau)-G₁G₂-(Dro) | Cleavage |
|---|---|---|---|
| —COOH | —NH₂ | —CONH— | possible |
| —NH₂ | —COOH | —NHCO— | possible |
| —NH₂ | —NHCOOH | —NHCONH— | difficult |
| —NH₂ | —NHCSOH | —NHCSNH— | difficult |
| —OH | —COOH | —OCO— | easy |
| —COOH | —OH | —COO— | easy |
| —OH | —OH | —O— | difficult/ |
| | | | photocleavable |
| —NH₂ | —OH | —NH— | difficult |
| —OH | —NH₂ | —NH— | difficult |

Table 4 describes a set of bonds that can serve as a link

Three configurations are possible:
either Gau represents NP_{C}, G₁ represents TF, G₂ represents ZF and Dro represents RF-QF-F.
either Gau represents NP_{C}, G₁ represents T, G₂ represents Z and Dro represents R-Q-M.
or Gau represents NP_{C}, G1 represents TA, G₂ represents Z_{A} and Dro represents R_{A}-Q_{A}-A.
   in which NP_{C}, T, T_{F}, T_{A}, Z, Z_{F}, Z_{A}, R, R_{F}, R_{A}, Q, Q_{F} and Q_{A} have the meanings set out in Formula 1.

The choice of the bonds which can covalently bond the molecule of interest to its own linker is made according to the choice of the bonds of table 4.

According to another aspect, the invention relates to a nanoplatform in which the bond between said active antitumor molecule M and either said linker L or said NPC₂ nanoplatform is cleavable under pH conditions from 2 to 8, in particular from 4 to 8 and preferably from 4 to 7.5. In other words, the pH conditions according to the invention in which the bond is cleavable are pH conditions of 2; 2.5; 3; 3.5; 4; 4.5; 5; 5.5; 6; 6.5; 7; 7.5 and/or 8.

In this particular embodiment, the bond between the active molecule and the nanoplatform is cleavable under the conditions chosen, which makes it possible to release the active molecule.

In this particular embodiment, it is possible to use a toxic active anti-tumor molecule, which is non-toxic when it is bound to the nanoplatform and which becomes toxic again, in particular for tumor cells, when it is released after cleavage. This embodiment makes it possible to limit the side effects of the active antitumor molecule, by limiting the toxicity of the active molecule during its transport.

In a particular embodiment, the active nanoplatform as described above is of Formula XII or of Formula XIIA,

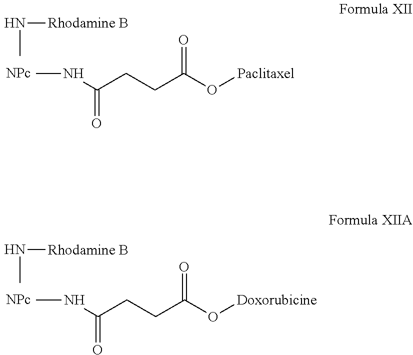

in which:
NP_{C} has the meaning described above.

In this particular embodiment, the nanoplatform is bound to a fluorophore (Rhodamine B), which makes it possible to follow the evolution of the nanoplatform by fluorescence in the field of orange. This fluorophore is directly bound to the nanoplatform via a short linker comprising only a primary amine grafting function. The nanoplatform is also bound to the active antitumor molecule, in this case paclitaxel, via a long linker comprising a primary amine grafting function and a compound terminated by a carboxylic acid function which, after binding with the active antitumor molecule, becomes an ester function.

In this particular embodiment, the active nanoplatform obtained has two different bonds, respectively for the fluorophore and for the active antitumor molecule. Thus, the conditions for cleavage of these two molecules of interest are different, which allows the release of one (the active antitumor molecule) without necessarily releasing the other (the fluorophore).

The present invention also relates to a process for the preparation of an active nanoplatform as described above comprising a step of grafting an active anti-tumor molecule:
by optionally bringing an NPC₂ nanoplatform into contact with a precursor L′ of said linker L, to obtain a nanoplatform bound by covalent bond to said linker L, followed
by bringing said nanoplatform bound by covalent bond to said linker L into contact with an active antitumor molecule M,
   in which NPC₂, L and M have the meanings of Formula I,
   to obtain an active nanoplatform consisting of a nanoplatform bound by covalent bond to said active antitumor molecule.

The synthesis method described above is the minimum required to obtain an active nanoplatform as described above. In this process, the linker can be:
either a short bond containing only the grafting function of the nanoplatform. This case is obtained by not adding a linker precursor.
or a long linker; this case is obtained by adding a linker precursor L′.

The synthesis method preferably provides, in the case where a linker precursor is present, to first bind this precursor on the nanoplatform before binding the active anti-tumor molecule.

In a particular embodiment, the preparation process as described above, comprises, before the step of grafting an active antitumor molecule, a step of binding a fluorophore:
by optionally bringing said NPC₂ nanoplatform into contact with an L′_{F} precursor of said L_{F} linker, to obtain a nanoplatform bound to said L_{F} linker, followed
by bringing said nanoplatform bound to said L_{F} linker into contact with a fluorophore F,
   in which NPC₂, L_{F} and F have the meanings of Formula I,
   to obtain a nanoplatform bound to said fluorophore.

In a particular embodiment, the preparation process as described above, comprises, before the grafting step of an active antitumor molecule, a step of refunctionalizing an initial nanoplatform By bringing an initial nanoplatform into contact with an organic molecule of the α-ω diamino-alkane type comprising two amine functions preferably two primary and/or secondary amine functions and even more preferably two primary amine functions to increase the rate of grafting functions comprising amine groups at the surface of said nanoplatform and thus obtaining a refunctionalized nanoplatform, followed

by an optional step of binding a fluorophore,
by optionally bringing said refunctionalized nanoplatform into contact with an L′_{F} precursor of said L_{F} linker, to obtain a refunctionalized nanoplatform bound to said L_{F} linker, followed
by optionally bringing said refunctionalized nanoplatform bound to said L_{F} linker into contact with a fluorophore F, in which NPC₂, L_{F} and F have the meanings of Formula I,
   to obtain a refunctionalized nanoplatform optionally bound to said fluorophore F.

In a particular embodiment, the organic molecule is an α-ω diamino-alkane of 1 to 10 carbon atoms, comprising two primary amine functions, in particular 1,2-ethylenediamine.

For the purposes of the present invention, the term “refunctionalization” means a step of transforming the grafting functions on the surface of the nanoplatform, which are not usable for grafting molecules of interest, in particular the acid, amide, or alcohols and convertible by reaction into sites carrying various functions which can be used for the abovementioned grafting and in particular a primary amine function.

In this particular embodiment, before the grafting step which makes it possible to graft the active antitumor molecule, the initial nanoplatform is refunctionalized to increase the rate of primary amine grafting functions on the surface of the nanoplatform.

On the surface of the initial nanoplatform, there are already amine grafting functions. It is therefore possible to use these grafting functions to graft the molecules of interest. It is also possible to use grafting functions different from the amine functions. The refunctionalization step is therefore only an optimization of the possible charge in terms of amine grafting functions of the nanoplatform.

In this particular embodiment, following the refunctionalization and before grafting the active molecule, it is possible to bond a fluorophore to the refunctionalized nanoplatform.

This possible fluorophore can be bound either to a short linker or to a long linker. In a particular embodiment, the preparation process as described above, comprises, after the step of grafting an active antitumor molecule, a step of binding an addressing agent:
by optionally bringing into contact of a nanoplatform bound by covalent bond to said active antitumor molecule M, with a precursor L′_{A} of said L_{A} linker, to obtain a nanoplatform bound to said active antitumor molecule M and bound to said L_{A} linker, followed
by contacting said nanoplatform bound by covalent bond to said active antitumor molecule M and bound to said L_{A} linker, with an addressing agent A,
   in which NPC₂, L_{A}, M and A have the meanings of Formula I,
   to obtain an active nanoplatform consisting of a nanoplatform bound by covalent bond to said active antitumor molecule M and bound to said addressing agent A.

In a particular embodiment, the preparation process as described above, comprises the following steps:
a. optionally a refunctionalization step of a nanoplatform,
   by bringing a nanoplatform into contact with an organic molecule of the α-ω diamino-alkane type comprising two amine functions preferably two primary and/or secondary amine functions and even more preferably two primary amine functions, in order to increase the rate of grafting functions comprising amine groups on the surface of said nanoplatform and thus obtaining an optionally re-functionalized nanoplatform,
b. optionally a step of binding a fluorophore,
   by optionally bringing said nanoplatform optionally re-functionalized into contact with precursor L′_{F} of said L_{F} linker, to obtain a nanoplatform optionally re-functionalized and optionally bound to said L_{F} linker, followed
   by optionally bringing said nanoplatform optionally re-functionalized and optionally bound to said linker L_{F} into contact with a fluorophore F, in order to obtain a nanoplatform optionally re-functionalized and optionally bound to said fluorophore F,
c. a step of grafting an active anti-tumor molecule,
   by optionally bringing said nanoplatform optionally re-functionalized and optionally bound to said fluorophore F into contact with a precursor L′ of said linker L, in order to obtain a nanoplatform optionally re-functionalized, optionally bound to said fluorophore F and bound by covalent bond to said linker L, followed
   by bringing said nanoplatform optionally re-functionalized, optionally bound to said fluorophore F and bound by covalent bond to said linker L into contact with an active antitumor molecule M, to obtain an active nanoplatform consisting of a nanoplatform bound by covalent bond to said active molecule antitumor M, optionally refunctionalized and optionally bound to said fluorophore F,
d. optionally a step of linking an addressing agent,
   by optionally bringing said nanoplatform bound by covalent bond to said active antitumor molecule M, optionally refunctionalized and optionally bound to said fluorophore F into contact with a precursor L′_{A} of said linker L_{A}, to obtain a nanoplatform bound by covalent bond to said active anti-tumor molecule M, optionally refunctionalized, optionally bound to said fluorophore F and optionally bound to said L_{A} linker, followed
   by optionally bringing said nanoplatform bound by covalent bond to said active antitumor molecule M, optionally refunctionalized, optionally bound to said fluorophore F and optionally bound to said L_{A} linker into contact with an addressing agent A,
      in which NPC₂, L_{F}, L, L_{A}, F, M and A have the meanings set out above,
      to obtain an active nanoplatform consisting of a nanoplatform bound by covalent bond to said active antitumor molecule M, optionally refunctionalized, optionally bound to said fluorophore F and optionally bound to said addressing agent A.

According to a particular embodiment, the optional addressing agent is a vector peptide.

According to a particular embodiment, the organic molecule is an α-ω diamino-alkane of 1 to 10 carbon atoms, comprising two primary amine functions, in particular 1,2-ethylenediamine.

According to yet another particular embodiment, the object of the present invention comprises a refunctionalization step.

According to another aspect of the invention, the subject of the present invention is a process for the preparation of an active nanoplatform as described above, comprising the following steps:
a. a step of re-functionalizing a nanoplatform,
   by bringing a nanoplatform into contact with an organic molecule of the α-ω diamino-alkane type of 1 to 10 carbon atoms, comprising two primary amine functions, in particular 1,2-ethylenediamine, in order to increase the rate of grafting comprising amine groups at the surface of said nanoplatform and thus obtaining a re-functionalized nanoplatform,
b. optionally a step of binding a fluorophore,
   by bringing said re-functionalized nanoplatform into contact with succinic anhydride to obtain a re-functionalized nanoplatform by means of a succinic linker, followed
   by bringing the nanoplatform refunctionalized by said succinic linker into contact with hydrazine to obtain a nanoplatform refunctionalized by a modified succinic linker, followed
   by optionally bringing said nanoplatform refunctionalized by a succinic linker modified into contact with a fluorophore F, the quantity of fluorophore F being substoichiometric with respect to the number of succinic linkers activated, to obtain a nanoplatform refunctionalized by an activated succinic linker and optionally bound to said fluorophore F,
c. a step of linking an addressing agent,
   by bringing said refunctionalized nanoplatform into contact with a modified succinic linker and optionally bound to said fluorophore F with an addressing agent A in order to obtain a refunctionalized nanoplatform optionally bound to said fluorophore F and bound to said addressing agent A,
d. a step of grafting an active anti-tumor molecule,
   by optionally bringing said refunctionalized nanoplatform, optionally bound to said fluorophore F and bound to said addressing agent A into contact with a precursor L′ of said linker L, in order to obtain a refunctionalized nanoplatform, optionally bound to said fluorophore F, bound to said agent addressing and optionally bound by covalent bond to said linker L, followed
   by bringing said refunctionalized nanoplatform into contact, optionally bound to said fluorophore F, bound to said addressing agent and optionally bound by covalent bond to said linker L with an active antitumor molecule M, to obtain an active nanoplatform consisting of a bound nanoplatform by covalent bond to said active anti-tumor molecule M, refunctionalized and optionally bound to said fluorophore F and bound to an addressing agent,
      in which NPC₂, L_{F}, L, L_{A}, F, M and A have the meanings stated above to obtain an active nanoplatform consisting of a nanoplatform bound by covalent bond to said active anti-tumor molecule M, refunctionalized, optionally bound to said fluorophore F and bound to said addressing agent A, said addressing agent being an antibody.

The synthesis method according to the present invention has the advantage of being modular. It is possible to modify:
the nature of the grafting functions, as well as their respective proportion,
the fluorescence of the active nanoplatform, in particular the color of this fluorescence,
the steric hindrance around the nanoplatform,
the nature of the covalent bonds linking the molecules of interest to the nanoplatform,
the grafting rate of molecules of interest on the nanoplatform,
the treatment addressing area.

In a particular embodiment, the process for the preparation as described above of said active nanoplatform of Formula I,

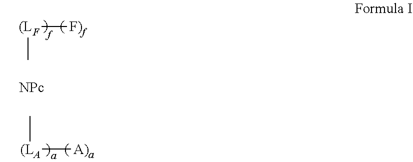

in which:
NP_{C}, F, M, A, L, L_{F}, L_{A}, a and f have the meanings stated above, comprises the following steps:
a. optionally a refunctionalization step by bringing into contact an NPC₁ nanoplatform of Formula a Formula a

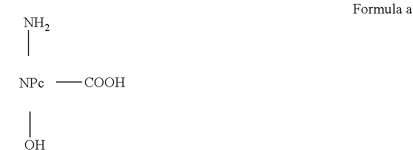

with an organic molecule of the α-ω diamino-alkane type comprising two amine functions, preferably two primary and/or secondary amine functions and even more preferably two primary amine functions, in order to obtain an NPC₂ nanoplatform of Formula b or of Formula B,

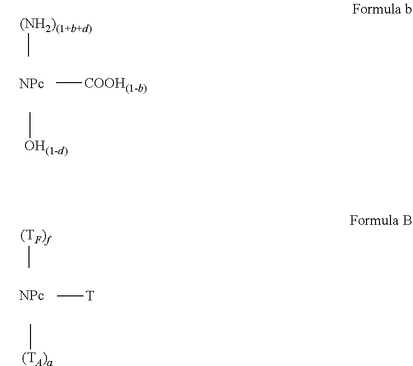

in which:
b and d are real numbers between 0 and 1 corresponding respectively to the rate of re-functionalization of the COOH grafting functions in NH₂ to the rate of re-functionalization of the OH grafting functions in NH2,
the grafting functions T_{F}, T and T_{A} are chosen from the grafting functions of the nanoplatform of Formula b (NH₂, COOH, OH),
a and f are whole numbers equal to 0 or 1, equal or different.
b. optionally a step of binding a fluorophore by optionally contacting an L′_{F} linker precursor of Formula 1

Z_{F}—R_{F}-Q_{F} Formula 1

with a NPC₂ nanoplatform of Formula B
to obtain a nanoplatform of Formula C

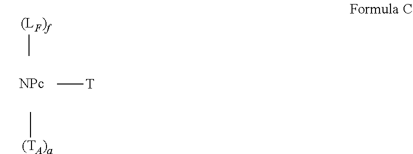

followed by bringing said nanoplatform of Formula C into contact with said fluorophore F to obtain a nanoplatform of Formula D,

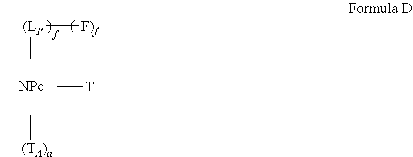

said step b taking place according to the following scheme

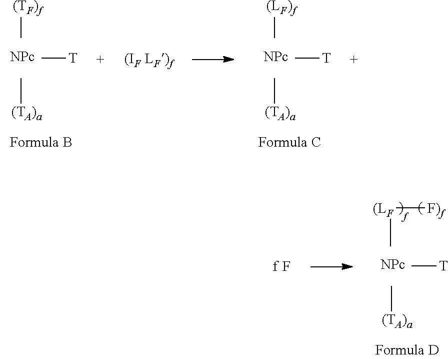

c. a step of grafting an active anti-tumor molecule by optionally contacting said nanoplatform of Formula D with a precursor of linker L′ of Formula 2

Z—R-Q Formula 2

to obtain a nanoplatform of Formula E,

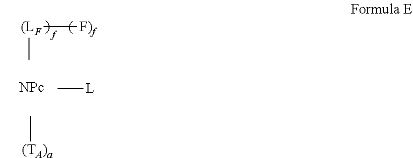

followed by bringing said nanoplatform of Formula E into contact with said active antitumor molecule M to obtain an active nanoplatform of Formula F,

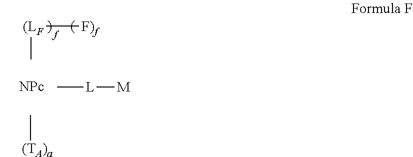

said step c taking place according to the following diagram

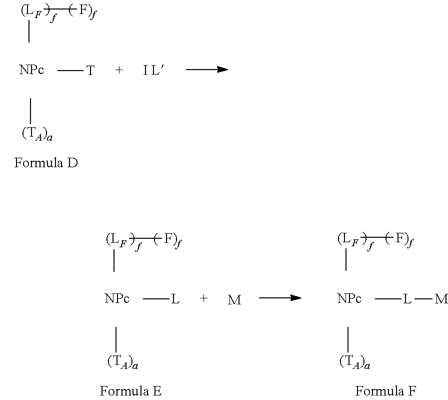

d. optionally a step of linking an addressing agent by optionally contacting said active nanoplatform of Formula F with a precursor of linker L′_{A} of Formula 3

Z_{A}—R_{A}-Q_{A} Formula 3

to obtain an active nanoplatform of Formula G

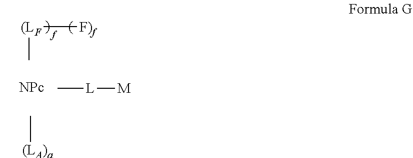

followed by bringing said active nanoplatform of Formula G into contact with said addressing agent A to obtain said active nanoplatform of Formula I,
said step d taking place according to the following diagram

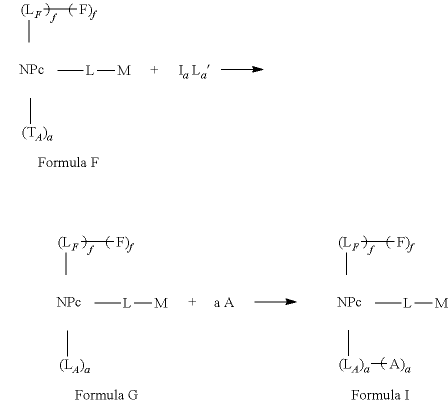

In this particular embodiment, the optional refunctionalization step is not total. If b=d=0 on Formula b, there is no re-functionalization and the grafting functions of the NPC₂ nanoplatform are the same as those of the initial NPC₁ nanoplatform. If b>0 or d>0, the refunctionalization is effective and unwanted grafting functions such as the OH and COOH functions react to obtain primary amine functions.

In this embodiment, Formula b and Formula B are two different scripts of the same NPC₂ nanoplatform. The grafting functions T, TA and TF are chosen from the grafting functions available on the surface of the nanoplatform of Formula b, ie NH2 and COOH.

In a particular embodiment, step a in the above method is
a. a refunctionalization step by bringing an NPC₁ nanoplatform of Formula a-b into contact

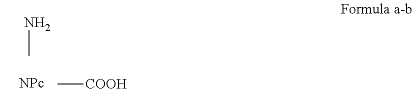

with an organic molecule of the type an organic molecule of the α-ω diamino-alkane type of 1 to 10 carbon atoms, comprising two primary amine functions, in particular 1,2-ethylenediamine, in order to obtain an NPC₂ nanoplatform of Formula b-b or of Formula B,

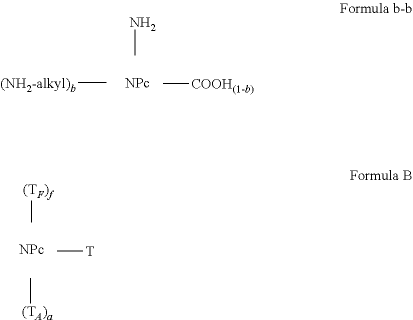

in which:
b is a real number from 0 to 1 corresponding to the rate of refunctionalization of the COOH grafting functions in NH₂
the grafting functions T_{F}, T and T_{A} are chosen from the grafting functions of the nanoplatform of Formula b-b (Alkyl-NH₂, NH₂, COOH),
a and f are integers from 0 or 1, equal or different,
with the proviso that the number b is greater than 0.

In this particular embodiment, the refunctionalization step is not total. If b=0 on Formula b, there is no re-functionalization and the grafting functions of the NPC₂ nanoplatform are the same as those of the initial NP_{C} nanoplatform. If b>0, the refunctionalization is effective and unwanted grafting functions such as the OH and COOH functions react to obtain primary amine functions.

In this embodiment, Formulas b-b and Formula B are two different scripts of the same NPC₂ nanoplatform. The grafting functions T, TA and TF are chosen from the grafting functions available on the surface of the nanoplatform of Formula b, ie NH₂ and COOH.

In this embodiment, a re-functionalization with 1,2-ethylenediamine leads to a structure of formula b-b in which the alkyl group is ethyl.

In this embodiment, an additional step of synthesis of the initial nanoplatform can be carried out.

In this embodiment, if a linker precursor is brought into contact with an active or inactive nanoplatform, the linker obtained following the binding of this precursor to the nanoplatform is a long linker. On the other hand, if a molecule of interest is bound without a linker precursor being bound, the linker obtained is a short linker.

In a particular embodiment, the process for the preparation as described above of said active nanoplatform of Formula IX,

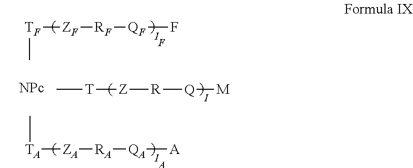

in which:
NP_{C}, T, T_{F}, T_{A}, M, F, A, Z, Z_{F}, Z_{A}, R, R_{F}, R_{A}, Q, Q_{F}, Q_{A}, I, I_{F} and I_{A} have the meanings of Formula I,
   comprises:
a. a refunctionalization step by bringing an NPC₁ nanoplatform of Formula a into contact,

### Formula a

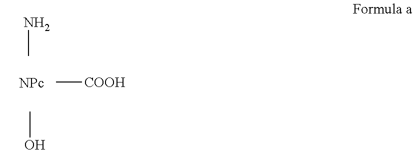

in which:
NPc having the meaning indicated in Formula I,
   with an organic molecule of type α-ω-diamino-alkane comprising two amine functions, preferably two primary and/or secondary amine functions and even more preferably two primary amine functions, in order to obtain an NPC₂ nanoplatform of Formula b or of Formula H,

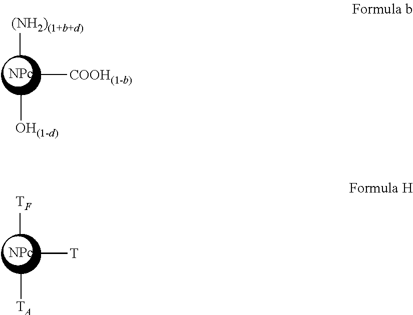

in which:
b and d are respectively the rate of re-functionalization of the COOH grafting functions in NH₂ and the rate of re-functionalization of the OFI grafting functions in NH₂, b and d being real numbers greater than 0,
the grafting functions T_{F}, T and T_{A} are chosen from the grafting functions of the nanoplatform of Formula b, in particular NH₂,
b. a step of binding a fluorophore by optionally bringing into contact a precursor of L′_{F} linker of Formula 1,

Z_{F}—R_{F}-Q_{F} Formula 1

with said NPC₂ nanoplatform of Formula H,
to obtain a nanoplatform of Formula J,

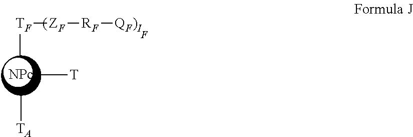

followed by bringing said nanoplatform of Formula J into contact with said fluorophore F,
to obtain a nanoplatform of Formula K,

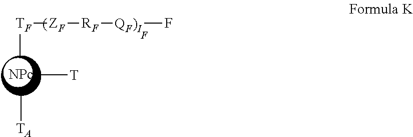

said step b taking place according to the following diagram

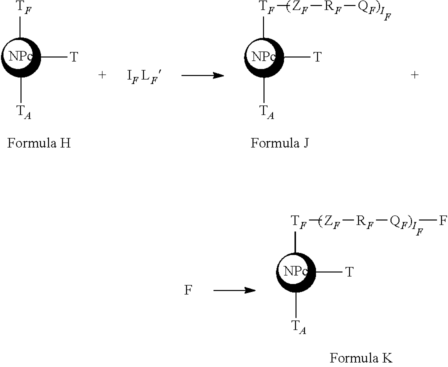

c. a step of grafting an active anti-tumor molecule by optionally contacting said nanoplatform of Formula K with a precursor of linker L′ of Formula 2

Z—R-Q Formula 2

to obtain a nanoplatform of Formula L

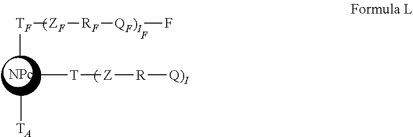

followed by bringing said nanoplatform of Formula L into contact with said active antitumor molecule M to obtain an active nanoplatform of Formula M,

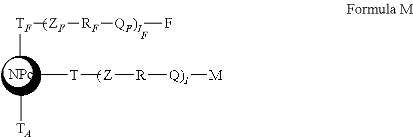

said step c taking place according to the following diagram

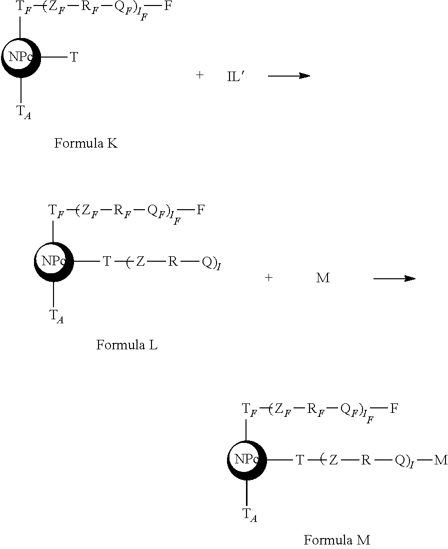

d. optionally a step of linking an addressing agent by optionally contacting said active nanoplatform of Formula M with an L_{A} linker precursor of Formula 3,

Z_{A}—R_{A}-Q_{A} Formula 3

to obtain an active nanoplatform of Formula N,

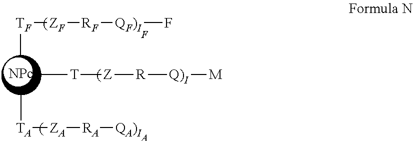

followed by bringing said active nanoplatform of Formula N into contact with said addressing agent A to obtain said active nanoplatform of Formula IX
said step d taking place according to the following diagram

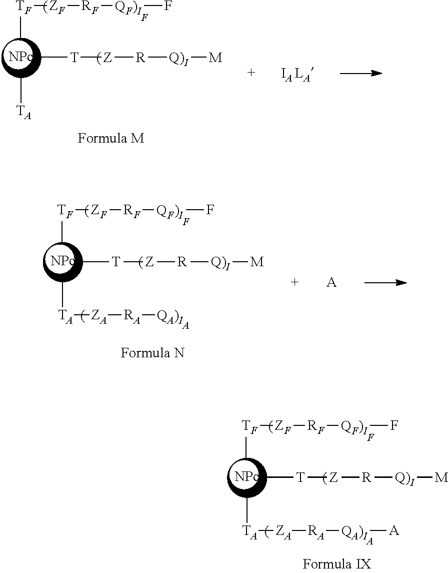

In this particular embodiment, the refunctionalization step is not total. OH and COOH grafting functions may remain on the surface of the nanoplatform.

In this embodiment, Formula b and Formula H are two different scripts of the same NPC₂ nanoplatform. The grafting functions T, T_{A} and T_{F} are chosen from the grafting functions available on the surface of the nanoplatform of Formula b, ie NH₂, OH and COOH.

In a particular embodiment, step a in the above process is:
a. a refunctionalization step by bringing an NPC₁ nanoplatform of Formula a-b into contact,

### Formula a-b

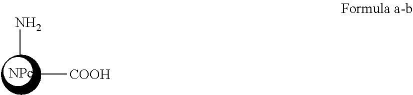

In which
NP_{C} has the meaning given in Formula I,
   with an α-ω diamino-alkane type organic molecule of 1 to 10 carbon atoms, comprising two primary amine functions, in particular 1,2-ethylenediamine, to obtain an NPC₂ nanoplatform of Formula bb or of Formula H,

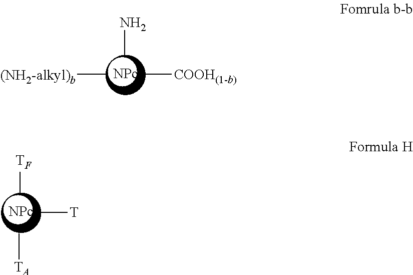

in which:
b is the rate of refunctionalization of the COOH grafting functions into NH2, b being a real number strictly greater than 0,
the T_{F}, T and T_{A} grafting functions are chosen from the grafting functions of the nanoplatform of Formula b-b, in particular NH₂ or Alkyl-NH₂,

In this particular embodiment, the refunctionalization step is not total. —OH and —COOH grafting functions may remain on the surface of the nanoplatform.

In this embodiment, Formulas b-b and Formula H are two different writings of the same NPC₂ nanoplatform. The grafting functions T, TA and TF are chosen from the grafting functions available on the surface of the nanoplatform of Formula b-b, ie NH₂, COOH.

In a particular embodiment, the process for the preparation as described above of said active nanoplatform of Formula X,

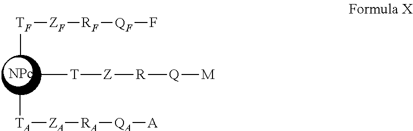

in which:
NP_{C}, T, T_{F}, T_{A}, M, F, A, Z, Z_{F}, Z_{A}, R, R_{F}, R_{A}, Q, Q_{F} and Q_{A} have the meanings of Formula I,
   comprises
a. a refunctionalization step by bringing into contact an NPC₁ nanoplatform of Formula a, Formula a

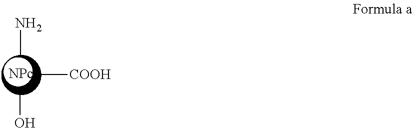

with an organic molecule of the α-ω diamino-alkane type comprising two amine functions, preferably two primary and/or secondary amine functions and even more preferably two primary amine functions, in order to obtain an NPC₂ nanoplatform of Formula b or of Formula H,

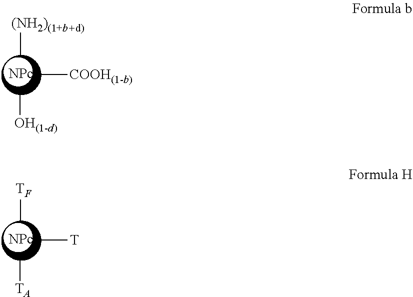

in which:
b, d, T_{F}, T_{A} and T having the meanings set out above, b. a step of binding a fluorophore by bringing a L_{F}′ linker precursor of Formula 1 into contact,

Z_{F}—R_{F}-Q_{F} Formula 1

with said NPC₂ nanoplatform of Formula H
to obtain a nanoplatform of Formula O

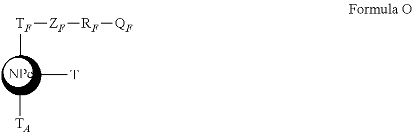

followed by bringing said nanoplatform of Formula O into contact with said fluorophore F to obtain a nanoplatform of Formula P,

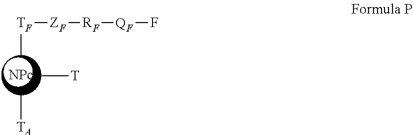

said step b taking place according to the following diagram

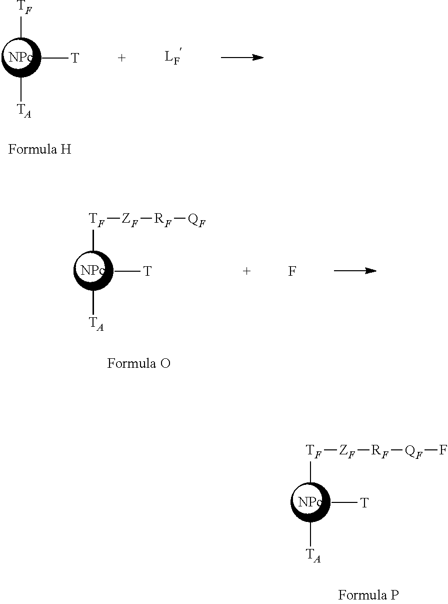

c. a step of grafting an active anti-tumor molecule by bringing said nanoplatform of Formula P into contact with a L′ linker precursor of Formula 2

Z—R-Q Formula 2

to obtain a nanoplatform of Formula Q,

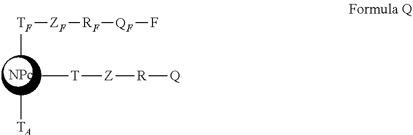

followed by bringing said nanoplatform of Formula Q into contact with said active antitumor molecule M to obtain an active nanoplatform of Formula R,

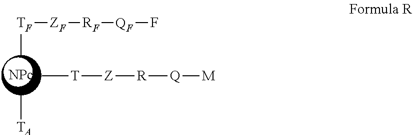

said step c taking place according to the following diagram

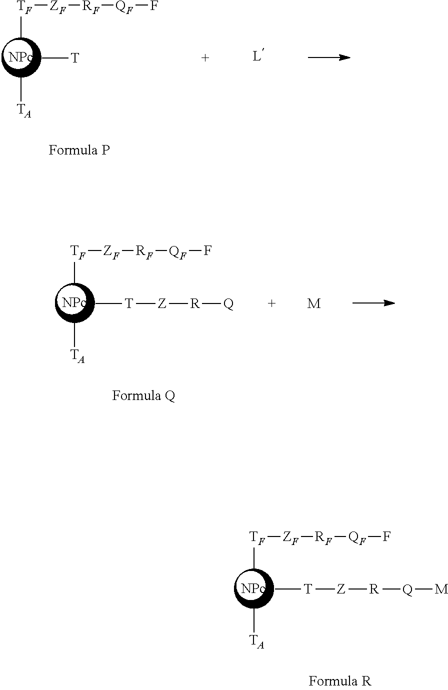

d. a step of linking an addressing agent by bringing said active nanoplatform of Formula R into contact with a linker precursor L′_{A} of Formula 3

Z_{A}—R_{A}-Q_{A} Formula 3

to obtain an active nanoplatform of Formula S,

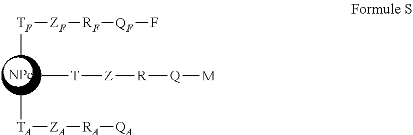

followed by contacting said active nanoplatform of Formula S with said addressing agent A to obtain said active nanoplatform of Formula X
said step d taking place according to the following diagram

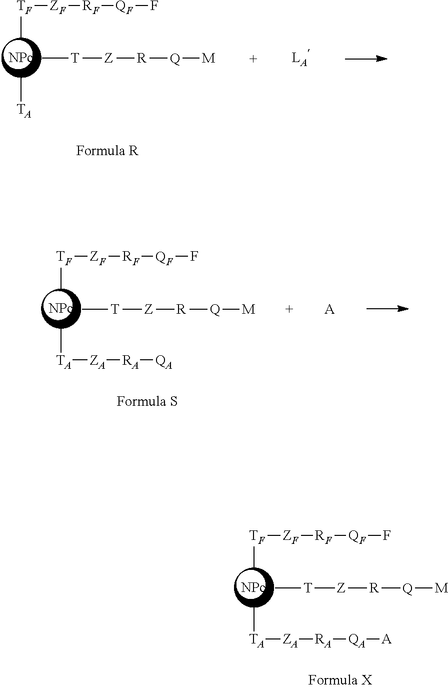

In this embodiment, the three molecules of interest are respectively bound by long linkers to the nanoplatform, the three linkers being able to be identical or different between them according to the needs for the active nanoplatforms

In a particular embodiment, step a in the above method is: a step of re-functionalization by bringing an NPC₁ nanoplatform of Formula a-b into contact,

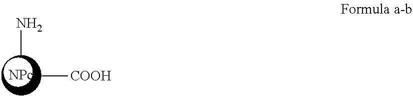

with an α-ω diamino-alkane type organic molecule of 1 to 10 carbon atoms, comprising two primary amine functions, in particular 1,2-ethylenediamine, to obtain an NPC₂ nanoplatform of Formula bb or of Formula H,

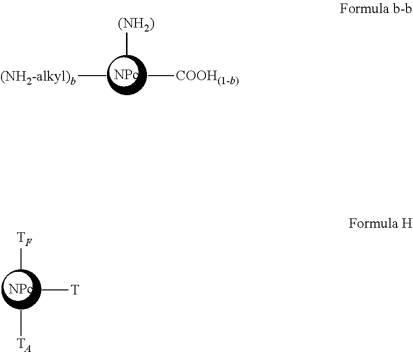

in which:
b, T_{F}, T_{A} and T have the meanings set out above,

In a particular embodiment, the method of preparation as described above of said active nanoplatform of Formula VI,

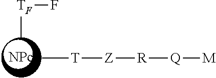

### Formula VI

in which:
NP_{C}, T, M, F, T_{F}, Z, R, and Q have the meanings of Formula I,
   comprises
a. a refunctionalization step by bringing into contact an NPC₁ nanoplatform of Formula a, Formula a

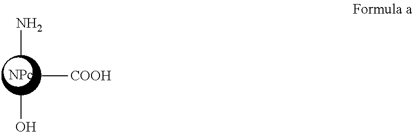

with an organic molecule of type α-ω diamino-alkane comprising two amine functions, preferably two primary and/or secondary amine functions and even more preferably two primary amine functions, in order to obtain an NPC₂ nanoplatform of Formula b or of Formula T,

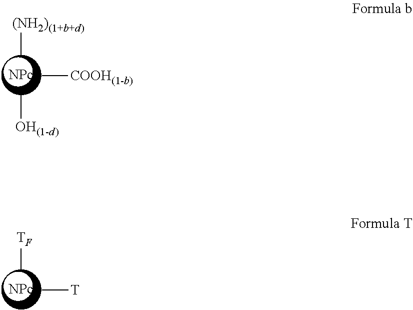

b, d, T and T_{F} having the meanings set out above,
b. a step of binding a fluorophore by bringing said fluorophore F into contact with an NPC₂ nanoplatform of Formula T,
   to obtain a nanoplatform of Formula U

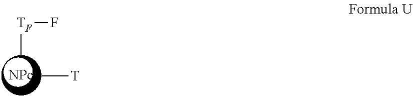

said step b taking place according to the following diagram

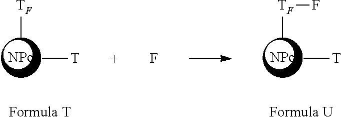

c. a step of grafting an active antitumor molecule by bringing said nanoplatform of Formula U into contact with a linker precursor L′ of Formula 2

Z—R-Q Formula 2

to obtain the nanoplatform of Formula W

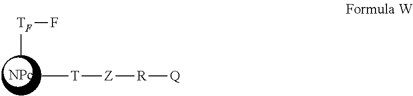

followed by bringing said nanoplatform of Formula W into contact with said active antitumor molecule M, to obtain said active nanoplatform of Formula VI,
said step c taking place according to the following diagram

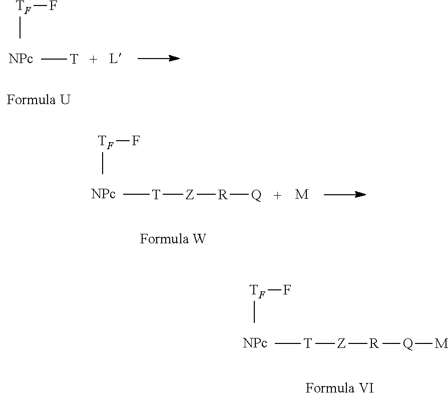

In this embodiment, the fluorophore is bound directly to the nanoplatform via a short linker, while the active antitumor molecule is bound to the nanoplatform via a long linker. In a particular embodiment, step a in the above process is: a step of re-functionalization by bringing into contact an NPC₁ nanoplatform of Formula a-b,

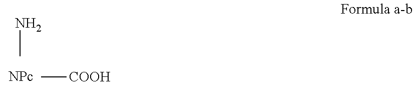

with an organic molecule of type α-ω diamino-alkane of 1 to 10 carbon atoms, comprising two primary amine functions, in particular 1,2-ethylenediamine, to obtain an NPC₂ nanoplatform of Formula b-b or of Formula T,

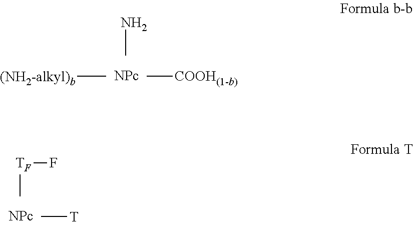

in which:
b, T and T_{F} have the meanings stated above,

In a particular embodiment, the method of preparation as described above of said active nanoplatform of Formula IV,

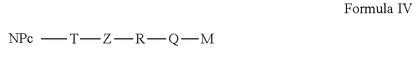

in which:
NP_{C}, T, M, Z, R and Q have the meanings of Formula I,
   comprises:
a. a refunctionalization step by bringing into contact an NPC₁ nanoplatform of Formula a,

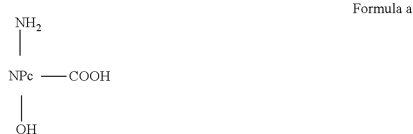

with an organic molecule of α-ω diamino-alkane type comprising two amine functions preferably two primary and/or secondary amine functions and even more preferably two functions primary amines, to obtain an NPC₂ nanoplatform of Formula b or Formula Y,

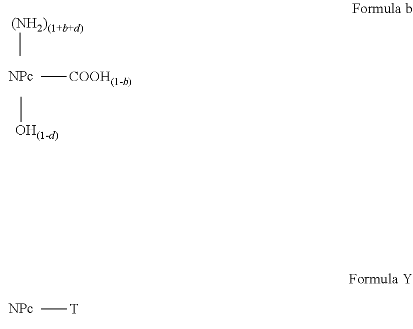

in which:
b, d and T having the meanings stated above,
b. a step of grafting an active anti-tumor molecule by bringing said nanoplatform of Formula Y into contact with a L′ linker precursor of Formula 2

Z—R-Q Formula 2

to obtain a nanoplatform of Formula Z

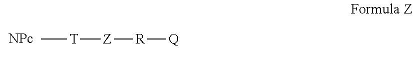

followed by bringing said nanoplatform of Formula Z into contact with said active antitumor molecule M, to obtain the active nanoplatform of Formula IV,
said step b taking place according to the following diagram

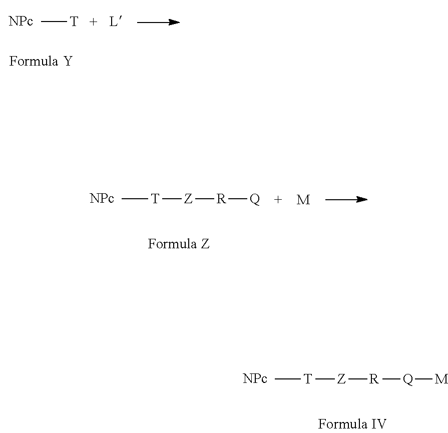

In a particular embodiment, step a in the above process is:
a refunctionalization step by bringing into contact an NPC₁ nanoplatform of Formula a-b,

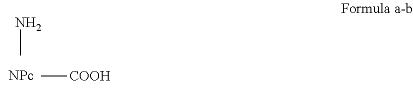

with an organic molecule of type α-ω diamino-alkane of 1 to 10 carbon atoms, comprising two primary amine functions, in particular 1,2-ethylenediamine, to obtain an NPC₂ nanoplatform of Formula b-b or of Formula Y,

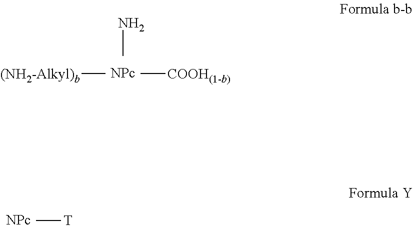

in which:
b, d and T have the meanings stated above,

In a particular embodiment, the process for the preparation as described above of said active nanoplatform of Formula VIII,

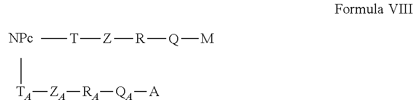

in which:
NP_{C}, T, T_{A}, M, A, Z, Z_{A}, R, R_{A}, Q, and Q_{A} have the meanings of the Formula I, comprises:
a. a refunctionalization step by bringing into contact an NPC₁ nanoplatform of Formula a,

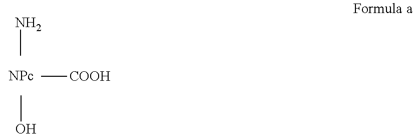

with an organic molecule of type α-ω diamino-alkane, comprising two amine functions preferably two primary and/or secondary amine functions and even more preferably two primary amine functions, in order to obtain an NPC₂ nanoplatform of Formula b or of Formula AA,

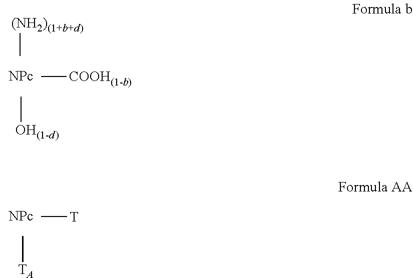

in which:
b, d, T and T_{A} having the meanings set out above,
b. a step of grafting an active anti-tumor molecule by bringing said nanoplatform of Formula AA into contact with a L′ linker precursor of Formula 2

Z—R-Q Formula 2

to obtain a nanoplatform of Formula AB

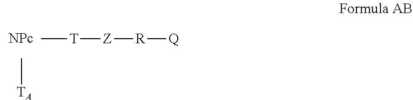

followed by bringing said nanoplatform of Formula AB into contact with said active antitumor molecule M, to obtain an active nanoplatform of Formula AC,

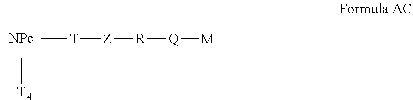

said step b taking place according to the following diagram

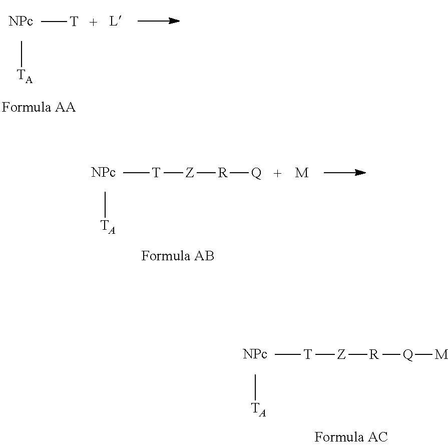

c. a step of linking an addressing agent by bringing said active nanoplatform of Formula AC into contact with a precursor of linker L′ₐ of Formula 3,

Z_{A}—R_{A}-Q_{A} Formula 3

to obtain an active nanoplatform of Formula AD

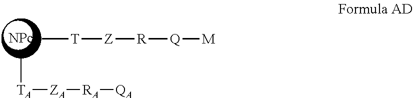

followed by bringing said active nanoplatform of Formula AD into contact with said addressing agent A to obtain said active nanoplatform of Formula VIII
said step c taking place according to the following diagram

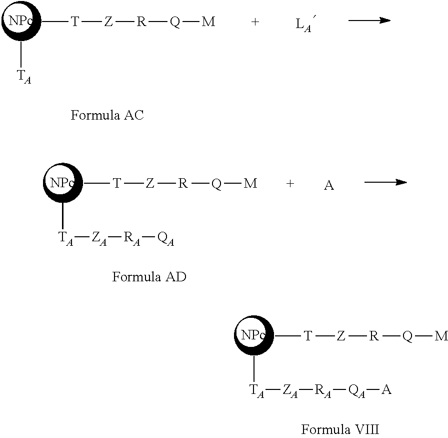

In a particular embodiment, step a in the above process is:
a refunctionalization step by bringing an NPC₁ nanoplatform of Formula a-b into contact,

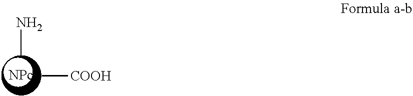

with an α-ω diamino-alkane organic molecule of 1 to 10 carbon atoms, having two primary amine functions, in particular 1,2-ethylenediamine, for obtain an NPC₂ nanoplatform of Formula bb or Formula AA,

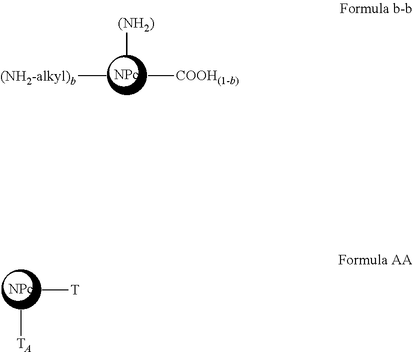

in which:
b, d, T and T_{A} have the meanings stated above,

In a particular embodiment, the preparation process as described above comprises, before the optional step of refunctionalizing an NPC₁ nanoplatform,

a step of synthesis of said NPC₁ nanoplatform of Formula a

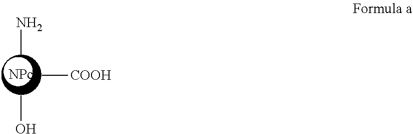

by bringing citric acid into contact with diethylenetriamine in water under a microwave for a sufficient time to obtain said nanoplatform of Formula a,
according to the scheme

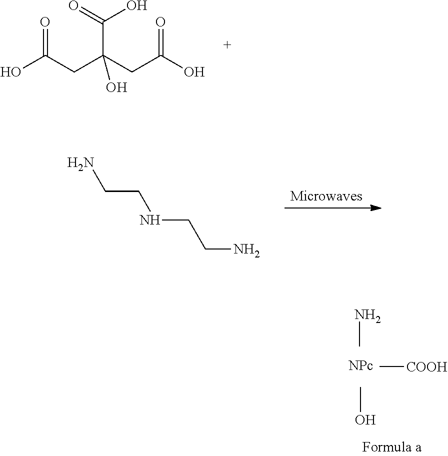

in which:
NP_{C} has the meaning of Formula I,

In this embodiment, the initial nanoplatform (NPC₁) is synthesized from organic molecules that can be biobased. This synthesis allows nanoplatforms to be obtained directly. If there is no refunctionalization step, the nanoplatforms obtained following this synthesis are those used to graft the molecules of interest.

In a particular embodiment, the preparation process as described above comprises, before the optional step of refunctionalizing an NPC₁ nanoplatform,

a step of synthesis of said NPC₁ nanoplatform of Formula a-b

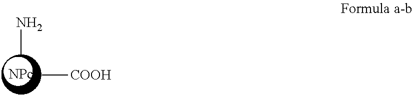

by bringing citric acid into contact with diethylenetriamine in water under microwaves for a time sufficient to obtain said nanoplatform of Formula a-b,
according to the scheme

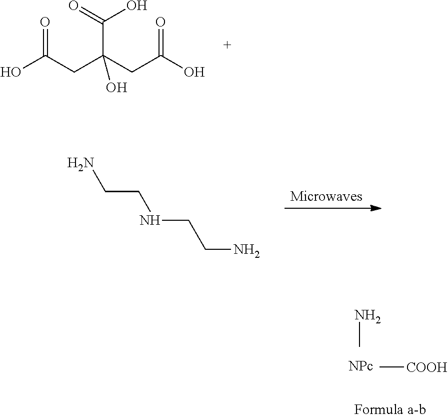

in which:
NP_{C} has the meaning of Formula I,

In this embodiment, the initial nanoplatform (NPC₁) is synthesized from organic molecules that can be biobased. This synthesis allows nanoplatforms to be obtained directly.

In a particular embodiment, the preparation process as described above comprises,

a step of refunctionalizing an NPC₁ nanoplatform of Formula a,

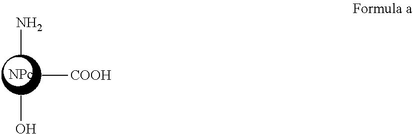

by bringing said nanopatform NPC₁ of Formula a into contact with an excess of 1,2-ethylenediamine at a temperature of 100 to 150° C. for 2 to 24 hours, in particular 12 hours, to obtain an NPC₂ nanoplatform of Formula b, according to the diagram,

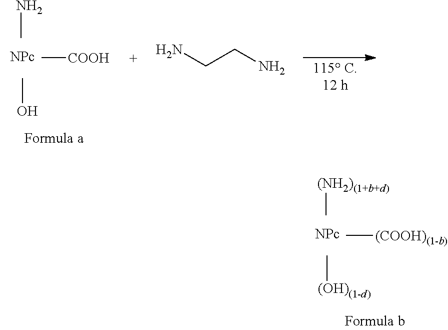

in which
b, d and NP_{C} have the meanings stated above,

In a particular embodiment, the preparation process as described above comprises, a step of refunctionalizing an NPC₁ nanoplatform of Formula a-b,

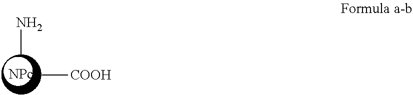

by bringing said NPC₁ nanopatform of Formula ab into contact with an excess of 1,2-ethylenediamine at a temperature of 100 to 180° C. for 2 to 24 hours, in particular 12 hours, to obtain an NPC₂ nanoplatform of Formula b-b,
according to the scheme,

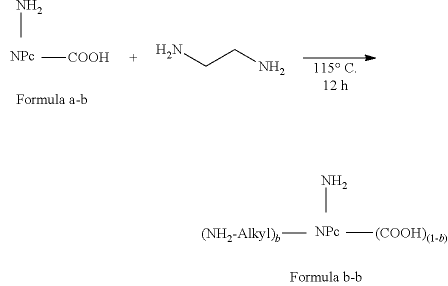

in which
b, d and NP_{C} have the meanings set out above,

In this embodiment, the alkyl group is: ethyl (—CH₂—CH₂—).

In this embodiment, the initial nanoplatform (NPC₁) after being synthesized is refunctionalized with an amino compound to increase the number of primary amine grafting functions on the surface of the nanoplatform. This step can be carried out with a primary diamine. This primary diamine may be 1,2-diaminoethane (1,2-ethylenediamine), 1,3-diaminopropane or 1,4 diaminobutane.

In this embodiment, the re-functionalization rate is 95%.

In a particular embodiment, the process for the preparation as described above of said active nanoplatform of Formula XII,

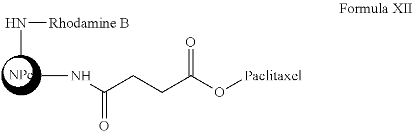

in which:
NP_{C} has the meaning of Formula I,
   comprises
a. a step of synthesis of an NPC₁ nanoplatform of Formula a,

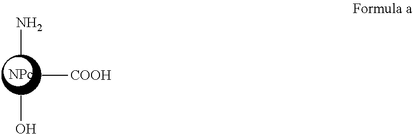

by bringing citric acid into contact with diethylenetriamine in water under microwaves with a power of 500 to 1000 W, in particular 600 W, for a time of 1 to 5 minutes, in particular 2 minutes, to obtain said nanoplatform of Formula a,
according to the scheme

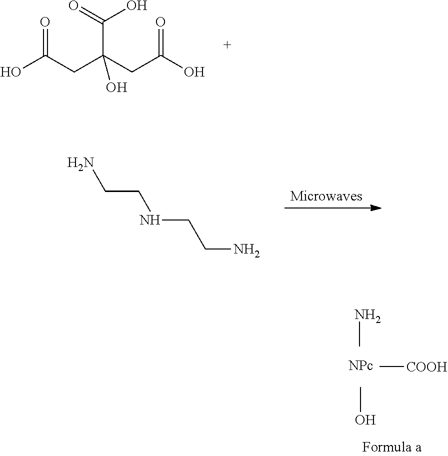

b. a step of refunctionalizing said NPC₁ nanoplatform of Formula a, by bringing said NPC₁ nanoplatform of Formula a into contact with an excess of 1,2-ethylenediamine at a temperature of 100 to 150° C. for 2 to 24 hours, in particular 12 hours, to obtain an NPC₂ nanoplatform of Formula b or Formula AE,

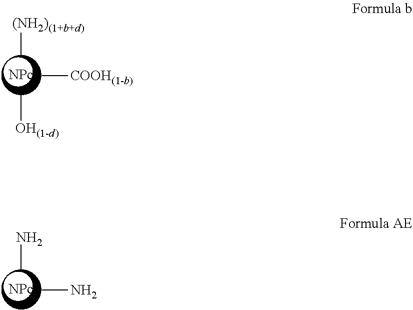

in which:
b and d have the meaning of Formula I,
   according to the scheme,

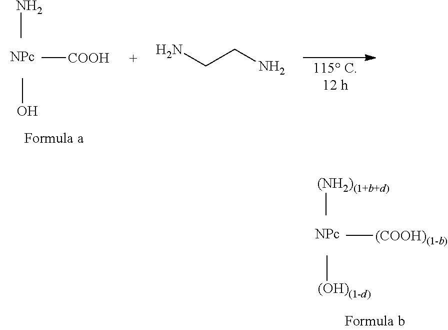

c. a step of linking a fluorophore by bringing a fluorophore F constituted by Rhodamine B into contact with said nanoplatform NPC₂ of Formula AE,
   to obtain a nanoplatform of Formula AF

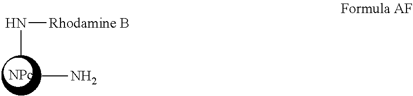

said step c taking place according to the following diagram

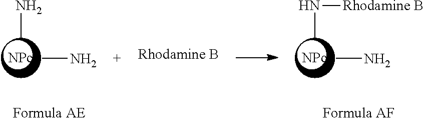

d. a step of grafting an active antitumor molecule by bringing said nanoplatform of Formula AF into contact with a linker precursor L′, succinic anhydride and a base, Na₂CO₃, to obtain the nanoplatform of Formula AG

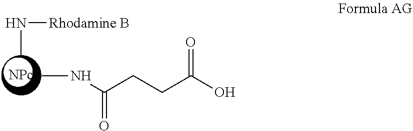

followed by bringing said nanoplatform of Formula AG into contact with said active antitumor molecule M, paclitaxel, to obtain said active nanoplatform of Formula XII,
said step d taking place according to the following diagram

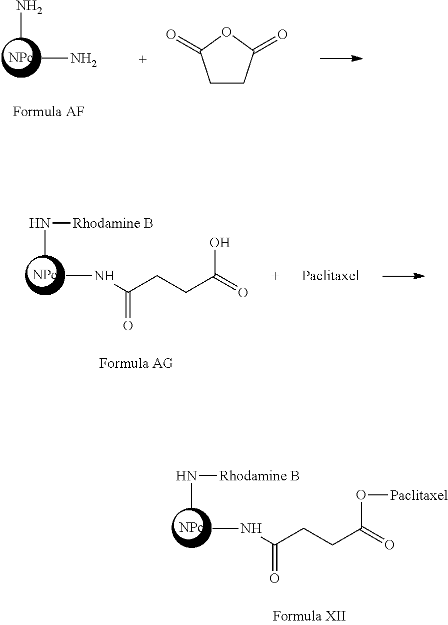

In a particular embodiment, the process for the preparation as described above of said active nanoplatform of Formula XII

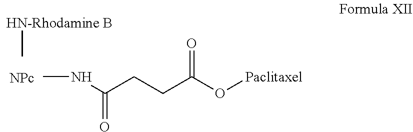

in which:
NP_{C} has the meaning of Formula I,
   comprises
a. a step of synthesis of an NPC₁ nanoplatform of Formula a-b,

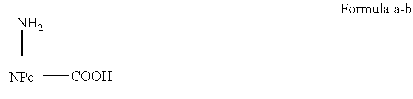

by bringing citric acid into contact with diethylenetriamine in water under microwaves with a power of 500 to 1000 W, in particular 600 W, for a time of 1 to 5 minutes, in particular 2 minutes, to obtain said nanoplatform of Formula a-b,
according to the scheme

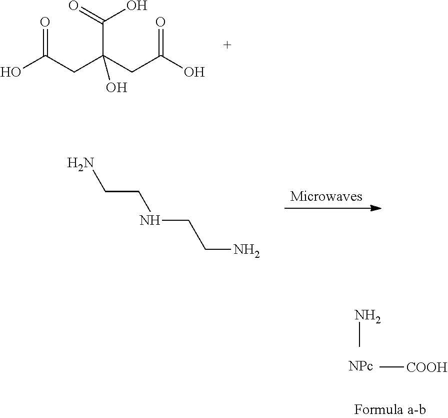

b. a step of refunctionalizing said NPC₁ nanoplatform of Formula ab, by bringing said NPC₁ nanoplatform of Formula ab into contact with an excess of 1,2-ethylenediamine at a temperature of 100 to 180° C. for 2 to 24 hours, in particular 12 hours, to obtain a NPC₂ nanoplatform of Formula bb or Formula AE,

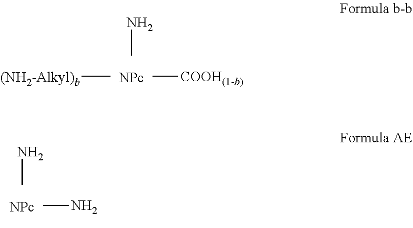

b has the meaning given above
   according to the scheme,

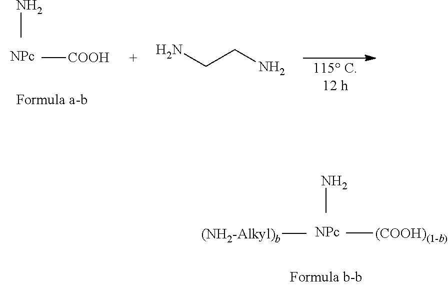

c. a step of linking a fluorophore by bringing a fluorophore F constituted by Rhodamine B into contact with said NPC₂ nanoplatform of Formula AE,
   to obtain a nanoplatform of Formula AF

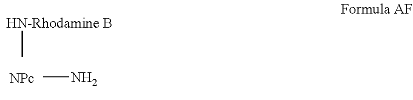

said step c taking place according to the following scheme

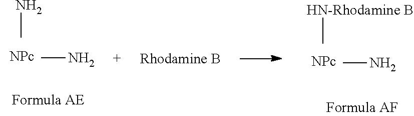

d. a step of grafting an active antitumor molecule by bringing said nanoplatform of Formula AF into contact with a linker precursor L′, succinic anhydride, and a base, in particular sodium carbonate or diisopropylethylamine, to obtain the nanoplatform of Formula AG

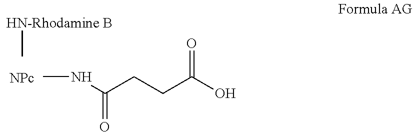

followed by bringing said nanoplatform of Formula AG into contact with said active antitumor molecule M, paclitaxel, in order to obtain said active nanoplatform of Formula XII,
said step d taking place according to the following diagram

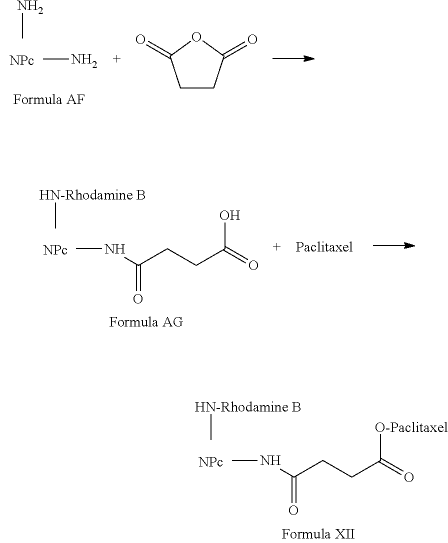

According to another aspect, the invention relates to an active nanoplatform in which the NPC₂ nanoplatform
does not exhibit cellular toxicity in vitro; and
is likely to be internalized by cells in culture in vitro,
   at a concentration of 1 μg/ml to 25 μg/ml.

According to a more particular aspect, the invention relates to an active nanoplatform in which the NPC₂ nanoplatform
does not exhibit cell toxicity in vitro, in particular on the cancer cell line U-87-MG and on the non-cancer lines FIMEC-1 and NHDF; and
is likely to be internalized by cells in culture in vitro, in particular by the cancer cell line U-87-MG,
   at a concentration ranging from 1 μg/ml to 25 μg/ml.

The present invention also relates to the use of the active nanoplatform previously described as a medicament.

The present invention also relates to the active nanoplatform previously described for its use as a medicament.

As such, the invention relates to a pharmaceutical composition comprising at least one active nanoplatform described above in combination with a pharmacologically acceptable excipient.

According to a particular aspect, the subject of the invention is a pharmaceutical composition comprising at least one active nanoplatform described above where said active antitumor molecule M is chosen from taxanes or anthracyclines, and in combination with a pharmacologically acceptable excipient.

According to a more particular aspect, the invention relates to a pharmaceutical composition comprising at least one active nanoplatform described above where said active antitumor molecule M is paclitaxel or doxorubicin, and in combination with a pharmacologically acceptable excipient.

According to a particular aspect, the invention relates to a pharmaceutical composition comprising at least one active nanoplatform described above where said active antitumor molecule M is paclitaxel, and in combination with a pharmacologically acceptable excipient.

According to an even more particular aspect, the subject of the invention is a pharmaceutical composition comprising at least one active nanoplatform as described above, in which said active anti-tumor molecule M is chosen from taxanes, more particularly paclitaxel and docetaxel or from anthracyclines, more particularly epirubicin, pirarubicin, idarubicin, zorubicin, aclarubicin and doxorubicin and said active nanoplatform being in association with a pharmacologically acceptable excipient.

The pharmaceutical compositions according to the invention advantageously comprise one or more excipients or vehicles, acceptable from the pharmaceutical point of view. Mention may be made, for example, of saline, physiological, isotonic, buffered solutions, etc., compatible with pharmaceutical use and known to those skilled in the art. The compositions may contain one or more agents or vehicles chosen from dispersants, solubilizers, stabilizers, preservatives, etc. Agents or vehicles which can be used in formulations (liquids and/or injectables and/or solids) are in particular methylcellulose, hydroxy methylcellulose, carboxymethylcellulose, polysorbate 80, mannitol, gelatin, lactose, acacia, etc. The compositions can be formulated in the form of solutions or injectable suspensions, gels, tablets, powders, capsules, capsules, etc.

Furthermore, the invention, which contains for example 45 mg of paclitaxel per g of NPC₂ (NMR data), also comprises a pharmaceutical composition formulated in unit dose comprising from 1 g to 15 g of active nanoplatforms, in particular from 3 g to 12, 5 g of active nanoplatforms and preferably from 5 g to 10 g of active nanoplatforms. In other words, the pharmaceutical composition according to the invention is formulated in unit dose of 1; 1.5; 2; 2.5; 3; 3.5; 4; 4.5; 5; 5.5; 6; 6.5; 7; 7.5; 8; 8.5; 9; 9.5; 10; 10.5; 11; 11.5; 12; 12.5; 13; 13.5; 14; 14.5 and/or 15 g of active nanoplatforms.

According to another aspect, the invention relates to a pharmaceutical composition formulated at a dose of active nanoplatforms from 20 mg/kg to 200 mg/kg of body weight (or from 0.88 g/m² to 8.8 g/m² of body surface area).

According to another particular aspect, the subject of the invention is a pharmaceutical composition formulated at a dose of active nanoplatforms from 20 mg/kg to 200 mg/kg of body weight, in particular from 40 mg/kg to 180 mg/kg, particularly 60 mg/kg to 160 mg/kg and preferably from 80 mg/kg to 140 mg/kg. In other words, the pharmaceutical composition according to the invention is formulated in a unit dose of 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195 and/or 200 mg/kg of active nanoplatforms.

According to a particular aspect, the invention relates to a pharmaceutical composition formulated at a dose of active nanoplatforms of 0.88 g/m² to 8.8 g/m² of body surface, in particular of 1 g/m² to 8 g/m², particularly from 3 g/m² to 6 g/m² and preferably from 3 g/m² to 6 g/m². In other words, the pharmaceutical composition according to the invention is formulated in a unit dose of 0.88; 1; 1.5; 2; 2.5; 3; 3.5; 4; 4.5; 5; 5.5; 6; 6.5; 7; 7.5; 8; 8.5 and/or 8.8 g/m² of active nanoplatforms.

In order to be administered to a mammal, in particular to humans, the subject of the invention is a pharmaceutical composition formulated to be administrable by any suitable route of administration, in particular by enteral or parenteral route.

Among the enteral routes, mention may in particular be made of the buccal, sublingual, perlingual or rectal route. Among the parenteral routes, there may be mentioned in particular the intravenous injection, the subcutaneous injection, the intradermal injection, the intramuscular injection, the intraperitoneal injection, the intrathecal injection or the intratumoral injection. The vaginal, nasal, pulmonary, auricular, ophthalmic or transdermal route of administration is also possible. The injections can be given as a bolus, and/or by continuous infusion.

According to a preferred mode of administration, the pharmaceutical composition according to the invention is formulated to be administered by the enteral route chosen from the buccal, sublingual, perlingual or rectal route. Preparations for parenteral administration may include sterile aqueous or non-aqueous solutions, suspensions or emulsions. Examples of non-aqueous solvents are propylene glycol, polyethylene glycol, vegetable oils, such as olive oil, or injectable organic esters such as ethyloleate. Aqueous vehicles include water, alcohol/water solutions, emulsions or suspensions.

According to another preferred mode of administration, the pharmaceutical composition according to the invention is formulated to be administered by parenteral route chosen from intravenous, subcutaneous, intradermal, intramuscular, intraperitoneal, intrathecal or intratumoral injection.

According to another preferred mode of administration, the pharmaceutical composition according to the invention is formulated to be administrable by the subcutaneous route.

Another aspect of the invention relates to active nanoplatforms for their use in the treatment of cancers, in particular primary brain tumors and more particularly glioblastoma and/or brain metastases originating from primary tumors of non-cerebral localization.

A more specific aspect of the invention relates to active nanoplatforms for their use in the treatment of glioblastoma and brain metastases originating from different primary tumors of non-cerebral localization.

An even more specific aspect of the invention relates to active nanoplatforms for their use in the treatment of glioblastoma.

An even more precise aspect of the invention relates to active nanoplatforms for their use in the treatment of brain metastases originating from different primary tumors of non-cerebral localization.

A particular aspect of the invention comprises active nanoplatforms in which the bond between paclitaxel or doxorubicin and the linker L of the nanoplatform of Formula 20 is cleavable and allows the release of paclitaxel or doxorubicin, in particular by breaking up by hydrolysis the ester bond between the linker L and paclitaxel or doxorubicin,

A very specific aspect of the invention comprises active nanoplatforms in which the bond between paclitaxel and the linker L of the nanoplatform of Formula 20 is cleavable and allows the release of paclitaxel, in particular by breaking the ester bond between the linker by hydrolysis L and paclitaxel.

### BRIEF DESCRIPTION OF THE DRAWING FIGURES

The file of this patent contains at least one drawing executed in color. Copies of this patent with color drawings will be provided by the Patent and Trademark Office upon request and payment of the necessary fee.

Another very precise aspect of the invention comprises active nanoplatforms in which the bond between the doxorubicin and the linker L of the nanoplatform of Formula 20 is cleavable and allows the release of the doxorubicin, in particular by breaking the ester bond by hydrolysis the L linker and doxorubicin. Figures:

FIG. 1A represents an image of the NPC₁ or NPC₂ nanoplatforms obtained by transmission electron microscopy (TEM). The scale is 50 nm, and the visual distribution of the size of the nanoplatforms is significantly lower than this scale.

FIG. 1B represents, the diagram distribution of the size of the nanoplatforms obtained, and the curve modeling this distribution by a log-normal function. The distribution is centered around a size of 17 nm.

FIG. 2 represents the diffractogram of the NPC₁ or NPC₂ nanoplatforms obtained by DRX (measurements carried out on powder using a Bruker D2 Phaser X-ray powder diffractometer). The curve is representative of an amorphous compound.

FIG. 3A represents the Raman spectrum of the NPC₁ or NPC₂ nanoplatforms (measurements made on powder using a Raman Explora microscope equipped with an air objective (50×) and operating at 785 nm). The presence of an intense band at 1575 cm⁻¹ (G band of graphite), the absence of a band at 2714 cm⁻¹ (2D band of graphite), and the weak band between 1310 and 1350 cm⁻¹ (Band D of graphite) do not suggest the presence of graphite in the sample.

FIG. 3B represents the infrared spectrum of the NPC₁ or NPC₂ nanoplatforms (measurements carried out on a KBr pellet using a PerkinElmer Spectrum 100 Optica spectrometer).

FIG. 4A represents the XPS spectrum of the NPC₁ or NPC₂ nanoplatforms (measurements performed on powder using a K-alpha spectrometer) for carbon.

FIG. 4B represents the XPS spectrum of NPC₁ or NPC₂ nanoplatforms (measurements performed on powder using a K-alpha spectrometer) for nitrogen. The peak is centered around a binding energy of 399.8 eV.

FIG. 4C represents the XPS spectrum of the NPC₁ or NPC₂ nanoplatforms (measurements performed on powder using a K-alpha spectrometer) for oxygen. The first peak is centered around a binding energy of 531.0 eV, the second 532.2 eV and the third 533.2 eV.

FIG. 5A shows the emission (dotted curve) and absorption (solid curve) spectra of nanoplatforms (measurements carried out in solution in water using a Fluoro-Max fluorimeter and a Jasco V-570 UV-Visible spectrometer respectively) NPC₁ or NPC₂.

FIG. 5B represents the intensity of the fluorescence emission of the NPC₁ or NPC₂ nanoplatforms as a function of the incident wavelength and of the emitted wavelength. The optimal corresponds to an incident wavelength of 360 nm for an emission of 455 nm.

FIG. 6 represents the emission spectrum of the NPC₁ or NPC₂ nanoplatforms, at 37° C. and with a concentration of 25 μg/ml in a biological medium (culture medium+fetal calf serum), as a function of time, and with an incident wavelength at 360 nm.

FIG. 7 represents the colorimetric tests measuring the survival of the cells, carried out after 72 h of incubation of NPC₂ (A, B and C). The real-time impedance test measuring cell survival is carried out for at least 72 h of incubation of NPC₂ (D, E and F). These tests were carried out with doses of 1 to 100 μg/ml on U-87-MG cells (A and D), HMEC-1 (B and E) and on NHDF (C and F).

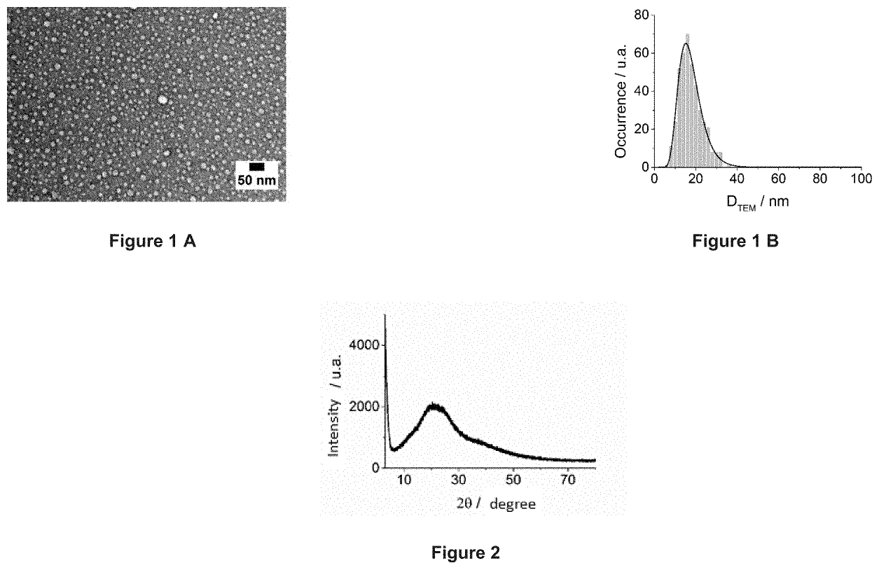

### DESCRIPTION OF THE PREFERRED EMBODIMENTS

Panel A, B and C: abscissa axis=Concentration in g/ml and the ordinate axis=cell viability in %. For this whole panel,
the MTT test is represented by dashes marked with a diamond,
the Sulforhodamine B test is represented by dotted lines marked with a square, and
the Alamar Blue test is represented by a line marked with a triangle.

Panel D, E and F: abscissa axis=time in h and ordinate axis=cell viability in %. For this whole panel,
the dose of 5 μg/ml is represented by a dark gray line
the dose of 25 μg/ml is represented by a very light gray line
the dose of 50 μg/ml is represented by a light gray line
the dose of 100 μg/ml is represented by a deep black line.

FIG. 8 represents the colorimetric tests measuring the survival of the cells, carried out after 72 h of incubation of NPC₂. These tests were carried out with doses of 1 to 100 μg/ml on HMEC-1, U-87 MG, GL261 DsRed, OLN-93, C8-D1A cells.

abscissa axis=Concentration in μg/ml and ordinate axis=cell viability in %.

FIG. 9 shows the internalization of NPC₂ in U-87-MG, HMEC-1, and NHDF cells after a 4 h incubation (marked NPC₂ 1 μg/ml and NPC₂ 5 μg/ml) with orthogonal views showing the distribution of NPC₂ in the thickness of the cells. The internalization of NPC₂ by HMEC-1 and NHDF is negligible while IT internalization in U-87-MG cells is visible. This observation is made from two-photon fluorescence images obtained at an excitation of 740 nm and emissions from 480 to 550 nm after correction of the autofluorescence, as well as images obtained in transmitted light to locate the cells.

### Scale bar: 35 μm

FIG. 10 shows the internalization of NPC₂ in U-87-MG, HMEC-1, and NHDF cells after an incubation of 4 h and an NPC₂ concentration of 25 μg/ml. U-87-MG cells and NHDF cells do not show any major change in the internalization of NPC₂ compared to FIG. 8. In contrast, the internalization of NPC₂ at 25 μg/ml is improved in HMEC-1 cells as observed from two-photon fluorescence images after autofluorescence correction. Transmitted light images are also provided to locate cells.

### Scale bar: 35 pm

FIG. 11 shows the internalization of NPC₂ in U-251 MG cells after an incubation of 4 h and an NPC₂ concentration of 1, 5 and/or 25 μg/ml. Control cells were exposed to the culture medium only. An internalization of the NPC₂ in these cells is demonstrated, as shown by the two-photon fluorescence images obtained at an excitation of 740 nm and of the emissions from 480 to 550 nm after correction of the autofluorescence. Transmitted light images are also provided to locate cells.

### Scale bar: 35 pm

FIG. 12 represents the internalization of NPC₂ in U-87 MG cells with a concentration of NPC₂ nanoplatforms of 0 (controls), 1 or 5 μg/ml under conditions of blocking or not endocytosis (without blocking, inhibition energy-dependent endocytosis, clathrin inhibitor, caveolin inhibitor, lipid raft inhibitor).

NPC₂ are internalized in U-87-MG cells after treatment with concentrations of 1 and 5 μg/ml for 4 hours in the absence of inhibition of endocytosis (positive control). When these same cells are cultured at 4° C. in order to block energy-dependent endocytosis or to endocytosis inhibitors using the caveolin pathway or lipid rafts, we do not find NPC₂ inside the cells which testifies to the implication of these endocytosis pathways in the cellular internalization of NPC₂. On the other hand, those in the presence of an endocytosis inhibitor using the clathrin pathway remain capable of internalizing the NPC₂ at concentrations of 1 and 5 μg/ml. Transmitted light images are provided to locate cells. All two-photon fluorescence images obtained at an excitation of 740 nm with an emission of 480 to 550 nm are corrected by subtracting the autofluorescence from the untreated control cells.

### Scale bar: 35 pm

FIG. 13 represents the comparative cell viability tests between paclitaxel alone (PTX) and the active nanoplatforms of Example 3.1 (NPC₂-PTX) on HMEC-1 cells (A); SK-N-SH (B) and U-87 MG (C), and measured by the MTT test, after 72 hours of treatment with a concentration range from 0.1 to 100 nM expressed in PTX or PTX equivalent carried by NPC₂, or measured by the impedance test in real time after 72 h of treatment of HMEC-1 (D) for the active nanoplatforms of Example 3.1. These data made it possible to determine the IC₅₀s of paclitaxel and of the active nanoplatforms of Example 3.1 according to the line considered.

X axis=Concentration of PTX or PTX equivalent carried by NPC₂ in nM and the y axis=cell viability in %

FIG. 14 represents the MTT tests measuring cell survival, performed after 72 h of incubation of NPC₂-PTX. These tests were carried out with doses of 0.1 to 10 μM on the U-87-MG and GL261 cells.

X axis=Concentration in mM and y axis=cell viability in %.

FIG. 15 represents the pharmacological activity of PTX (A and C) and of NPC₂-PTX (B and D), analyzed by indirect immunofluorescence of tubulin on HMEC-1 cells (A and B) and U-87 MG (C and D) after 6 h of treatment with concentrations of PTX or equivalent PTX carried by NPC₂ of 10, 50 and/or 100 nM.

Scale bar: 0.5 cm=10 pm

FIG. 16 represents the pharmacological activity of NPC₂-PTX and NPC₂, analyzed by indirect immunofluorescence of tubulin on U-87 MG cells after 24 h of treatment with NPC₂-PTX concentrations of equivalent PTX carried by NPC₂ of 5 and 101 μM and NPC₂ concentrations of 38 and 76 μg/ml.

Scale bar: 0.5 cm=10 pm

FIG. 17 represents the pharmacological activity of PTX and of NPC₂-PTX, determined by optical microscopy on U-87 MG cells in 3D culture before treatment (D0) and 7 and 14 days after treatment with concentrations which correspond to the IC₅₀ calculated in 2D (equitoxic doses), 2.8 times the IC₅₀ and 4 times the IC₅₀.

### Scale bar: 500 pm

FIG. 18 represents the activity of PTX and NPC₂-PTX, measured on U-87 MG cells in 3D culture by the Alamar Blue test 7 days (A) and 14 days after treatment (B). The results are expressed as a percentage relative to the control standardized to 100%.

X-axis=Concentration in multiple of the IC₅₀ and the y-axis=cell viability in %
X-axis=Concentration in multiple of IC₅₀ and y-axis=cell viability in %

FIG. 19 represents the pharmacological activity of NPC₂ and NPC₂-PTX, determined by optical microscopy on GL261 spheroid cells in 3D culture before treatment (D0) and 7 and 14 days after treatment with an NPC₂-PTX concentration of 20 μM and an NPC₂ concentration of 153 μg/ml.

FIG. 20 represents the pharmacological activity of NPC₂ and NPC₂-PTX, determined by optical microscopy on GL261 spheroid cells in 3D culture before treatment (D0) up to 14 days after treatment with NPC₂-PTX concentrations of 10 and 20 μM and NPC₂ concentrations of 76 and 153 μg/ml

X-axis=time after treatment in days=normalized cell surface

FIG. 21 represents the pharmacological activity of NPC₂ and NPC₂-PTX, determined by light microscopy on GL261 spheroid cells in 3D culture 14 days after treatment with an NPC₂-PTX concentration of 20 μM and an NPC2 concentration of 153 μg/ml

X-axis=Concentration of nanoparticles and y-axis=cell viability in %

FIG. 22 shows the monitoring of body weight in C57BL/6 mice up to 14 days after treatment with a dose of NPC₂-PTX of 11 mg/kg and a dose of NPC₂ of 2 mg/kg

X-axis=time after treatment in days and y-axis=average weight per group (g)

### EXAMPLES

### Example 1: Synthesis of the Initial NPC₁Nanoplatform and the NPC₂ Nanoplatform

### 1. Synthesis of the Initial NPC₁ Nanoplatform

In a 50 ml beaker, citric acid monohydrate (5.25 g, 25.0 mmol) is dissolved in distilled water (5 ml), then diethylene triamine is added dropwise (3.0 ml, 2.87 g, 27.6 mmol). The resulting yellow aqueous solution is heated using a microwave oven for 2 minutes at 600 W of power. After the residue has cooled, ethanol (25 ml) is added and the residue is scraped off with a spatula until a slightly brown powder is formed. After sonication with ultrasound for 2 minutes, the homogeneous suspension is centrifuged at 6000 rpm for 10 minutes. The brownish powder at the bottom of the centrifugation cylinder is collected and washed with ethanol and then with diethyl ether giving 4.8 g of initial NPC₁ nanoplatforms in brown powder after complete drying under vacuum, i.e. a mass yield of 60%.

### 2. Synthesis of the NPC₂ Nanoplatform

NPC₁ nanoplatforms (200 mg) are dissolved in 1 ml of ethylenediamine.

This solution is kept at a temperature of 115° C. with stirring for 12 hours. The brown solution obtained is cooled to room temperature and then added dropwise to 20 ml of dichloromethane with stirring. A suspension is then obtained. This is centrifuged at 6000 rpm for 10 minutes. The pellet is resuspended in 20 ml of dichloromethane using ultrasound for 5 minutes and then centrifuged again under the same conditions as above. This process is repeated a third time. The brown powder obtained is then dried under vacuum (0.1 torr) at 50° C. for 2 h. 190 mg of dry powder are obtained, ie a mass yield of 95% relative to NPC₂. The Kaiser test allows the determination of the number of primary amine functions on the surface of the nanoparticles, i.e. 2.7 m_{equivalents} of NH₂/g. The same reaction carried out with 1,3 diaminopropane at 135° C. for 12 h leads to 185 mg of dry powder (mass yield=92.5%/NPC₂) carrying 3 m_{equivalents} of NH₂/g of nanoparticles.

### Example 2: Characteristic of the NPC₂ Nanoplatform

### 1. Composition of the NPC₂ Nanoplatform

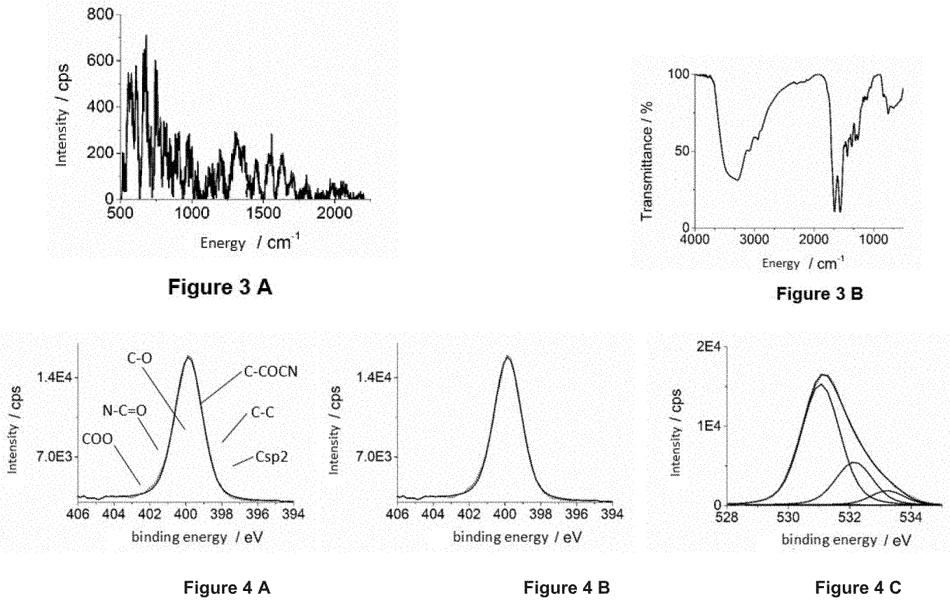

Due to the size of the nanoplatform obtained, the use of the XPS technique makes it possible to know the internal and surface bonds of the nanoplatform and therefore the atoms making up the nanoplatform. The NPC₂ nanoplatforms in powder form are mechanically “anchored” in a matrix in ultra-pure indium (by pressure), then the whole is analyzed by a K-alpha spectrometer. The results, presented in FIG. 3, show the presence of the following bonds in the nanoplatforms: C═C, C—C, C—CO, C—CN, C—O, C—N, N—C=0, C—NHR and COO.

### 2. Measurement of the Solubility of the NPC₂ Nanoplatform

Due to the very high solubility of nanoplatforms in water, combined with very high absorptivity, an absorption technique is impossible to use. Solubility was measured by visual inspection only. For this measurement, nanoplatform powder as obtained above is added to 64 mg of water. The solution is stirred for a few minutes. With an addition of 16.7 mg of nanoplatform, the solution after shaking is strongly colored and clear to the eye. The solubility of nanoplatforms is therefore greater than 250 g/l.

### 3. Hardness of the NPC₂ Nanoplatform

The hardness of the nanoplatforms was measured by atomic force microscopy (AFM). The atomic force microscope used is a Bruker Dimension Icon model and the tip used is a ScanAsyst-Air tip with a radius of curvature of 2 nm. Young's module is measured using the device's peak force mode. An aqueous solution of nanoplatforms is deposited on a glass microscope slide, then the Young's modulus of the sample is measured and is compared with two references: a polystyrene film which has a Young's modulus of 3-3.5 GPa and a PDMS film which has a Young's module of 3.5 MPa. Several nanoplatform deposits have been made. The Young's modulus values measured for the various samples are between 1 and 4 GPa. We can therefore conclude that nanoplatforms have a hardness close to that of polystyrene (organic polymer).

4. Density of the NPC₂ nanoplatform

The density of the nanoplatform is measured on an AccuPyc II 1340 pycnometer from nanoplatforms in powder form and using the helium gas expansion technique. Ten successive measurements are made, then the density value is calculated as the average of these ten measurements, giving a density of 1.53.

### 5. Dry Size of the NPC₂ Nanoplatform

The measurement of the “dry” size of the nanoplatforms is made on a transmission electron microscope (HITACHI H7650 at 80 kV). A drop of aqueous nanoplatform solution is placed on a copper grid (covered with a carbon film) previously positively charged using the “Glow discharge” technique (in order to increase the affinity of nanoplatforms for the grid). After one minute, the excess aqueous solution is removed by capillary action using absorbent paper and then a drop of an aqueous solution of uranyl acetate is added to the grid for one minute. After this time, the excess aqueous solution of uranyl acetate is removed by capillary action using absorbent paper. The operation is repeated twice for the contrasting agent (uranyl acetate), then the grid, once dry, is ready for observation. The size of the nanoplatforms is measured randomly on several shots using the ImageJ software (number of particles measured>400).

The average size obtained by TEM of the nanoplatforms is 17 nm.

### 6. Crystallinity of the NPC₂ Nanoplatform

a. By DRX

Nanoplatforms in powder form are analyzed on a Bruker D2 Phaser X-ray powder diffractometer. For information, the graphite has a very fine and intense line corresponding to the plane (002) at small angles (2θ˜26°), and as shown by the X-ray powder diffraction spectrum of the nanoplatforms in FIG. 2, it there is no trace of this characteristic graphite line.

b. By Raman Spectroscopy

Nanoplatforms in powder form are dispersed on a microscope slide, then the Raman spectra are measured on this slide using the Explan Raman microscope using an air objective (50×) and a laser at 785 nm.

Graphite is characterized by several bands in Raman spectroscopy, the G band which is located at 1575 cm⁻¹, the D band which is located between 1310 and 1350 cm⁻¹, and the 2D band which is located at 2714 cm⁻¹ (for a laser at 785 nm)

In fact, on the Raman spectrum of nanoplatforms (FIG. 3), we can observe a band at 1583 cm which could be the G band, however, there is no certainty on the presence of the D band. Finally, the 2D band is absent from the spectrum. It should however be noted that in certain cases, described in the literature, the 2D band may be absent for carbon nanoplatforms.

Measurement by DRX and by Raman suggests the substantially amorphous nature of the nanoplatform.

### 7. Rate of Primary Amine Grafting Functions on the Surface of the NPC₂ Nanoplatform

In a hemolysis tube, 20 μL of an aqueous solution of nanoplatforms (typically a few mg/ml) are mixed, 100 μL of an aqueous solution of KCN containing pyridine (Kit test Kaiser Aldrich), 100 μL of a solution alcoholic phenol (80% in ethanol, Kaiser Aldrich test kit) and 100 μL of an alcoholic solution of ninhydrin (6% in ethanol, Kaiser Aldrich test kit). The mixture is heated at 120° C. for 5 minutes, then allowed to cool to room temperature. 20 μL of the previously obtained solution are diluted in 2 ml of ethanol, then the absorbance at 580 nm of this new solution is measured. The concentration of primary amine reacted with ninhydrin is deduced from the absorbance value and therefore reflects the amount of primary reactive amine present on the surface of nanoplatforms. For nanoplatforms, the value obtained is 0.7 μmol of reactive NH₂ per mg of nanoplatforms. For refunctionalized nanoplatforms, the rate of primary amine grafting functions is 3.3 μmol of reactive NH2 per mg of refunctionalized nanoplatforms.

### 8. Fluorescence of the NPC₂ Nanoplatform

The absorbance of a freshly prepared aqueous aerated and thermally balanced nanoplatform solution is measured on a JASCO V-570 spectrometer at room temperature, in a quartz cell with an optical path of 1 cm. The maximum absorbance of nanoplatforms takes place at the wavelength of 360 nm. The fluorescence emission of nanoplatforms is measured on the same nanoplatform solution after prior dilution thereof until an absorbance of less than 0.15 at 360 nm is reached. The fluorescence is recorded on a Fluoro-Max fluorimeter (Horiba) and the maximum emission wavelength is 455 nm.

### 9. Stability of the NPC₂ Nanoplatform

The stability of NPC₂ nanoplatforms has been tested:
in different media (water, PBS, culture medium with fetal calf serum),
at different concentrations (25 μg/ml and 100 μg/ml)
at different pH (from 4 to 11)
at different temperatures (4° and 37° C.)

The stability of nanoplatforms is monitored thanks to the evolution of fluorescence emission under an incident wavelength of 360 nm.

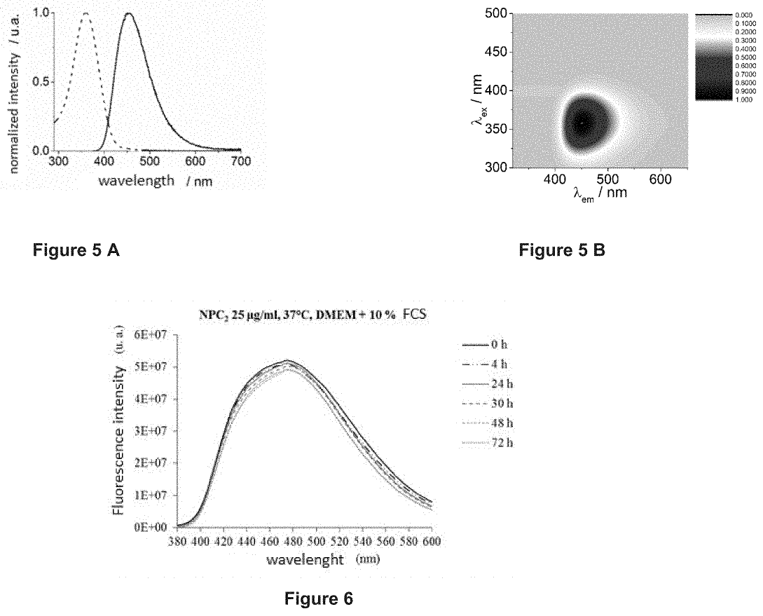

The results of FIG. 5 show a reduction in the intensity of the fluorescence emitted over time during the first 24 h before reaching a plateau after 48 h.

The examples below (examples 3 to 17) are carried out with:
fluorophore F
   5/6-carboxyfluorescein succinimidyl ester (mixed isomers), Texas Red-X succinimidyl ester (mixed isomers), Indocyanine green NHS active ester, Alexa Fluor 532/633/700/750/790 succinimidyl ester, Rhodamine B NHS active ester
the antibody A addressing agent
   Cetuximab, anti EGFR antibody or Bevacizumab anti VEGF antibody
the peptide A addressing agent
   RGD, TAT, angiopep-2
the active anti-tumor molecule M

### Paclitaxel, Docetaxel, Etoposide, Doxorubicin, Epirubicin, Idarubicin, Pirarubicin, Zorubicin, Aclarubicin,

### Example 3: Synthesis of an NPC₂ Nanoplatform with a Succinic Linker (NPC₂-L)

A solution of NPC₂ nanoplatforms in DMSO (1.0 equivalent, 50 g/l) is prepared, then succinic anhydride is added (0.5 equivalent).

The solution is stirred at room temperature overnight, then the DMSO is removed by lyophilization. Traces of DMSO are eliminated by precipitating the nanoplatforms in dichloromethane. The powder obtained is dried under vacuum. The nanoplatforms obtained are now carriers of the succinic linker.

Example 4 Activation of the NPC₂-L Succinic Linker with N-Hydroxysuccinimide (NPC₂-L-NHS)

A solution of nanoplatforms carrying the succinic linker in DMSO (1.0 equivalent, 100 g/l) is prepared, then a solution of EDC.HCl in DMSO (0.5 equivalent, 0.2 mol/l) is added. The solution is stirred at room temperature for 5 minutes and then a solution of N-hydroxysuccinimide (NHS) in DMSO (0.5 equivalent, 0.2 mol/l) is added. The solution is stirred at room temperature overnight and then dichloromethane is added, causing the precipitation of the nanoplatform. The suspension is stirred vigorously for 5 minutes, then the precipitate is recovered by centrifugation (6500 rpm, 4 minutes) then washed 3 times with dichloromethane. Residual traces of solvent are removed under reduced pressure. The powder obtained corresponds to the nanoplatform comprising the NHS activated succinic linker.

### Example 5: Synthesis of the Active NPC₂-L-M Nanoparticles

The nanoplatforms with the NHS activated succinic linker (NPC₂-L-NHS) are dissolved in a DMSO solution (1.0 equivalent of activated linker, 50 mg/l) with 1.0 equivalent of N,N′-diisopropylethylamine and 1.0 equivalent of the active anti-tumor molecule M. In the case where the active anti-tumor molecule M is in the form of the hydrochloride, 1.0 equivalent of N,N′-diisopropylethylamine is added for each equivalent of salt. This solution is stirred at room temperature overnight and then dichloromethane is added, causing the precipitation of the nanoplatform. The precipitate obtained is collected by centrifugation and then washed with dichloromethane 3 times. Once the residual solvent is removed under reduced pressure, the solid residue obtained is dissolved in a volume of water and the solution is filtered by ultracentrifugation with a 1 kDa filter under air pressure (4 bars). The solution retained by the filter is washed three times with water and then frozen and lyophilized. The powder obtained corresponds to the active nanoplatform comprising the active anti-tumor molecule M bound by a succinic linker.

### Example 6: Activation of the Antibody Addressing Agent

An aqueous solution of antibodies (6 μmol/l) containing periodate (10 mM) is stirred for 30 minutes in the dark at room temperature and then the solution is filtered by ultracentrifugation with a 1 kDa filter under compressed air pressure (4 bars). The solution retained by the filter is washed three times with distilled water and can be used as it is or can be frozen and lyophilized.

### Example 7: Synthesis of NPC₂-L-A (Antibodies) Nanoparticles

A solution of nanoplatforms carrying the succinic linker (NPC₂-L) in water is prepared (1.0 equivalent of linker, 50 mg/l), then 0.1 equivalent of hydrazine hydrate is added. The solution is heated by microwave for 90 seconds at 900 W. The solution is frozen and then lyophilized. Nanoparticles now carry hydrazide functions.

Nanoparticles carrying hydrazide functions are dissolved (1.0 equivalent of hydrazide) in an aqueous solution of activated antibody (1.0 equivalent, 6 μmol/l). The solution is stirred gently for 4 h in the dark at room temperature, then a slight excess of NaBH3CN (1.1 equivalent) is added. The solution is stirred for an additional hour in the dark at room temperature. Once the reduction reaction is complete, the aqueous solution is filtered by ultracentrifugation with a 1 kDa filter under air pressure (4 bar). The solution retained by the filter is washed 3 times with distilled water and is then frozen and lyophilized. The nanoparticles are now carriers of the addressing agent antibody A bound by a linker.

### Example 8: Synthesis of NP_{C}-L-A (Peptide) Nanoparticles

Nanoplatforms with the NHS activated succinic linker (NPC₂-L-NHS) are dissolved in a DMSO solution (1.0 equivalent of NHS activated linker, 50 mg/l) with 1.0 equivalent of N,N′-diisopropylethylamine and 0.1 equivalent of a peptide A addressing agent. This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure. The solid residue obtained corresponds to the nanoplatform carrying the peptide A addressing agent.

### Example 9: Synthesis of the Active NPC₂-(LM)-LA (Antibody) Nanoparticles

To a Solution of Nanoplatforms with the Antibody Addressing Agent (NPC₂-L-A (Antibody)) in DMSO (1.0 equivalent of free linker, 50 mg/l) is added a catalytic amount of 4-dimethylaminopyridine, 1.0 equivalent of N,N′-diisopropylethylamine and 1.0 equivalent of the active antitumor molecule M. In the case where, the active antitumor molecule M is in the form of hydrochloride, 1.0 additional equivalent of diisopropylethylamine is added for each equivalent of hydrate. The solution is stirred for 5 minutes then a slight excess of EDC.HCl in solution in DMSO (1.1 equivalent, 0.2 mol/l) is added. The solution is stirred at room temperature overnight and then dichloromethane is added, causing the precipitation of the nanoplatform. The suspension is stirred gently for 5 minutes, then the precipitate is recovered by centrifugation (6500 rpm, 4 minutes) and washed 3 times with dichloromethane.

Residual traces of solvent are removed under reduced pressure. The solid residue is dissolved in one volume of water and the solution is filtered by ultracentrifugation with a 1 kDa filter under air pressure (4 bars). The solution retained by the filter is washed 3 times with water and then frozen and lyophilized. The powder obtained corresponds to the active nanoplatform comprising the antibody addressing agent A and the active antitumor molecule M both bound by a linker.

### Example 10: Synthesis of the Active NPC₂-(L-M)-L-A (Peptide) Nanoparticles

To a solution of nanoplatforms with the peptide addressing agent (NPC₂-L-A (peptide)) in DMSO (1.0 equivalent of free linker, 50 mg/l) is added 1.0 equivalent of N,N′-diisopropylethylamine and 1.0 equivalent of the active anti-tumor molecule M. In the case where the active anti-tumor molecule M is in the form of the hydrochloride, 1.0 equivalent of diisopropylethylamine is added for each equivalent of hydrochloride. This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure. The solid residue obtained is dissolved in one volume of water and the solution is filtered by ultracentrifugation with a 1 kDa filter under air pressure (4 bars). The solution retained by the filter is washed 3 times with water and then frozen and lyophilized.

The powder obtained corresponds to the active nanoplatform comprising the addressing agent peptide A and the active anti-tumor molecule M.

### Example 11: Activation of Rhodamine B with N-Hydroxysuccinimide

A solution of Rhodamine B in DMSO (1.0 equivalent, 100 g/l) is prepared, then a solution of EDC.HCl in DMSO (1.1 equivalent, 0.2 mol/l) is added. The solution is stirred at room temperature for 5 minutes and then a solution of N-hydroxysuccinimide in DMSO (1.0 equivalent, 0.2 mol/l) is added. The solution is filtered through cotton and the filtrate is frozen and then lyophilized. The powder obtained is used as it is, and corresponds to Rhodamine B with the NFIS activated ester.

### Example 12: Synthesis of NPC₂-L-F Nanoparticles

Nanoparticles carrying hydrazide functions are dissolved (1.0 equivalent of hydrazide) in a solution of fluorophore F in DMSO (1.0 equivalent, 1 mol/l). This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure. The solid residue is dissolved in one volume of water and the aqueous solution is filtered by ultracentrifugation with a 1 kDa filter under air pressure (4 bars). The solution retained by the filter is washed 3 times with distilled water and is then frozen and lyophilized. The nanoparticles now carry the fluorophore F bound by a linker and free linker.

### Example 13: Synthesis of the Active NPC₂-(L-F)-L-M Nanoparticles

Nanoparticles carrying fluorophore F are dissolved in DMSO (1.0 equivalent of free linker, 50 mg/l), then a catalytic amount of 4-dimethylaminopyridine is added, 1.0 equivalent of A/,/V-diisopropylethylamine and 1.0 equivalent of the active anti-tumor molecule M. In the case where the active anti-tumor molecule M is in the form of the hydrochloride, 1.0 equivalent of diisopropylethylamine is added for each equivalent of hydrochloride. The solution is stirred for 5 minutes then a slight excess of EDC.HCl in solution in DMSO (1.1 equivalent, 0.2 mol/l) is added. The solution is stirred at ambient temperature overnight then dichloromethane is added in order to precipitate the nanoparticles, causing the precipitation of the nanoplatform. The suspension is stirred gently for 5 minutes, then the precipitate is recovered by centrifugation (6500 rpm, 4 minutes) and washed 3 times with dichloromethane. Residual traces of solvent are removed under reduced pressure.

The solid residue obtained is dissolved in 20 ml of water and the solution is filtered by ultracentrifugation with a 1 kDa filter under air pressure (4 bars). The solution retained by the filter is washed 3 times with water and then frozen and lyophilized. The powder obtained corresponds to the active nanoplatform comprising the fluorophore F and the active antitumor molecule M.

### Example 14: Synthesis of NPC₂-(L-F)-L-A (Antibody) Nanoparticles

Nanoparticles carrying the succinic linker and of hydrazide functions are dissolved (1.0 equivalent of hydrazide) in a solution of fluorophore F in DMSO (0.5 equivalent, 1 mol/l). This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure.

The solid residue obtained is added to an aqueous solution of activated antibody (0.5 equivalent, 6 μmol/l), then the solution is gently stirred for 4 h in the dark at room temperature A slight excess of NaBH₃CN (1.1 equivalent) is added and the solution is stirred for an additional hour in the dark at room temperature. Once the reduction reaction is complete, the aqueous solution is filtered by ultracentrifugation with a 1 kDa filter under air pressure (4 bar). The solution retained by the filter is washed 3 times with distilled water and is then frozen and lyophilized. The powder obtained corresponds to the nanoplatform comprising the fluorophore F and the antibody addressing agent A.

### Example 15: Synthesis of NPC₂-(L-F)-L-A (Peptide) Nanoparticles

To a solution of nanoparticles carrying the fluorophore F in DMSO (1.0 equivalent in free succinic linker, 100 g/l) is added a solution of EDC.HCl in DMSO (0.2 equivalent, 0.2 mol/l). The solution is stirred at room temperature for 5 minutes and then a solution of N-hydroxysuccinimide in DMSO (0.1 equivalent, 0.2 mol/l) is added. The solution is stirred at room temperature overnight and then dichloromethane is added, causing the precipitation of the nanoplatform. The suspension is stirred vigorously for 5 minutes, then the precipitate is recovered by centrifugation (6500 rpm, 4 minutes) and washed 3 times with dichloromethane. Residual traces of solvent are removed under reduced pressure.

The solid residue obtained is dissolved in DMSO (1.0 equivalent of linker-NHS, 50 mg/l) with 1.0 equivalent of N,N′-diisopropylethylamine and 1.0 equivalent of the addressing agent peptide A. This solution is stirred at room temperature overnight then dichloromethane is added, causing the precipitation of the nanoplatform. The precipitate obtained is collected by centrifugation (6500 rpm, 4 minutes) and washed with dichloromethane 3 times. The solvent residues are evaporated under reduced pressure. The residue is dissolved in one volume of water and the solution is filtered by ultracentrifugation with a 1 kDa filter under air pressure (4 bars). The solution retained by the filter is washed three times with water and then frozen and lyophilized. The powder obtained corresponds to the nanoplatform comprising the fluorophore F and the peptide A addressing agent.

### Example 16: Synthesis of Active NPC₂-(LF)-(LM)-LA (Antibody) Nanoparticles

Nanoparticles carrying hydrazide functions are dissolved (1.0 equivalent of hydrazide) in a solution of fluorophore F in DMSO (0.5 equivalent, 1 mol/l). This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure.

The solid residue obtained is dissolved in an aqueous solution of activated antibody (0.5 equivalent, 6 μmol/l), then the solution is gently stirred for 4 h in the dark at room temperature. A slight excess of NaBH3CN (1.1 equivalent) is added and the solution is stirred for an additional hour in the dark at room temperature. Once the reduction reaction is complete, the aqueous solution is filtered by ultracentrifugation with a 1 kDa filter under air pressure (4 bar). The solution retained by the filter is washed 3 times with distilled water and is then frozen and lyophilized.

The solid residue obtained is dissolved in DMSO (1.0 equivalent of free linker, 50 mg/l) with a catalytic amount of 4-dimethylaminopyridine, 1.0 equivalent of N,N′-diisopropylethylamine and 1.0 equivalent of the active antitumor molecule M. In the case where the active antitumor molecule M is in the form of hydrochloride, 1.0 equivalent of diisopropylethylamine is added for each equivalent of hydrochloride. The solution is stirred for 5 minutes then a slight excess of EDC.HCl in solution in DMSO (1.1 equivalent, 0.2 mol/l) is added. The solution is stirred at room temperature overnight and then dichloromethane is added, causing the precipitation of the nanoplatform. The suspension is stirred gently for 5 minutes, then the precipitate is recovered by centrifugation (6500 rpm, 4 minutes) and washed 3 times with dichloromethane. Residual traces of solvent are removed under reduced pressure. The solid residue is dissolved in one volume of water and the solution is filtered by ultracentrifugation with a 1 kDa filter under air pressure (4 bars).

The solution retained by the filter is 3 three times with water and then is frozen and lyophilized. The powder obtained corresponds to the active nanoplatform comprising the fluorophore F, the antibody addressing agent A and the active antitumor molecule M.

### Example 17: Synthesis of the Active NPC₂-(L-F)-(L-M)-L-A (Peptide) Nanoparticles

To a solution of nanoparticles carrying the fluorophore F in DMSO (1.0 equivalent in free succinic linker, 100 g/l) is added a solution of EDC.HCl in DMSO (1.1 equivalent, 0.2 mol/l). The solution is stirred at room temperature for 5 minutes and then a solution of N-hydroxysuccinimide in DMSO (1.0 equivalent, 0.2 mol/l) is added. The solution is stirred at room temperature overnight and then dichloromethane is added, causing the precipitation of the nanoplatform. The suspension is stirred vigorously for 5 minutes, then the precipitate is recovered by centrifugation (6500 rpm, 4 minutes) and washed 3 times with dichloromethane. Residual traces of solvent are removed under reduced pressure.

The solid residue obtained is dissolved in DMSO (1.0 equivalent of linker-NHS, 50 mg/l) with an equivalent of N,N′-diisopropylethylamine and 0.1 equivalent of the addressing agent peptide A and 0.9 equivalent of the active anti-tumor molecule M. This solution is stirred at room temperature overnight and then dichloromethane is added, causing the precipitation of the nanoplatform. The precipitate obtained is collected by centrifugation (6500 rpm, 4 minutes) and washed with dichloromethane 3 times. Solvent residues are evaporated under reduced pressure. The residue obtained is dissolved in one volume of water and the solution is filtered by ultracentrifugation with a 1 kDa filter under air pressure (4 bars). The solution retained by the filter is washed 3 times with water and then frozen and lyophilized. The powder obtained corresponds to the nanoplatform comprising the fluorophore F, the addressing agent peptide A and the active antitumor molecule M.

### Example 18: Syntheses of Active Nanoplatforms

1. Synthesis of an Active Nanoplatform with Long Linker and Paclitaxel

A solution of nanoplatforms in DMSO (50 g/l, 2 ml) is prepared, then succinic anhydride is added (9.4 mg). The solution is stirred at room temperature overnight, then the DMSO is removed by lyophilization. Traces of DMSO are eliminated by precipitating the nanoplatforms in dichloromethane. The powder obtained is dried under vacuum (m=66 mg). The nanoplatforms obtained are now carrying the linker. The nanoplatforms with the succinic linker are then dissolved in DMF (67 g/l, 0.6 ml) is added paclitaxel (6.0 mg), DMAP in catalytic amount and then EDC.HCl (6.2 mg). The reaction mixture is stirred at room temperature for 60 h, then water is added to destroy the unreacted EDC. The solvent mixture (water and DMF) is evaporated under reduced pressure, then the powder obtained is washed with acetone and is collected by centrifugation. The solid obtained is purified on a Sephadex LH20 column using water as eluent.

The aqueous phases containing the right product are combined and then evaporated under reduced pressure. The powder obtained corresponds to the active nanoplatform (m=31.7 mg) comprising the active molecule paclitaxel bound by a long linker.

2. Synthesis of an Active Nanoplatform with Long Linker and Doxorubicin

A solution of nanoplatforms carrying the long linker in DMSO (100 g/l, 1 ml) is prepared, as described above, then a solution of EDC.HCl in DMSO (0.178 mol/l, 0.27 ml) is added. The solution is stirred at room temperature for 5 minutes and then a solution of N-hydroxysuccinimide in DMSO (0.174 mol/l, 0.26 ml) is added. The solution is stirred at room temperature for 21 h then dichloromethane (12 ml) is added in order to saturate the DMSO with dichloromethane and to precipitate the active nanoplatforms. The suspension is stirred vigorously for 5 minutes, then the precipitate is recovered by centrifugation (6500 rpm, 4 minutes) then washed 3 times with dichloromethane. Residual traces of solvent are removed under reduced pressure. The powder obtained corresponds to the active nanoplatform (m=80.8 mg) comprising the NHS activated long linker. Nanoplatforms with the NHS activated long linker can be dissolved in a DMSO solution containing potassium carbonate and doxorubicin hydrochloride.

This solution is stirred at room temperature overnight and then dichloromethane can be added. The precipitate obtained is collected by centrifugation and washed with dichloromethane 3 times. Once the residual solvent is removed under reduced pressure, if the product obtained is not pure, the residue is purified on Sephadex LH20 with water as eluent. The aqueous phases containing the pure product are combined and the solvent is removed by lyophilization. The solid residue obtained corresponds to the active nanoplatform comprising the active molecule doxorubicin bound by a long linker.

3. Synthesis of an Active Nanoplatform with Fluorophore and Short Linker and Active Molecule with Long Linker.

A solution of nanoplatforms in DMF (25 μg/l, 4 ml) is prepared, then sodium carbonate is added (424 mg). The solution is heated to 61° C. and once this temperature has been reached, lissamine rhodamine B is added (1.5 mg). The solution is stirred at 61° C. overnight and the DMF is evaporated under reduced pressure then the residue obtained is neutralized with hydrochloric acid (pH 1). The aqueous solution is concentrated under reduced pressure and then the residue is dissolved in ethanol, centrifuged and the supernatant is collected in order to remove the salts which are not soluble in ethanol. Once the ethanol is removed under reduced pressure, the powder obtained (69 mg) is dried under vacuum. The nanoplatforms obtained now carry the lissamine rhodamine B fluorophore with the short linker. The nanoplatforms with the fluorophore lissamine rhodamine B (60 mg) are then dissolved in DMSO, then sodium carbonate is added (424 mg). The suspension is stirred at room temperature for 5 minutes, then succinic anhydride (100 mg) is added.

The solution is stirred at room temperature overnight and then water is added, followed by hydrochloric acid until the pH of the solution reaches pH 1. The solution is concentrated under reduced pressure and then the residual solvents are removed by lyophilization. The residue obtained is dissolved in absolute ethanol and the insoluble salts are removed by centrifugation. The supernatant alcoholic phase is collected and then evaporated under reduced pressure until a powder is obtained. Traces of residual succinic acid are removed by washing the solid residue obtained with chloroform and then with acetone. The residual powder (38 mg) is dried under vacuum. The nanoplatforms carrying the lissamine rhodamine B fluorophore with the short linker obtained are now carrying the long linker. A solution of the nanoplatforms with the fluorophore lissamine rhodamine B and the long linker in DMF (12.5 g/l, 2 ml) is prepared, then paclitaxel (3 mg), DMAP (catalytic amount) and EDC.HCl 6.18 mg) are added.

The solution is stirred at room temperature for 60 h and then water is added. The solution is concentrated under reduced pressure and the residual powder obtained is washed with acetone. Once dried, the powder obtained is purified on Sephadex LH20 using water as eluent. The aqueous phases containing the right product are collected and the water is removed by lyophilization. The powder obtained (22.9 mg) corresponds to the active nanoplatform comprising the fluorophore lissamine rhodamine B bound by a short linker and the active molecule paclitaxel bound by a long linker.

4. Synthesis of an Active Nanoplatform with Addressing Agent with Long Linker and Active Molecule with Long Linker.

A solution of nanoplatforms in DMSO (50 g/l, 2 ml) is prepared, then succinic anhydride is added (9.4 mg). The solution is stirred at room temperature overnight, then the DMSO is removed by lyophilization. Traces of DMSO are eliminated by precipitating the nanoplatforms in dichloromethane. The powder obtained is dried under vacuum (m=66 mg). The nanoplatforms obtained are now carrying the long linker. The nanoplatforms with the succinic linker are then dissolved in DMF (67 g/l, 0.6 ml). To the solution obtained is added paclitaxel (6.0 mg), DMAP in catalytic amount and then EDC.HCl (6.2 mg). The reaction mixture is stirred at room temperature for 60 h, then water is added to destroy the unreacted EDC. The solvent mixture (water and DMF) is evaporated under reduced pressure, then the powder obtained is washed with acetone and is collected by centrifugation. The solid obtained is purified on a Sephadex LH20 column using water as eluent.

The aqueous phases containing the right product are combined and then evaporated under reduced pressure. The powder obtained corresponds to the active nanoplatform (m=31.7 mg) comprising the active long linker molecule. Nanoplatforms with the active succinic linker molecule can then be dissolved in DMF, then a diester derivative of the RGD peptide and EDC. HCl are added. The reaction mixture is stirred at room temperature for 60 h, then water is added to destroy the unreacted EDC. The solvent mixture (water and DMF) is evaporated under reduced pressure, then the powder obtained is washed with acetone and is collected by centrifugation. The solid obtained is purified on a Sephadex LH20 column using water as eluent. The aqueous phases containing the right product are combined and then evaporated under reduced pressure. The powder obtained corresponds to the active nanoplatform comprising the addressing agent bound by a long linker and the active molecule bound by a long linker.

5. Synthesis of an Active Nanoplatform with Addressing Agent/Active Molecule with Long Linker and Fluorophore with Short Linker.

A solution of nanoplatforms in DMF (25 g/l, 4 ml) is prepared, then sodium carbonate is added (424 mg). The solution is heated to 61° C. and once this temperature has been reached, lissamine rhodamine B is added (1.5 mg). The solution is stirred at 61° C. overnight and the DMF is evaporated under reduced pressure. The residue obtained is neutralized with hydrochloric acid (pH˜1). The aqueous solution is concentrated under reduced pressure, then the residue is dissolved in ethanol, centrifuged and the supernatant is collected in order to remove the salts which are not soluble in ethanol. Once the ethanol is removed under reduced pressure, the powder obtained (69 mg) is dried under vacuum. The nanoplatforms obtained now carry the lissamine rhodamine B fluorophore with the short linker. The nanoplatforms with the fluorophore lissamine rhodamine B (60 mg) are then dissolved in DMSO, then sodium carbonate is added (424 mg). The suspension is stirred at room temperature for 5 minutes, then succinic anhydride (100 mg) is added.

The solution is stirred at room temperature overnight and then water is added, followed by hydrochloric acid until the pH of the solution reaches ˜1. The solution is concentrated under reduced pressure and then the residual solvents are removed by lyophilization. The residue obtained is dissolved in absolute ethanol and the insoluble salts are removed by centrifugation. The supernatant alcoholic phase is collected and then evaporated under reduced pressure until a powder is obtained. Traces of residual succinic acid are removed by washing the solid residue obtained with chloroform and then with acetone. The residual powder (38 mg) is dried under vacuum. The nanoplatforms carrying the lissamine rhodamine B fluorophore with the short linker obtained are now carrying the long linker. A solution of the nanoplatforms with the fluorophore lissamine rhodamine B and the long linker in DMF (12.5 g/l, 2 ml) is prepared, then paclitaxel (3 mg), DMAP (catalytic amount) and EDC.HCl (6.18 mg) are added.

The solution is stirred at room temperature for 60 h and then water is added. The solution is concentrated under reduced pressure and the residual powder obtained is washed with acetone, then once dried is purified on Sephadex LH20 using water as eluent. The aqueous phases containing the right product are collected and the water is removed by lyophilization. The powder obtained (22.9 mg) corresponds to the active nanoplatforms carrying the fluorophore lissamine rhodamine B (short linker) with paclitaxel (long linker). The nanoplatforms with the active long linker molecule and the short linker fluorophore are then dissolved in DMF, then a diester derivative of the RGD peptide and EDC.HCl are added. The reaction mixture is stirred at room temperature for 60 h, then water is added to destroy the unreacted EDC. The solvent mixture (water and DMF) is evaporated under reduced pressure, then the powder obtained is washed with acetone and is collected by centrifugation.

The solid obtained is purified on a Sephadex LH20 column using water as eluent. The aqueous phases containing the right product are combined and then evaporated under reduced pressure. The powder obtained corresponds to the active nanoplatform comprising the addressing agent and the active molecule each bound by a long linker and the fluorophore bound by a short linker.

### Example 19: In Vitro Study of the Safety of NPC₂

The goal is to measure the cell survival rate in the presence of a range of NPC₂ and determine the impact of these nanoplatforms on cell viability.

### Cellular Culture

All in vitro tests are performed on five cell lines:

TABLE 4
Cell lines and in vitro exoerimental conditions

| | | | |
|---|---|---|---|
| Cell line | Cancerous line of | Non-cancerous cell | Non-cancerous |
| | glioblastome | line of endothelial | cell line of |
| | U-87-MG | cells, HMEC-1 | fibroblasts |
| | | (Human | NHDF (Normal |
| | | Microvascular | Human Dermal |
| | | Endothelial Cells-1) | Fibroblasts) |
| Supplier | American Type | ATCC | Lonza |
| | Culture Collection | | |
| | (ATCC) | | |
| Culture | Eagle's Minimum | MCB1 131 (Gibco) | Fibroblast |
| medium | Essential Medium | 10% heat inactivated | growth medium |
| | (EMEM, Invitrogen) | fetal calf serum | supplemented |
| | 10% fetal calf serum | (Lonza) | with the fibro- |
| | (Lonza) | 2 mM Glutamine | blast growth kit |
| | 2 mM Glutamine | (Gibco) | (Lonza) |
| | (Gibco) | 1% (100 U/ml) | |
| | 1% (100 U/ml) | penicilline- | |
| | penicilline- | streptomycine (Gibco) | |
| | streptomycine | 10 ng/ml Endothelial | |
| | (Gibco) | Growth Factor (EGF | |
| | | human protein, Life | |
| | | technologies) | |
| Seeding | 12 500 cells/cm² | 9 400 cells/cm² | 3 500 cells/cm² |
| density | | | |
| Seeding | 24 h | 24 h | 48 h |
| time before | | | |
| treatment | | | |

| | | |
|---|---|---|
| Cell line | Non-cancerous line | Non-cancerous line |
| | of oligodendrocyte | C8-D1A |
| | OLN93 | |
| Supplier | Richter-Landsberg | ATCC |
| | and Heinrich | |
| Milieu de | Dulbecco's Modified | Dulbecco's Modified |
| culture | Eagle Medium | Eagle Medium |
| | (DMEM, Invitrogen) | (DMEM, Invitrogen) |
| | 10% fetal calf serum | 10% fetal calf serum |
| | (Lonza), | (Lonza) |
| | 2 mM glutamine, | 2 mM glutamine |
| | (Gibco) | (Gibco) |
| | 1% (100 U/ml) de | 1% (100 U/ml) de |
| | penicilline- | penicilline- |
| | streptomycine. | streptomycine |
| | (Gibco) | (Gibco) |
| Seeding | 37 500 cells/cm² | 15 600 cells/cm² |
| density | | |
| Seeding | 24 h | 24 h |
| time before | | |
| treatment | | |

### Survival Test—Treatment

Following the seeding of the cells as described above, the adherent cells are treated with a range of concentrations (1, 5, 25, 50, 75 and 100 μg/ml) of NPC₂ dispersed in the culture medium. The total volume is maintained at 200 μL per well and each condition is carried out in quadruplet (impedance test) and/or quintuplet (colorimetric test). A cell viability test is carried out either by a colorimetric test after 72 h of treatment, or by an impedance test over a period of at least 72 h. All the colorimetric tests were repeated 3 times for each line, while the impedance test was repeated 2 times on the U-87-MG and HMEC-1 lines and was done once on the NDHF line.

### (1) Colorimetric Test—MTT Test

This test uses as reagent 3-(4,5-Dimethylthiazol-2-yl)-2,5-Diphenyltetrazolium Bromide (MTT), which is converted into formazan (crystal violet) by living cells. The latter absorbent at the wavelength of 600 nm is detectable by an absorbance measurement.

The cells, after having undergone the above treatment with NPC₂, are incubated with 200 μL of MTT reagent at 0.5 mg/ml dispersed in the culture medium (Correard, F., et al. (2014). International Journal of Nanomedicine 9: 5415-5430) for 3 to 4 h at 37° C. As soon as the formazan crystals are visible in the cells under a light field microscope, the medium is replaced by 200 μl of dimethyl sulfoxide (DMSO). The cells are then lysed, the formazan crystals dissolved and the absorbance is measured with a Multiskan Spectrophotometer (Ascent, Labtec Systems) with a filter at 600 nm.

The percentage of viability is obtained by comparing the absorbance value of the treated cells with the absorbance value of the untreated control cells, which is considered as a reference and equivalent to 100% of viability. The standard deviation is calculated from 3 independent experiments.

### (2) Colorimetric Test—Alamar Blue Test

This test is done in one step and uses a reagent based on resazurin, which is reduced by living cells. This results in a shift in the reagent absorbance, the absorbance peak of which goes from 570 nm (oxidized form) to 595 nm (reduced form) detectable by an absorbance measurement.

The cells, after having undergone the above treatment with NPC₂, are incubated with 20 μL of Alamar Blue reagent; or 10% of the volume of medium (Pasquier, E., et al. (2013). Angiogenesis. 2013 April; 16 (2): 373-86) for 4 to 5 h at 37° C. The absorbance is then measured with a POLARstar Omega microplate reader (BMG LABTECH) with a filter at 570 nm and a filter at 595 nm.

The percentage of viability is obtained by comparing the rate of fluorescence emitted by the reduced Alamar Blue reagent in the treated cells with the rate present in the untreated control cells, which is considered as a reference and equivalent to 100% of viability. The standard deviation is calculated from 3 independent experiments.

### (3) Colorimetric Test—Sulforhodamine B Test

This test is based on protein biomass and uses an anionic sulforhodamine B probe, which strongly binds to proteins. The inhibition of cell growth is measured using a sulforhodamine B assay kit (Sigma Aldrich) as described previously in: Berges, R., et al. (2016). Mol Cancer Ther. 2016 November; 15 (11): 2740-2749).

The cells, after having been treated as above with NPC₂, are fixed at 4° C. with 50 μL of cold 10% trichloroacetic acid (TCA). The cells are then washed several times with deionized water to remove any trace of medium and/or TCA. The cells are then dried to remove all traces of water and 50 μL of sulforhodamine B are added to each well. The cells are incubated for 30 min, then rinsed 4-5 times with 10% acetic acid and dried in the open air. A volume of 200 μL of Tris base at 10 mM is added to each well and the microplate is shaken lightly to homogenize the anionic sulforhodamine B probe. The absorbance is then measured with a POLARstar Omega microplate reader (BMG LABTECH) with a filter at 565 nm and a filter at 620 nm to eliminate background noise. In order to avoid parasitic interactions between the probe and the plastic, “blanks” are produced from wells which have not undergone any treatment.

This can also be used to ensure the correct washing of cells.

The percentage of cell viability is obtained by comparing the absorbance bound to the treated cells with the absorbance bound to the untreated control cells, which is considered as a reference and equivalent to 100% of viability. The standard deviation is calculated from 3 independent experiments.

### (4) Real-Time Impedance Test

The impedance test is based on a real-time measurement of the resistance induced by a cell monolayer grown on a gold electrode, which shows variation when cells detach from the surface. The impedance measurement is carried out with an impedance meter (Xcelligence, ACEA Biosciences), and makes it possible to obtain quantitative information on the state of living cells (adhesion, proliferation, mortality) continuously and in real time.

A 96-well plate, covered with gold electrodes (E-plate 96) is seeded as described above (see Table Z) in a volume of 100 μL. When the impedance measurement reaches a plateau, the plate is removed from the counter, the culture medium is removed and the cells are treated with NPC₂ as described above. The plate is then reinserted into the reader and the impedance is measured every 15 minutes for at least 72 h.

The percentage of cell viability is obtained by comparing the measurement of impedance of the treated cells with the measurement of impedance of the untreated control cells, which is considered as a reference and equivalent to 100% of viability. The standard deviation is calculated from the number of independent experiments carried out for each cell line considered.

### Results—FIGS. [7-8]

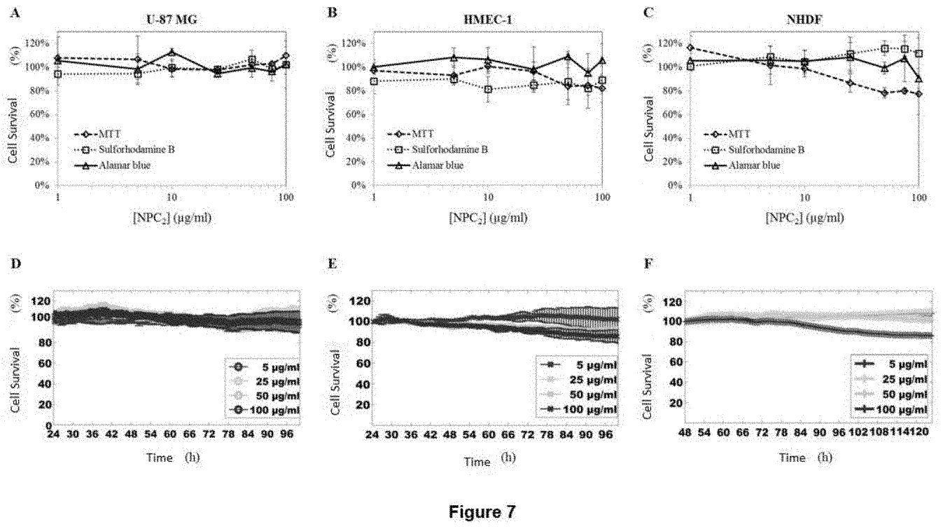

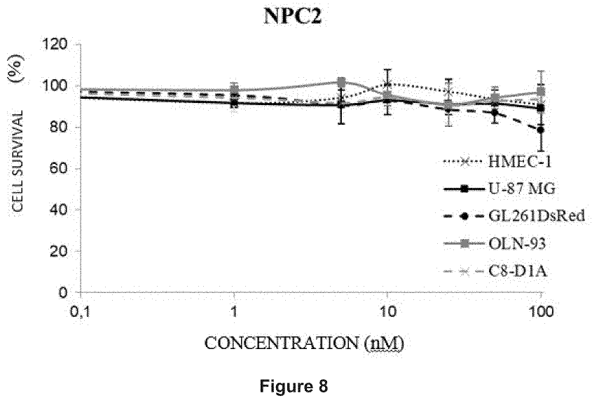

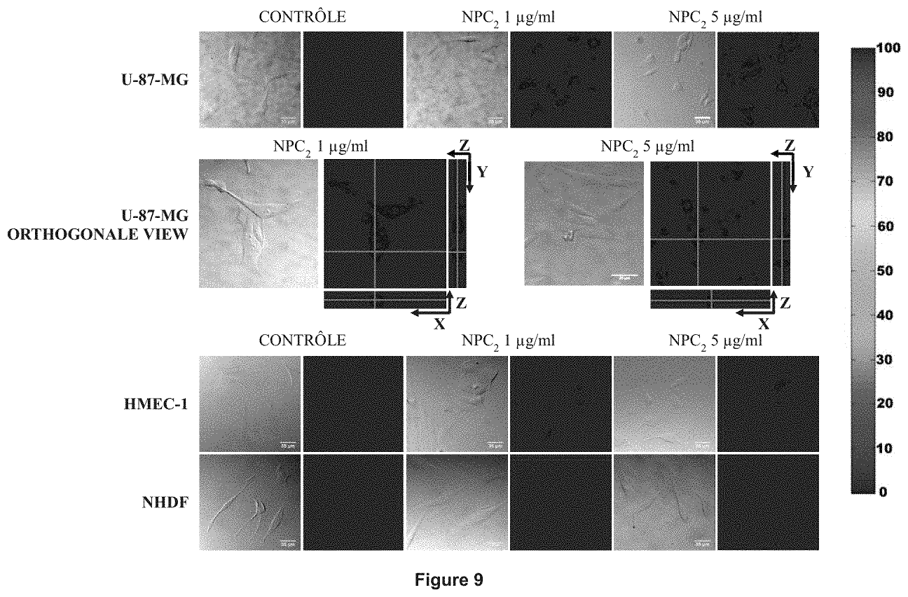

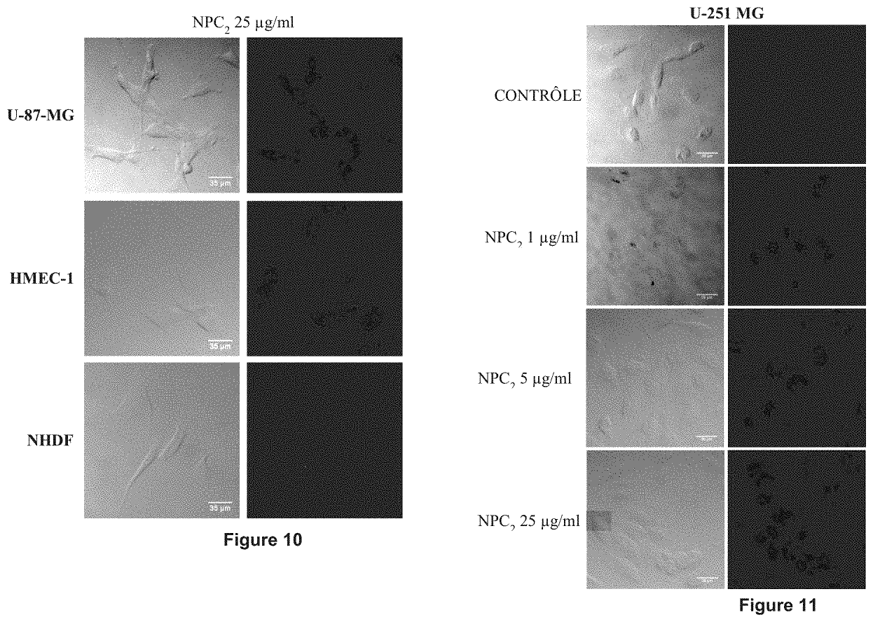

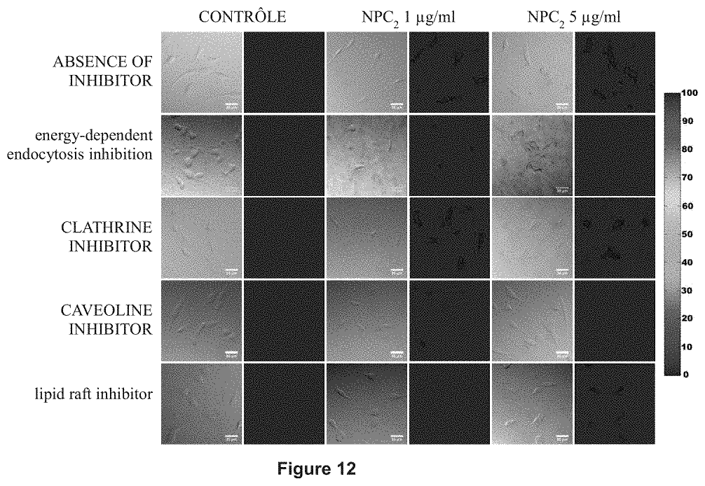

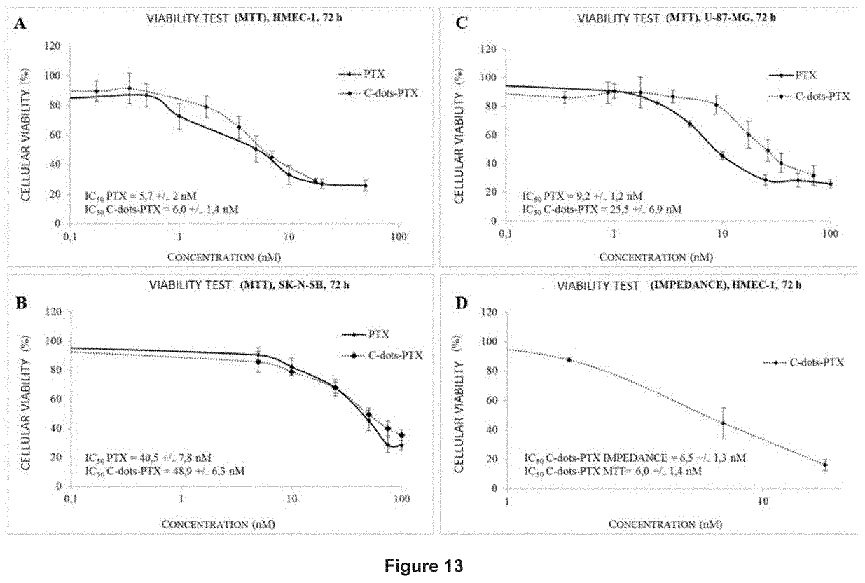

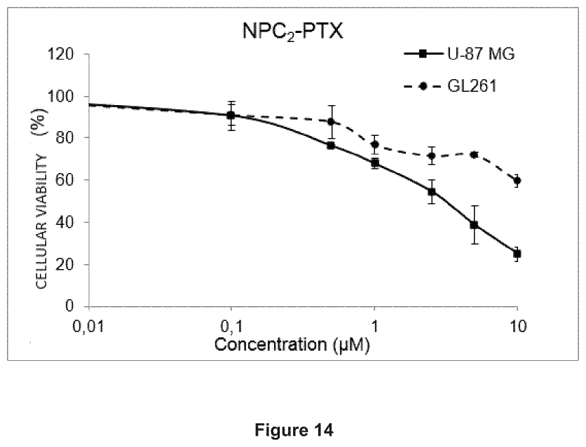

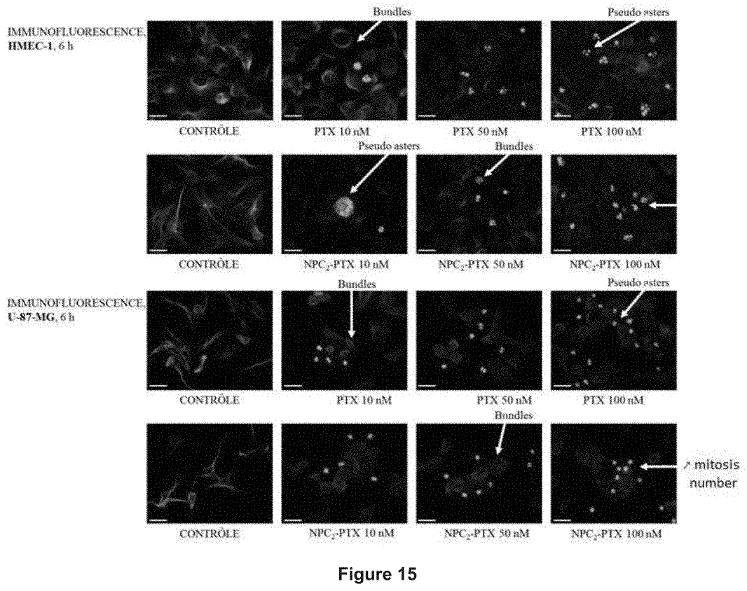

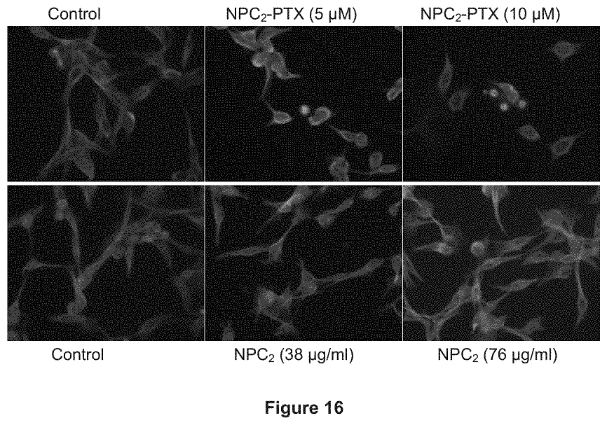

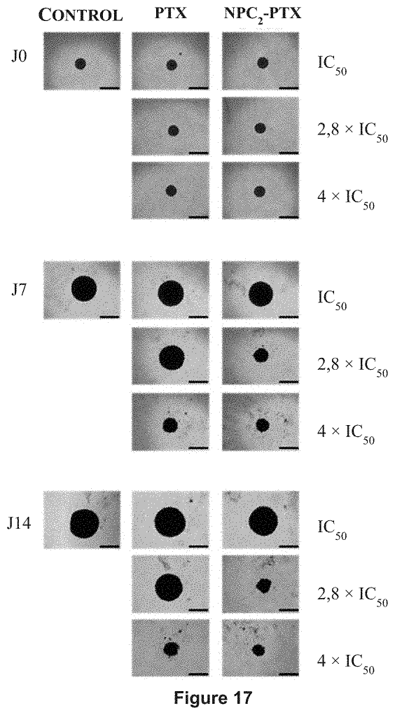

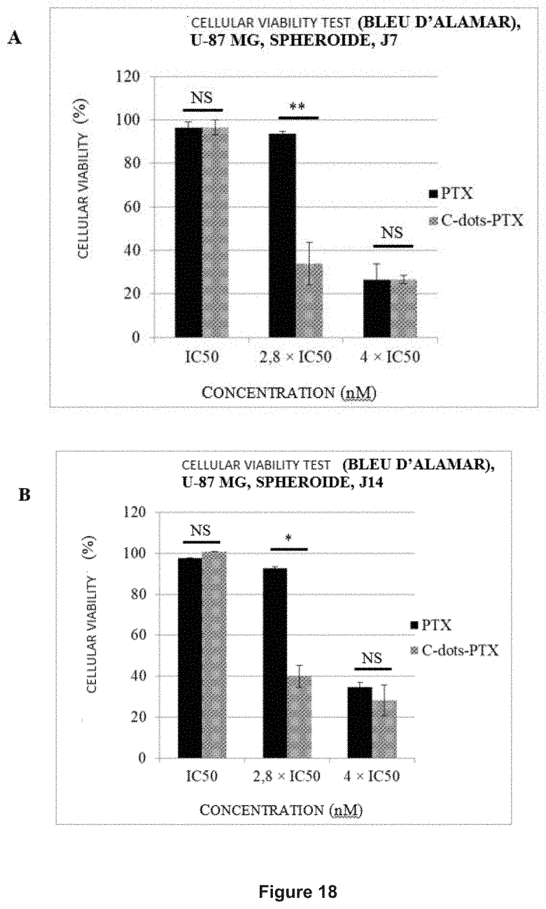

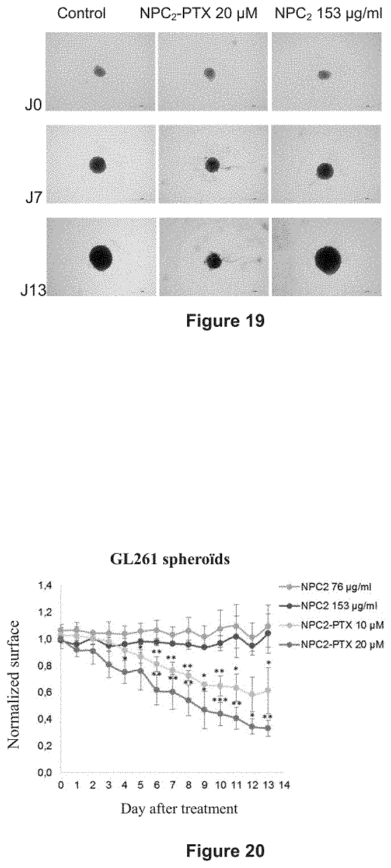

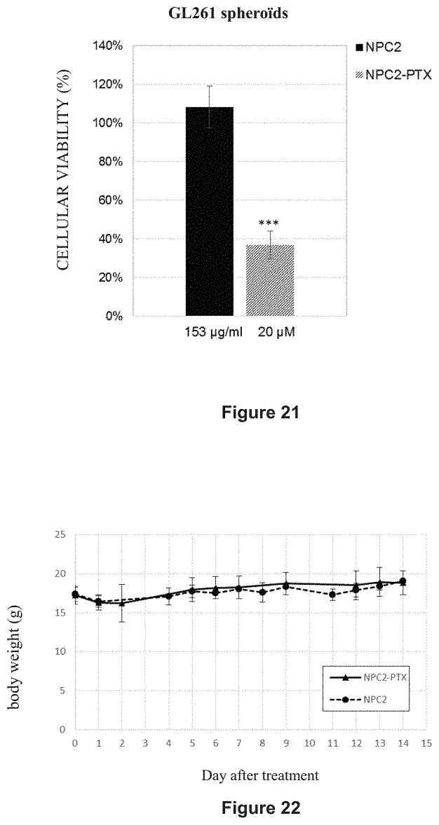

Cell viability was measured after incubation of cells with increasing concentrations of NPC₂ from 1 to 100 μg/ml for at least 72 h. U-87 MG cells show no reduction in cell viability even at the maximum concentration of 100 μg/ml (FIG. 7A, D), and whatever the test considered, the same results are observed on the OLN 93 lines. and C8-D1A (FIG. 8). HMEC-1 cells show a slight 20% decrease in cell viability at concentrations above 50 μg/ml with the MTT test (FIG. 7B), which is also observed at concentrations above 25 μg/ml with the impedance test (FIG. 7E). For NHDF cells, a 20% decrease in cell viability at concentrations above 25 μg/ml is observed with the MTT test and to 100 μg/ml with the impedance test (FIG. 7C, F), whereas ‘No significant inhibition of cell viability is measured with the Alamar Blue test (FIG. 7C).

Regarding the sulforhodamine B test, no decrease in cell viability is measured whatever the cell line considered (FIGS. 7 A, B and C).

In general, the maximum decrease in cell viability observed is 20% for the maximum concentration of 100 μg/ml of NPC₂ after 72 h of treatment. This very satisfactorily demonstrates the low toxicity of these nanoplatforms vis-à-vis the cancerous and/or non-cancerous human cell lines tested.

### Example 20: In Vitro Study of Cell Internalization

The aim is to demonstrate that there is cellular internalization of NPC₂.

### Cellular Culture

See Example [19] table [4]

U-251 MG cells are also used. They are transfected with dsRed (Alves, I. D., et al. (2014). Biochimica and Biophysica Acta (BBA)-Biomembranes 1838 (8): 2087-2098) and cultivated in:
Dulbecco's Modified Eagle Medium (DMEM, Invitrogen) with phenol red,
2 mM glutamine,
10% of fetal calf serum, and
1% (100 U/ml) of penicillin-streptomycin.

They are seeded 24 hours before treatment, at a density of 20,000 cells/cm².

### Cell Internalization Test

### (1) Treatment

After having seeded the cells U-87-MG, U251-MG, HMEC-1 and NHDF as described above, the culture medium is replaced by culture medium containing NPC₂ (1, 5 and/or 25 μg/ml in a volume equivalent to that of sowing). The cells are then incubated for 4 h at 37° C. before the medium is removed and the cells fixed in order to carry out 2-photon microscopy. This experiment is repeated 3 times with U-87-MG cells and 2 times with U-251 MG, HMEC1 and NHDF cells. Rehearsals are performed with different cell passages

### (2) Fixation

The cells are washed several times with Phosphate Buffer Saline (PBS), fixed with 4% paraformaldehyde in PBS (15 min at room temperature) and rinsed 2 times with PBS. The fixed cells are stored at 4° C. in PBS.

### (3) 2-Photon Microscopy

The cells are imaged with a Zeiss 2-photon microscope coupled to a pulsed MAI-TAI laser, whose excitation wavelength is 740 nm (λₑₓ=740 nm), and a photomultiplier capable of detecting lengths of emission wave going from 480 nm to 550 nm (480 nm<λₑₘ<550 nm). A 20× dry lens is used. The mean intensity of fluorescence and the standard deviation of the control cells not treated with NPC₂ are used to determine the threshold of autofluorescence contribution in cells treated with NPC₂ using MATLAB. Only fluorescence intensities greater than at least once the standard deviation of the average control intensity are displayed using MATLAB.

### Results—FIGS. [9; 10; 11]

The safety of NPC₂ having been demonstrated, it is necessary to ensure that they are well internalized by glioblastoma cells for their future therapeutic applications. For this, the experimentation protocol described above was implemented and once the fluorescence correction applied to the treated cells it appears that:
After 4 h of incubation with NPC₂, there is a significant internalization of the latter by U-87-MG cells at concentrations of 1 and 5 μg/ml, while it is negligible with HMEC-1 cells and NHDF (FIG. 9).
At a concentration of 25 μg/ml, the U-87-MG cells still exhibit excellent internalization of NPC2 and this in a homogeneous manner. At this same concentration, the NFIDF cells do not internalize the NPC₂ whereas the FIMEC-1 cells show a slight increase in the internalization of the NPC₂ but not in a uniform manner (FIG. 10).
The cellular localization of NPC₂ within U-87-MG cells was also observed by carrying out a Z-scanning of the cells. The fluorescence images along the XY axis and the corresponding XZ and YZ orthogonal sections show a homogeneous distribution of NPC₂ along the Z axis (FIG. 9, orthogonal views of U-87-MG cells).
The results obtained with the additional human glioblastoma line U-251 MG also show a massive internalization of NPC₂ after 4 h of incubation at all concentrations (1, 5 and 25 μg/ml of NPC₂) (FIG. 11) unlike FIMEC-1 and NFIDF cell lines.

Furthermore, it has been observed that 4 h of incubation of NPC₂ is the optimal duration of internalisation by U-87-MG cells compared to a short incubation of 2 h and a prolonged incubation of 24 h. Here, it is also shown that NPC₂ are preferentially internalized in cancer cells.

### Example 21: Determination of the Channel Used for Cellular Internalization of NPC₂

The goal is to determine the cellular mechanism used.

### Cellular Culture

Only U-87-MG cells are used: cf. Example [19], table [4], column U-87-MG.

### Cell Internalization Test

See example [20].

### Inhibition of Energy-Dependent Internalization

After having seeded the U-87-MG cells as described above, the cells are treated with 1 and/or 5 μg/ml of NPC₂ and incubated for 4 h at 4° C. then rinsed with PBS and fixed in order to carry out the observation by 2-photon microscopy (cf. Example [20]).

### Inhibition of Different Endocytosis Pathways

After having seeded the U-87-MG cells as described above, the cells are treated with:
5 μg/ml of chlorpromazine (clathrin inhibitor), or
200 μM genistein (caveolin inhibitor), or
1 mM methyl-cyclodextrin (lipid raft inhibitor).

After 30 min of incubation at 37° C., the cells are washed with cold PBS and then treated with 1 and/or 5 μg/ml of NPC₂ for 4 h at 37° C. while the control cells are incubated with medium alone. The cells are then rinsed with PBS and fixed for 2-photon microscopy (cf. Example [20]). These experiments are carried out 2 times with different cell passages.

### Results—FIG. [12]

In order to define the mechanism implemented by the cells for the internalization of the NPC₂s, the U-87-MG cells were incubated with the NPC₂s at 4° C. At this temperature, the internalization of NPC₂ is negligible, which suggests an energy-dependent internalization mechanism (FIG. 12, line 2). U-87-MG cells were also treated with chlorpromazine (clathrin inhibitor), or genistein (caveolin inhibitor), or methyl-cyclodextrin (lipid raft inhibitor). It was then observed that inhibiting clathrin has no pronounced effect on the internalization of NPC₂, while inhibiting caveolin or the lipid raft pathway drastically reduces it (FIG. 12, lines 3-5).

These observations therefore suggest that the internalization mechanism of NPC₂ mainly uses the endocytosis pathways bound to caveolin and lipid rafts. It could also indicate an effect of the “protein crown” which is created on the surface of NPC₂, which would bind either to caveolin or to the lipid rafts present on the surface of glioblastoma cells. Indeed, it has been shown that this “protein crown” promotes the internalization of gold nanoparticles with a diameter of less than 20 nm by a caveolin-dependent mechanism, while the larger nanoparticles are internalized by a clathrin-dependent mechanism (Cheng, X., et al. (2015)). ACS Applied Materials & Interfaces 7 (37): 20568-20575). Consequently, and in view of these results, it is deduced therefrom that the “protein crown” in combination with the size of the NPC₂ preferentially promotes internalization bound to caveolin by the glioblastoma cells.

### Example 22: In Vitro Study of Cytotoxicity Induced by PTX and NPC₂-PTX

The aim is to measure the cell survival rate in the presence of a range of PTX or a range of NPC₂-PTX and to determine the IC₅₀ corresponding to the concentration of PTX or NPC₂-PTX inhibiting 50% of the cellular viability.

### Cellular Culture

All the in vitro tests are carried out on three cell lines: cf. table below.

| | | | |
|---|---|---|---|
| Cell line | Lignée cancéreuse | Non-cancerous cell | Cancerous |
| | de glioblastome | line of endotheliales | cell of |
| | U-87-MG | cells HMEC-1 | neuroblastome |
| | | (Human Micro- | SK—N—SH |
| | | vascular Endothelial | |
| | | Cells-1) | |
| Supplier | American Type | ATCC | ATCC |
| | Culture Collection | | |
| | (ATCC) | | |
| Culture | Eagle's Minimum | MCB1 131 (Gibco) | RPMI-1640 |
| medium | Essential Medium | 10% heat inactivated | (Lonza) |
| | (EMEM, | fetal calf serum | 10% fetal calf |
| | Invitrogen) | (Lonza) | serum (Lonza) |
| | 10% fetal calf | 2 mM Glutamine | 2 mM Glutamine |
| | serum (Lonza) | (Gibco) | (Gibco) |
| | 2 mM Glutamine | 1% (100 U/ml) | 1% (100 U/ml) |
| | (Gibco) | penicilline-strepto | penicilline- |
| | 1% (100 U/ml) | mycine (Gibco) | streptomycine |
| | penicilline- | 10 ng/ml Endothelial | (Gibco) |
| | streptomycine | Growth Factor (EGF | |
| | (Gibco) | human protein, Life | |
| | | technologies) | |
| Seeding | 12 500 cells/cm² | 9 400 cells/cm² | 33 000 cells/cm² |
| density | | | |
| Seeding | 24 h | 24 h | 24 h |
| time before | | | |
| treatment | | | |

### Table 5. Cell Lines and In Vitro Experimental Conditions

### Survival Test

### (1) MTT Treatment & Test

Following the seeding of the cells as described above, the adherent cells are treated with a concentration range either of paclitaxel (PTX), or of paclitaxel coupled to NPC₂ (NPC₂-PTX) as follows:
U-87 MG are treated with a range of PTX ranging from 1 to 100 nM and a range of NPC₂-PTX ranging from 0.35 to 70 nM (PTX equivalent);
HMEC-1 are treated with a range of PTX ranging from 0.05 to 50 nM and a range of NPC₂-PTX ranging from 0.0175 to 17.5 nM (PTX equivalent); and
SK-N-SH are treated by a range of PTX going from 5 to 100 nM and a range of NPC₂-PTX going from 5 to 100 nM (equivalent PTX).

The volume of seeding and treatment is maintained at 150 μL per well and each condition is carried out with a conventional incubation at 37° C. A cell viability test is carried out by an MTT test after 72 h of treatment. This test uses as reagent 3-(4,5-Dimethylthiazol-2-yl)-2,5-Diphenyltetrazolium Bromide (MTT), which is converted into formazan (crystal violet) by living cells. The latter absorbent at the wavelength of 600 nm is detectable by an absorbance measurement.

The cells, after having undergone the above treatment with PTX and/or with NPC₂-PTX, are treated with 150 μL of MTT reagent at 0.5 mg/ml dispersed in the culture medium (Correard, F., et al. (2014). International Journal of Nanomedicine 9: 5415-5430) for 3 to 5 h at 37° C. As soon as the formazan crystals are visible in the cells under a light field microscope, the medium is replaced by 150 μl of dimethylsulfoxide (DMSO). The cells are then lysed, the formazan crystals dissolved, and the absorbance is measured with a Multiskan spectrophotometer (Ascent, Labtec Systems with a filter at 600 nm.

The percentage of viability is obtained by comparing the absorbance value of the treated cells with the absorbance value of the untreated control cells, which is considered as a reference and equivalent to 100% of viability. The standard deviation is calculated from 3 independent experiments.

### (2) Real-Time Impedance Treatment & Test

This test, performed only with the HMEC-1 cell line, is based on a real-time measurement of the resistance induced by a cell monolayer cultured on a gold electrode, which will show a variation when the cells detach from the surface. The impedance measurement is carried out with the Real Time Cell laAnalyser system (RTCA, ACEA Biosciences), and makes it possible to obtain quantitative information on the biological state of the cells (adhesion, proliferation, mortality) continuously and in real time. The seeding of a 96-well plate, covered with gold electrodes (E-plate 96) is carried out as described previously (cf. table 5). When the impedance measurement reaches a plateau, the plate is removed from the RTCA station, the culture medium is removed, and the cells are treated with a range of NPC₂-PTX ranging from 0.35 to 17.5 nM (equivalent PTX). The plate is then reinserted in the station and the impedance is measured every 15 minutes for at least 72 h.

The percentage of cell viability is obtained by comparing the measurement of impedance of the treated cells with the measurement of impedance of the untreated control cells, which is considered as a reference and equivalent to 100% of viability. The standard deviation is calculated from two independent experiments.

### Results—FIG. [13]

The cell viability tests were carried out by two techniques: an MTT test then an impedance test in order to confirm the absence of interaction between the NPC₂-PTX and the colorimetric reagent used (MTT) for the measurement of cell survival.

The results obtained with the MTT tests are summarized in the table below:

TABLE 6
MTT test results

| Cell line | U-87 MG | HMEC-1 | SK-N-SH |
|---|---|---|---|
| IC₅₀ PTX (nM) | 9.2 ± 1.2 | 5.7 ± 2.0 | 40.5 ± 7.8 |
| IC₅₀ NPC₂-PTX (nM) | 25.5 ± 6.9 | 6.0 ± 1.4 | 48.9 ± 6.3 |
| significance | p = 0.0053 | p = 0.96 | p = 0.22 |

In summary, a similar activity of PTX and NPC₂-PTX is shown on the HMEC-1 and SK-N-SH lines, while for the U-87 MG line the PTX is significantly more active than the NPC₂-PTX. This difference in activity between PTX and NPC₂-PTX on U-87 MG could be due to a difference in intracellular penetration mechanism between PTX and NPC₂-PTX. For SK-N-SH, it should be noted that higher concentrations of PTX and NPC₂-PTX are necessary to reach IC₅₀, compared to the other two cell lines.

The impedance test is performed on HMEC-1 cells (FIG. 12D). The results obtained for NPC₂-PTX with this method (IC₅₀=6.5±1.3 nM) are comparable to the results obtained with the MTT test (IC₅₀=6.0±1.4 nM, p=0.56). This confirms the absence of interaction with the colorimetric reagent and validates the results obtained by the tests using MTT.

### Example 23: In Vitro Study of Cytotoxicity Induced by PTX and NPC₂-PTX

The aim is to measure the cell survival rate in the presence of a range of PTX or a range of NPC₂-PTX

### Cellular Culture

All the in vitro tests are carried out on two cell lines: See table below.

TABLE 7
Cell lines and in vitro experimental conditions

| | | |
|---|---|---|
| Cell line | Cancerous line of | Cancerous line of |
| | human glioblastome | murine glioblastome |
| | U-87-MG | GL261 |
| Supplier | American Type | National Cancer |
| | Collection (ATCC) | Culture Institute, |
| | | Charles River Labs |
| Culture | Eagle's Minimum | Dulbecco's Modified |
| medium | Essential Medium | Eagle's Medium |
| | (EMEM, Inyitrogen) | (DMEM, Life |
| | 10% fetal calf | Technologies) |
| | serum (Lonza) | 10% fetal calf serum |
| | 2 mm Glutamine | 1% (100 U/ml) |
| | (Gibco) | penicillin |
| | 1% (100 U/ml) | streptomycin |
| | penicilline- | (Gibco) |
| | streptomycine | |
| | (Gibco) | |
| Seeding density | 12 500 cells/cm² | 12 500 cells/cm² |
| Seeding time | 24 h | 24 h |
| before | | |
| treatment | | |

### Survival Test—Treatment

Following cell seeding (human glioblastoma, U-87 MG and murine glioblastoma, GL261), adherent cells are treated with a concentration range of paclitaxel coupled to NPC₂ (NPC₂—PTX) from 0.1 to 10 μM (PTX equivalent). The volume of seeding and treatment is maintained at 150 μL per well and each condition is carried out with a conventional incubation at 37° C. and 5% CO₂. A cell viability test is carried out by an MTT test after 72 h of treatment.

### MTT Test

This test uses as reagent 3-(4,5-Dimethylthiazol-2-yl)-2,5-Diphenyltetrazolium Bromide (MTT), which is converted into formazan (crystal violet) by living cells. The latter detectable by an absorbance measurement at 600 nm.

The cells, after having undergone the above treatment with PTX and/or with NPC₂-PTX, are treated with 150 μL of MTT reagent at 0.5 mg/ml dispersed in the culture medium. As soon as the formazan crystals are visible in the cells under a light field microscope, the medium is replaced by 150 μl of dimethylsulfoxide (DMSO). The cells are then lysed, the formazan crystals dissolved, and the absorbance is measured with a Multiskan spectrophotometer (Ascent, Labtec Systems with a filter at 600 nm. The percentage of viability is obtained by comparing the absorbance value of the treated cells with the absorbance value of the untreated control cells, which is considered as a reference and equivalent to 100% of viability. The standard deviation is calculated from 3 independent experiments.

Results—FIG. 14 The activity of NPC₂-PTX is dose dependent and achieves a maximum effect of 40 and 80% inhibition of cell survival at the highest doses, for GL261 and U-87 MG respectively (FIG. 14).

### Example 24: Cellular Study by Immunofluorescence in Comparison with PTX

Aim: to visualize the microtubular network, target of PTX, in order to search for the pharmacological effects induced by NPC₂-PTX, in comparison with PTX.

### Cellular Culture

Only the U-87-MG and HMEC-1 lines are used (see Table 5).

### Immunofluorescence

The U-87-MG and HMEC-1 cells are seeded on Lab-Tek® culture chambers (Nunc). After 24 h of incubation, the medium is replaced by different concentrations of NPC₂-PTX or PTX (10, 50 and 100 nM). After 6 h of treatment, the medium is replaced by a solution of ice-cold methanol making it possible to fix and permeabilize the cells. After saturation with a 1% PBS-BSA (bovine serum albumin) solution, the cells are incubated for 1 h at 37° C. with a mouse anti-tubulin antibody (clone DM1A, Sigma) diluted in a solution of PBS-1% BSA, then 1 h at 37° C. in the dark with a secondary anti-mouse antibody coupled to FITC (Cell Signaling Technology) diluted in the 1% PBS-BSA solution. The labeling of the nucleus is carried out with a solution of DAPI or 4,6-diamidino-2-phenylindole (0.25 μg/ml; Sigma) for 2 min. The blade is finally mounted with Prolong® (Invitrogen) antifading. The observation is carried out with an epifluorescence microscope (Leica DM-IRBE) coupled with a digital camera (Princeton Instruments; CCD camera coolsnap FX).

### Results—FIG. [15]

By indirect immunofluorescence using an anti-tubulin antibody and labeling of the nuclei with DAPI (FIG. 15), a well microtubular network is observed at the level of the control cells, both U-87-MG and HMEC. spread out, elongated cells, a standard interphasic nucleus and some cells in mitosis. After treatment with PTX (FIGS. 15-A and 15-C), at the 3 concentrations tested, the cells are rounded, the microtubular network is disturbed. There is formation of bundles (association of microtubules) in the interphasic cells and abnormal mitotic spindles in the form of pseudo-asters in the mitotic cells. An increase in the number of mitoses is also observed. These effects are characteristic of treatment with taxane.

After treatment with NPC₂-PTX (FIG. 15-B, 15-D), there are effects comparable to those obtained after treatment with PTX at all the concentrations tested. It is observed rounded cells, bundles as well as pseudo asters. The number of mitoses is also increased compared to control.

Thus, all of the specific cytotoxic effects of PTX are found after treatment of cells in 2D culture with NPC₂-PTX, which confirms that PTX is released from its binding to NPC₂ to be pharmacologically active.

### Example 25: Cellular Study by Immunofluorescence in Comparison with NPC₂

Aim: to visualize the microtubular network, target of PTX, in order to search for the pharmacological effects induced by NPC₂-PTX, in comparison with NPC₂.

### Cell Culture

Only the U-87-MG line was used (see Table 5).

### Immunofluorescence

The U-87-MG line is used. U-87-MG cells are seeded on Lab-Tek® culture chambers (Nunc). After 24 h of incubation, the medium is replaced by medium alone (control cells) or by different concentrations of NPC₂ (38 and 76 μg/ml) or NPC₂-PTX (38 and 76 μg/ml equivalent of NPC₂, 5 and 10 mM PTX equivalent). After 24 h of treatment, the medium is replaced by a solution of ice-cold methanol making it possible to fix and permeabilize the cells. After saturation with a 1% PBS-BSA (bovine serum albumin) solution, the cells are incubated for 1 h at 37° C. with a mouse anti-b-tubulin antibody (clone DM1 A, Sigma) diluted in a PBS solution-BSA 1%, then 1 h at 37° C. in the dark with a secondary anti-mouse antibody coupled to FITC (Cell Signaling Technology) diluted in the solution of PBS-BSA 1%. The labeling of the nucleus is carried out with a solution of DAPI or 4,6-diamidino-2-phenylindole (0.25 μg/ml; Sigma) for 2 min. The slide is finally mounted with a Prolong® anti-fluorescence agent (Invitrogen).

The observation is carried out with an epifluorescence microscope (Leica DM-IRBE) coupled with a digital camera (Princeton Instruments; CCD camera coolsnap FX).

### Results—FIG. [16]

By indirect immunofluorescence using an anti-b-tubulin antibody and labeling of nuclei with DAPI, it is observed at the level of the control cells and the cells treated with NPC₂, a well spread microtubular network, elongated cells, a standard interphasic nucleus and some cells in mitosis. After treatment with NPC₂-PTX, there are effects comparable to those obtained after treatment with PTX alone at all the concentrations tested. Round cells, bundles of microtubules and anomalies in the formation of mitotic poles are observed. The number of mitoses is also increased compared to control.

### Example 26: Cytotoxicity Study in 3D Culture

Aim: to observe changes in size of the spheroids induced by PTX or NPC₂-PTX, compared to the control, and to measure the cell survival rate on cells in 3D culture in the presence of a range of concentrations of PTX or NPC₂-PTX.

### 3D Cell Culture

Spheroids (cells in 3D culture) help to better represent the tumor environment. They are composed of necrotic cells in their center and proliferating cells in the periphery. The application of NPC₂-PTX on these spheroids allows them to be studied over a longer time compared to the 2D study. The U-87 MG cells are cultured in EMEM medium supplemented with 10% of FCS, 2 mM of L-glutamine, 100 U/ml of penicillin-streptomycin and 20% (m/v) of methylcellulose. The cells are seeded on a round bottom 96-well culture plate, at the concentration of 1000 cells/well, 72 h before treatment. The cells are maintained at 37° C. and 5% of CO₂ and 10 μl of fresh medium are added every two days to all the wells (controls and treated).

### Treatment

The cells are incubated in culture medium (control cells) or containing PTX (9, 25 and 40 nM) or NPC₂-PTX (25, 70, 100 nM). These concentrations correspond to the IC50 calculated in 2D (equitoxic doses), 2.8 times the IC₅₀ and 4 times the IC₅₀ Treatment monitoring is done on the day of treatment (day), 7 days (day 7) and/or 14 days (d14) after treatment.

### (1) Optical Microscopy

Before treatment and then every two days, the spheroids are observed under an optical microscope coupled to a camera (Nikon ECLIPSE TS100), at magnification*4.;

### (2) Colorimetric Test—Alamar Blue Test

This measurement is done in one step and uses a reagent based on resazurin, which is reduced by living cells. The reduced compound (=resofurin) is detectable by fluorescence (λₑₓ=584 nm and λₑₘ=620 nm) and the signal measured is proportional to the number of living cells. Once the above treatment has been set up, 10 μL of Alamar Blue are added to each well 7 days or 14 days after treatment and the cells are incubated 12 h before the fluorescence measurement. The data are obtained by reading the fluorescence with POLARstar Omega (BMG LABETCH) after excitation at 584 nm and reading the emission at 620 nm. The percentage of viability is obtained by comparing the fluorescence rate emitted by the reduced Alamar Blue reagent in the treated cells with the rate present in the untreated control cells, which is considered as a reference and equivalent to 100% viability.

The standard deviation is calculated from 3 independent experiments.; Results—FIG. [17; 18]; (1) The concentrations of PTX and NPC₂-PTX used on the spheroids are expressed as a multiple of the IC₅₀ obtained in 2D culture, in order to compare equitoxic doses. As expected, growth of the untreated spheroids (control) was observed. Indeed, in 3D culture, these spheroids show a very significant increase in their size over time with a diameter (d) of 313+/−0 μm at d0 against 785+/−6 μm at d14 (p<0.001). After treatment with PTX and NPC₂-PTX, the growth of the spheroids appears to be slowed down under certain conditions compared to the control (FIG. 14). At IC₅₀, no difference in size compared to the control was found at day 7 and day 14 for both the PTX and the NPC₂-PTX. Indeed, the diameters measured at day 14 are respectively 800+/−11 μm (p=0.10) for the PTX and 799+/−16 μm (p=0.23) for the NPC₂-PTX.; At 2.8 times IC₅₀, no difference was found on day 7 and day 14 for the PTX compared to the control.

Thus at day 14, the diameter measured after treatment with PTX is 757+/−22 μm (p=0.10). On the other hand, less growth is observed with the NPC₂-PTX, at day 7 and is confirmed on day 14 where the measured diameter of the spheroid is 368+/−22 μm (p<0.001).; At 4 times the IC₅₀, a very significant difference in growth compared to the control is found at day 7 and is confirmed at day 14 for both the PTX whose diameter measured on day 14 is 389+/−33 μm (p<0.001) and for NPC₂-PTX whose diameter on day 14 is equal to 316+/−12 μm (p<0.001); Thus, after treatment with NPC₂-PTX, from a concentration equal to 2.8 times IC₅₀ calculated in 2D culture, there is observed a major inhibition of the growth of the spheroid relative to the control, from d7 (p<0.001), which is confirmed 4 times the IC₅₀. It is demonstrated in 3D culture, on U-87-MG cells, an inhibition of the growth of spheroids after treatment with NPC₂-PTX greater than that observed with PTX alone (at 2D equitoxic doses).

At IC₅₀, however, there was no effect on the spheroids for both PTX and NPC₂-PTX. This lack of 3D activity may be due to a greater difficulty in the penetration of active molecules into spheroids than into cells in 2D culture.; (2) Subsequently, an Alamar Blue cell viability test was carried out on the spheroids. At 2.8 times IC₅₀, the NPC₂-PTX exhibited a significantly higher activity than the PTX at 7 days of treatment (p=0.0079) (FIG. 18A), as well as at 14 days (p=0.011) (FIG. 18B). At 4 times the IC₅₀, the inhibition of spheroid growth induced by the NPC₂-PTX is similar to that induced by the PTX at 7 days (p=0.98) (FIG. 15A) and at 14 days (p=0.30) (FIG. 18B). The results of the Alamar Blue test therefore confirm the growth inhibition observed by light microscopy and highlight a dose-dependent cytotoxic effect. Thus, at equitoxic doses measured in 2D culture, the NPC₂-PTX exhibit an activity superior to PTX in 3D culture. Indeed, at 2.8 times the IC₅₀, the PTX and NPC₂-PTX have a similar activity (70% inhibition of cell viability) in 2D. In contrast, in 3D, PTX induces only a 10% decrease in cell viability, while NPC₂-PTX induces a 60% inhibition of viability.

### Example 27: Study of Cytotoxicity in 3D Culture

Aim: to observe changes in size of the spheroids induced by the NPC₂-PTX, compared to the control, and to measure the cell survival rate on cells in 3D culture.

### 3D Cell Culture

Spheroids (cells in 3D culture) help to better represent the tumor environment. They are composed of necrotic cells in their center and proliferating cells in the periphery. The application of NPC₂-PTX on these spheroids allows them to be studied over a longer time compared to the 2D study. The GL261 cells are cultured in DMEM medium supplemented with 10% of FCS, 2 mM of L-glutamine, 100 U/ml of penicillin-streptomycin and 20% (m/v) of methylcellulose. The cells are seeded on a round bottom 96-well culture plate, at the concentration of 1000 cells/well, 72 h before treatment. The cells are maintained at 37° C. and 5% of CO 2 and 10 μl of fresh medium are added every two days to all the wells (controls and treated).

### Treatment

Cells are incubated in culture medium (control cells) or containing NPC₂ (76 and 153 μg/ml) or NPC₂-PTX (76 and 153 μg/ml NPC₂ equivalent, i.e.; 10 and 20 mM PTX equivalent). Before treatment and then every day, the spheroids are observed under an optical microscope coupled to a camera (Nikon ECLIPSE TS100), at magnification*4. The area is calculated from the ImageJ software at each time, at each concentration, and it is reported to the control=normalized area. The viability of the spheroids after 13 days of treatment is calculated with an Alamar Blue test. Twenty microliters of Alamar Blue are added to each well 13 days after treatment and the cells are incubated 24 h before the fluorescence measurement. The data are obtained by reading the fluorescence with POLARstar Omega (BMG LABETCH) after excitation at 584 nm and reading the emission at 620 nm. The percentage of viability is obtained by comparing the rate of fluorescence emitted by the reduced Alamar Blue reagent in the treated cells with the rate present in the untreated control cells, which is considered as a reference and equivalent to 100% of viability.

The standard deviation is calculated from 3 independent experiments.

### Results—FIGS. 19-21

Growth of untreated spheroids was observed (control) (FIG. 19). After treatment with NPC₂, the size of the spheroids was not significantly modified compared to the control. The size of the spheroids treated with NPC₂-PTX is significantly reduced compared to the control, in a time dependent manner (FIG. 19). Also, this effect is dose dependent with an effect significantly greater than 20 M compared to 10 μM (d10, difference <0.05). Subsequently, an Alamar Blue cell viability test was carried out on the spheroids. The viabilities are compared to those obtained by the control spheroids (=100%). After treatment 13 days with NPC₂ at 153 μg/ml, no difference in viability was found compared to the control. After 13 days treatment with NPC₂-PTX at 153 μg/ml equivalent, ie 20 M PTX equivalent, the NPC₂-PTX inhibits cell viability very significantly compared to NPC₂ (p<0.001).

The results of the Alamar Blue test therefore confirm the growth inhibition observed by light microscopy and highlight a dose-dependent cytotoxic effect. unmodified NPC₂ show safety on spheroids while NPC₂-PTX show dose- and time-dependent anticancer activity (FIG. 21)

### Example 28: Stability Study Under Storage Conditions (4° C.)

Aim: to determine the stability of the NPC₂-PTX bond in stock solution.

The purity of the NPC₂s dispersed in ultrapure water is determined by HPLC, in order to quantitatively determine the presence of free PTX which could have been released from its binding from the NPC₂-PTXs under storage conditions. The chromatographic separation of the PTX is carried out on a Phenomenex Kinetex XD-C18 column (2.1×100 mm, 2.6 μm). The isocratic elution is carried out with a mobile phase composed of 50% acetonitrile, 1 mM ammonium acetate and 0.05% formic acid, at a flow rate of 0.3 ml/min. The effluent is detected at 229 nm with a diode array detector. The standard PTX calibration curves (10 to 1000 nM) show a retention time of 3.3±0.2 min. A quantification limit of 10 nM has been determined.

### Results

Up to 8 weeks after solubilization of NPC₂-PTX, no specific peak of PTX is found in HPLC, meaning that the chemical ester bond between PTX and NPC₂ is stable in the mother solution for an extended time.

### Example 29: Stability Study in a Biological Medium at 37° C.

Aim: to determine the stability of the NPC₂-PTX bond under physiological conditions.

The NPC₂-PTX were diluted in complete EMEM medium to a final concentration of 10 μM PTX equivalent and stored at 37° C. At various times, from 15 minutes to 2 weeks, 100 μl of this solution was collected. A microliter of internal standard (docetaxel) at 1 mg/ml was added and mixed with 200 μl of 5% SDS, then 200 μl of dichloromethane. After centrifugation, the organic phase is preserved, and the liquid extraction is repeated twice on the aqueous phase. After evaporation of the organic phase, the solid residue is dissolved in 300 μl of mobile phase before HPLC assay. The extraction yield of the PTX is suitable, corresponding to 88.5±2.8% (n=4). Results

No specific PTX peak was detected above the limit of quantification (10 nM) at 15 min, 4 h, 24 h, 48 h, 1 week and 2 weeks, indicating the absence of premature release of PTX of its binding and indicating the stability of NPC₂-PTX.

### Example 30: Study of Stability in the Blood of Mice

Aim: to determine the stability of the NPC₂-PTX bond in the blood of mice

The amount of free PTX present in a sample of whole blood of C57BL/6 mice containing a concentration of 60 mg/l of NPC₂-PTX (equivalent to 6 mg/l of PTX) was determined by liquid-liquid extraction and HPLC assay after incubation for 48 hours at room temperature. The extraction of free PTX is carried out in the same way as from the culture medium.

### Results

After 48 h of incubation at room temperature, the concentration of free PTX in the blood was 0.64 mg/l, indicating a release of approximately 11% of the PTX load of NPC₂.

### Example 31: Determination of DMT after Single Intra-Cerebral Injection of NPC₂ and NPC₂-PTX

Aim: to assess the maximum tolerated dose (MTD) of NPC₂-PTX and NPC₂ during an intracerebral injection in healthy C57BU6 mice.

### Method

Two groups of healthy animals received an injection into the right striatum of particles dispersed in PBS, at doses of 11 mg/kg for NPC₂-PTX (n=4) and 20 mg/kg for NPC₂ (n=3). After anesthesia with a mixture of ketamine (100 mg/kg) and xylazine (10 mg/kg), the animals are placed on a stereotaxic frame and the hydration of the eyes is maintained by applying an eye gel. The skin of the skull is shaved, brushed with betadine, and incised, then the skull is pierced with a strawberry at the following coordinates from the bregma: +2 mm to the right and +1 mm anteriorly. The needle of a 25 μL Hamilton syringe is inserted 3 mm from the surface of the brain and left in place 3 minutes before the injection begins. The injection of a total volume of 20 μL is carried out with an electric syringe pump according to the following scheme: 3 μL in 15 minutes, then 5 μL in 10 minutes, and finally 12 μL in 15 minutes. The injection is followed by a pause time of 5 minutes before raising the needle, carried out at a rate of 0.5 mm per minute.

The wound is sutured, and the animals are monitored until awakening. The behavior of the animals is then observed for 14 days and their weight regularly measured.

### Results—FIG. 22

None of the animals treated with NPC₂-PTX or NPC₂ died or showed any abnormal clinical signs following treatment. In addition, the change in body weight remained normal in both groups for the 14 days following the intracerebral injection. These results demonstrate a satisfactory tolerance of NPC₂ and NPC₂-PTX in C57BL/6 mice at the respective doses of 20 mg/kg and 11 mg/kg during intracerebral administration.

1. An active nanoplatform which consists of:
a nanoplatform (NPC₂) comprising or consisting of carbon, hydrogen, oxygen, and nitrogen, in the form of primary amino group (s) and primary alkylamino group (s) of 1 to 10 carbons, in particular of primary amino group (s) and of primary ethylamino group (s),
an active antitumor molecule (M),
optionally a fluorophore (F), and
optionally an addressing agent (A),
and wherein
NPC₂ is bound by covalent bonds with M via a linker L,
said NPC₂ nanoplatform having the following properties:
the solubility of NPC₂ in an aqueous medium is 25 to 500 g/l
the Young's modulus of NPC₂ is from 1 to 4 GPa,
the density of NPC₂ is 1 to 3,
the dry size of NPC₂ is 10 to 40 nm,
the hydrodynamic diameter of NPC₂ is 10 to 150 nm,
NPC₂ is substantially amorphous,
said nanoplatform NPC₂ having a number of primary amine functions of surface grafting of 1.4 to 7 mmol per gram of nanoplatform, said active anti-tumor molecule M having a solubility in aqueous medium of less than 200 mg/l,
said active nanoplatform having a solubility in an aqueous medium of 5 to 500 g/l,
with the provisio that:
NPC₂ is substantially free of lipids, nucleic acids, proteins and peptides.

2. The active nanoplatform according to claim 1, wherein in which the NPC₂ nanoplatform is bound by covalent bonds with M via a linker chosen from the compounds of formula XI, 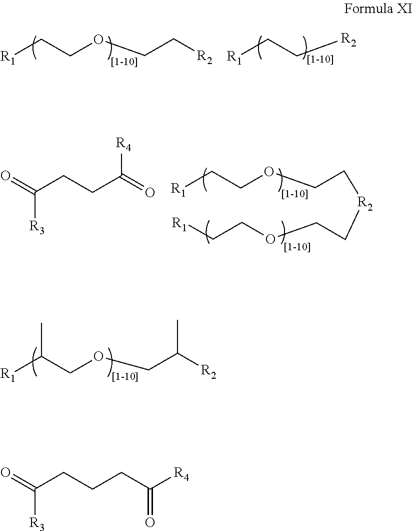
wherein:
R₁ and R₂ are independently chosen from: —NH—, —COO, —NHCO—, —O—, —OCO—, —NHCSNH—, —NHCONH— and —CO—NH—NH—CO—
R₃ is chosen from: —NH—; —O—
R₄ is chosen from: —O—, —NHNH—, NH—
in particular via a succinic linker.

3. The active nanoplatform according to claim 1, wherein:
said active nanoplatform comprises a fluorophore F and wherein NPC₂ is bound by covalent bonds with F, via an L_{F} linker, or
said active nanoplatform comprises an agent addressing A and wherein NPC₂ is bound by covalent bonds with A, via an L_{A} linker, or
wherein said active nanoplatform comprises a fluorophore F and an addressing agent A and wherein NPC₂ is bound by covalent bonds with F via an L_{F} linker and NPC₂ is bound by covalent bonds with A via an L_{A} linker.

4. The active nanoplatform according to claim 1, wherein said active nanoplatform is of Formula I 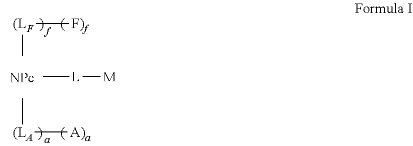
wherein:
NP_{C} represents the core of the NPC₂ nanoplatform without the grafting functions located on the surface, NPC₂ having the meaning of claim 1,
M, F and A having the meanings of claim 1,
f and a are integers independently from 0 or 1,
L_{F}, L and L_{A} represent the linkers linking, via covalent bonds, the core of the NP_{C} nanoplatform with compounds F, M and A of Formula II, Formula IIa and Formula IIb,
T_{F}Z_{F}—R_{F}-Q_{F})_{I}_{_{F}} Formula II
TZ—R-Q)_{I} Formula IIa
T_{A}Z_{A}—R_{A}-Q_{A})_{I}_{_{A}} Formula IIb
wherein:
T_{F}, T and T_{A} represent the grafting functions of the NPC₂ nanoplatform after their integration into L_{F}, L and L_{A} linkers,
Z_{F}, Z and Z_{A} represent the binding functions of the L_{F}, L and L_{A} linkers after their binding on the grafting functions T_{F}, T and T_{A},
R_{F}, R and R_{A} represent the functional chains of L_{F}, L and L_{A} linkers,
Q_{F}, Q and Q_{A} represent the binding functions of L_{F}, L and L_{A} linkers after their binding to the fluorophore, the active antitumor molecule and the addressing agent,
I, I_{F} and l_{A} are integers equal to or different from each other and are 0 or 1.

5. The active nanoplatform according to claim 1, wherein:
said active nanoplatform is of Formula III, 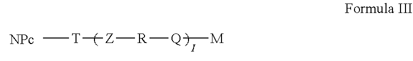
wherein:
NP_{C}, T, Z, R, Q, M and I have the meanings the meanings of claim 1
or wherein said active nanoplatform is of Formula IV, 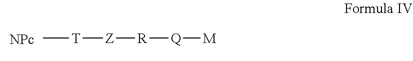
or wherein said active nanoplatform is of Formula V, 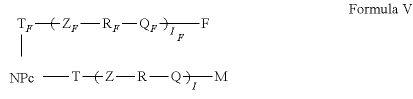
Wherein:
NP_{C}, T_{F}, T, Z_{F}, Z, R_{F}, R, Q_{F}, Q, l_{F}, I, F and M have the meanings of claim 1,
or wherein said active nanoplatform is of Formula VI, 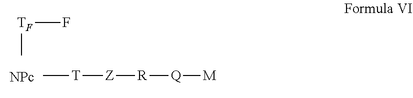
or wherein said active nanoplatform is of Formula VII, 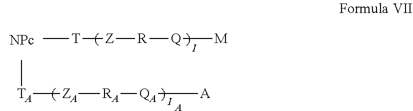
Wherein:
NP_{C}, T, T_{A}, Z, Z_{A}, R, R_{A}, Q, Q_{A}, I, I_{A}, A and M have the meanings of claim 1,
or wherein said active nanoplatform is of Formula VIII, 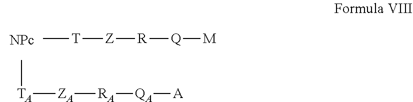
or wherein said active nanoplatform is of Formula IX, 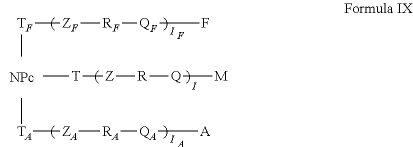
wherein:
NP_{C}, T, T_{F}, T_{A}, Z, Z_{F}, Z_{A}, R, R_{F}, R_{A}, Q, Q_{F}, Q_{A}, I, I_{F}, l_{A}, F, A and M have the meanings of claim 1,
or wherein said active nanoplatform is of Formula X,

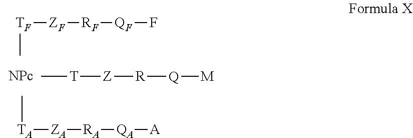

6. The active nanoplatform according to claim 1, wherein said active anti-tumor molecule M is chosen in the group of taxanes or in the group of anthracyclines.

7. The active nanoplatform according to claim 1, wherein said fluorophore F is chosen in the group consisting of Rhodamine B, Fluorescein, Lucifer Yellow cadaverine, the Alexa Fluor family and the NIR cyanine family.

8. The active nanoplatform according to claim 1, wherein said addressing agent is chosen in the group consisting of antibodies or vector peptides, in particular an antibody targeting EGF receptors, the RGD peptide or a LDLR targeting peptide.

9. The active nanoplatform according to claim 1, wherein said linkers L, L_{F} and L_{A} are chosen in the group consisting of the compounds of Formula XI, 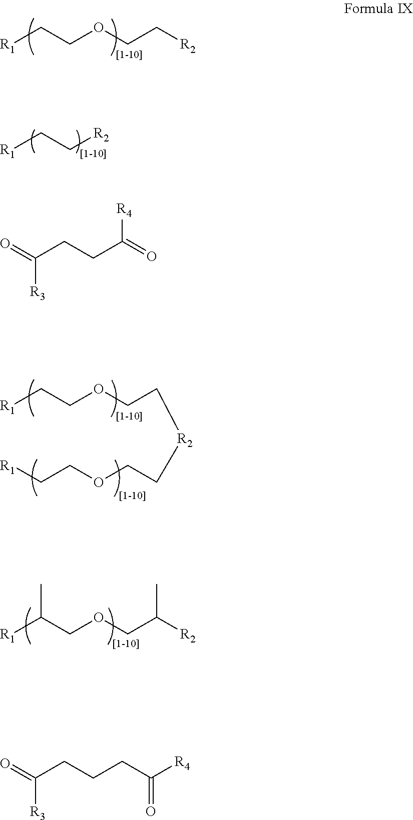
wherein:
R₁ and R₂ are independently chosen in the group consisting of: —NH—, —COO, —NHCO—, —O—, —OCO—, —NHCSNH—, —NHCONH— and —CO—NH—NH—CO—
R₃ is chosen in the group consisting of: —NH— and —O—
R₄ is chosen in the group consisting of: —O—, —NHNH—, and NH— In particular a succinic linker

10. The active nanoplatform according to claim 1, corresponding to Formula XII or to Formula XIIA, 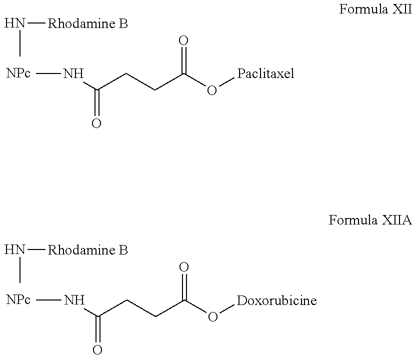
Wherein:
NP_{C} has the meaning of claim 1.

11. A process for the preparation of an active nanoplatform according to claim 1, comprising a step of grafting an active antitumor molecule,
by optionally bringing an NPC₂ nanoplatform into contact with a precursor L′ of said linker L, to obtain a nanoplatform bound by covalent bond to said linker L, followed
by bringing said nanoplatform bound by covalent bond to said linker L into contact with an active antitumor molecule M,
wherein NPC₂, L and M have the meanings of claim 1,
to obtain an active nanoplatform consisting of a nanoplatform bound by covalent bond to said active antitumor molecule.

12. The process for the preparation of an active nanoplatform according to claim 11, comprising the following steps:
a. a step of re-functionalizing a nanoplatform,
by bringing a nanoplatform into contact with an organic molecule of the α-ω diamino-alkane type of 1 to 10 carbon atoms, comprising two primary amine functions, in particular 1,2-ethylenediamine, in order to increase the rate of grafting comprising amine groups at the surface of said nanoplatform and thus obtaining a re-functionalized nanoplatform,
b. optionally a step of binding a fluorophore,
by optionally bringing said nanoplatform optionally re-functionalized into contact with precursor L′_{F} of said L_{F} linker, to obtain a nanoplatform re-functionalized and optionally bound to said L_{F} linker, followed
by optionally bringing said nanoplatform re-functionalized and optionally bound to said linker L_{F} into contact with a fluorophore F, in order to obtain a nanoplatform optionally re-functionalized and optionally bound to said fluorophore F,
c. a step of grafting an active anti-tumor molecule,
by optionally bringing said nanoplatform re-functionalized and optionally bound to said fluorophore F into contact with a precursor L′ of said linker L, in order to obtain a nanoplatform re-functionalized, optionally bound to said fluorophore F and bound by covalent bond to said linker L, followed
by bringing said nanoplatform re-functionalized, optionally bound to said fluorophore F and bound by covalent bond to said linker L into contact with an active antitumor molecule M, to obtain an active nanoplatform consisting of a nanoplatform bound by covalent bond to said active molecule antitumor M, refunctionalized and optionally bound to said fluorophore F,
d. optionally a step of linking an addressing agent,
by optionally bringing said nanoplatform bound by covalent bond to said active antitumor molecule M, refunctionalized and optionally bound to said fluorophore F into contact with a precursor L′_{A} of said linker L_{A}, to obtain a nanoplatform bound by covalent bond to said active molecule anti-tumor M, refunctionalized, optionally bound to said fluorophore F and optionally bound to said L_{A} linker, followed
by optionally bringing said nanoplatform bound by covalent bond to said active antitumor molecule M, refunctionalized, optionally bound to said fluorophore F and optionally bound to said L_{A} linker into contact with an addressing agent A,
wherein NPC₂, L_{F}, L, L_{A}, F, M and A have the meanings of claim 1,
to obtain an active nanoplatform consisting of a nanoplatform bound by covalent bond to said active antitumor molecule M, refunctionalized, optionally bound to said fluorophore F and optionally bound to said addressing agent A,
said optional addressing agent being a vector peptide.

13. The process for the preparation of an active nanoplatform according to claim 11, comprising the following steps:
a. a step of re-functionalizing a nanoplatform,
by bringing a nanoplatform into contact with an organic molecule of the α-ω diamino-alkane type of 1 to 10 carbon atoms, comprising two primary amine functions, in particular 1,2-ethylenediamine, in order to increase the rate of grafting comprising amine groups at the surface of said nanoplatform and thus obtaining a re-functionalized nanoplatform,
b. optionally a step of binding a fluorophore,
by bringing said re-functionalized nanoplatform into contact with succinic anhydride to obtain a re-functionalized nanoplatform by means of a succinic linker, followed
by bringing the nanoplatform refunctionalized by said succinic linker into contact with hydrazine to obtain a nanoplatform refunctionalized by a modified succinic linker, followed
by optionally bringing said nanoplatform refunctionalized by a succinic linker modified into contact with a fluorophore F, the quantity of fluorophore F being substoichiometric with respect to the number of succinic linkers activated, to obtain a nanoplatform refunctionalized by an activated succinic linker and optionally bound to said fluorophore F,
c. a step of linking an addressing agent,
by bringing said refunctionalized nanoplatform into contact with a modified succinic linker and optionally bound to said fluorophore F with an addressing agent A in order to obtain a refunctionalized nanoplatform optionally bound to said fluorophore F and bound to said addressing agent A,
d. a step of grafting an active anti-tumor molecule,
by optionally bringing said refunctionalized nanoplatform, optionally bound to said fluorophore F and bound to said addressing agent A into contact with a precursor L′ of said linker L, in order to obtain a refunctionalized nanoplatform, optionally bound to said fluorophore F, bound to said agent addressing and optionally bound by covalent bond to said linker L, followed
by bringing said refunctionalized nanoplatform into contact, optionally bound to said fluorophore F, bound to said addressing agent and optionally bound by covalent bond to said linker L with an active antitumor molecule M, to obtain an active nanoplatform consisting of a bound nanoplatform by covalent bond to said anti-tumor active molecule M, refunctionalized and optionally bound to said fluorophore F and bound to an addressing agent,
wherein NPC₂, L_{F}, L, L_{A}, F, M and A have the meanings of claim 1,
to obtain an active nanoplatform consisting of a nanoplatform bound by covalent bond to said active anti-tumor molecule M, refunctionalized, optionally bound to said fluorophore F and bound to said addressing agent A, said addressing agent being an antibody.

14. The process for the preparation of an active nanoplatform according to claim 11, said active nanoplatform being of Formula I, 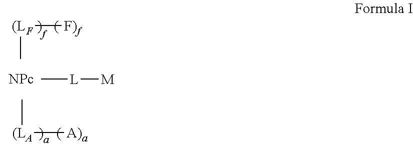
wherein:
NP_{C}, F, M, A, L, L_{F}, L_{A}, a and f have the meanings of claim 1,
said process comprises:
a. a refunctionalization step by bringing an NPC₁ nanoplatform of Formula a-b into contact 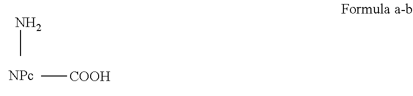
with an organic molecule of the type an organic molecule of the α-ω diamino-alkane type of 1 to 10 carbon atoms, comprising two primary amine functions, in particular 1,2-ethylenediamine, to obtain an NPC₂ nanoplatform of Formula b-b or of Formula B, 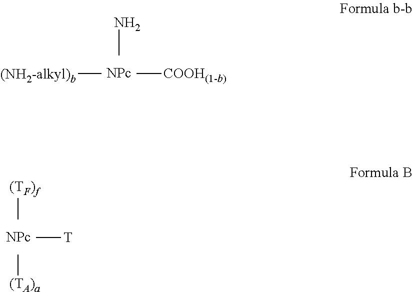
wherein:
b is a real number from 0 to 1 corresponding to the rate of re-functionalization of the COOH grafting functions in NH₂
the grafting functions T_{F}, T and T_{A} are chosen from the grafting functions of the nanoplatform of Formula b-b (Alkyl-NH₂, NH₂, COOH),
a and f are integers from 0 or 1, equal or different,
with the proviso that the number b is greater than 0.
b. optionally a step of binding a fluorophore by optionally contacting an L′_{F} linker precursor
with a NPC₂ nanoplatform of Formula B
to obtain a nanoplatform of Formula C 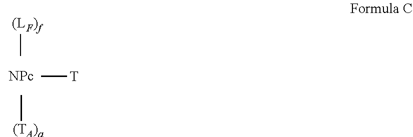
followed by bringing said nanoplatform of Formula C into contact with said fluorophore F to obtain a nanoplatform of Formula D, 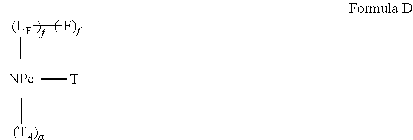
c. a step of grafting an active anti-tumor molecule by optionally contacting said nanoplatform of Formula D with a precursor of linker L′
to obtain a nanoplatform of Formula E, 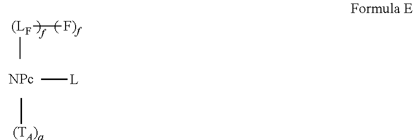
followed by bringing said nanoplatform of Formula E into contact with said active antitumor molecule M to obtain an active nanoplatform of Formula F, 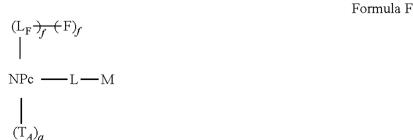
d. optionally a step of linking an addressing agent by optionally contacting said active nanoplatform of Formula F with a precursor of linker L′_{A}
to obtain an active nanoplatform of Formula G 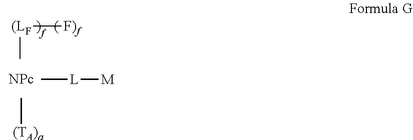
followed by bringing said active nanoplatform of Formula G into contact with said addressing agent A to obtain said active nanoplatform of Formula I.

15. The process for the preparation of an active nanoplatform according to claim 11, said active nanoplatform being of Formula XII, 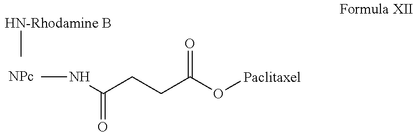
wherein:
NP_{C} has the meaning of claim 1,
said method comprising:
a. a step of synthesis of an NPC₁ nanoplatform of Formula a-b, 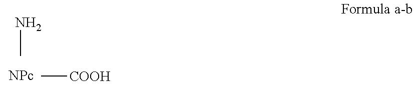
by bringing citric acid into contact with diethylenetriamine in water under microwaves with a power of 500 to 1000 W, in particular 600 W, for a time of 1 to 5 minutes, in particular 2 minutes, to obtain said nanoplatform of Formula ab,
b. a step of refunctionalizing said NPC₁ nanoplatform of Formula ab, by bringing said NPC₁ nanoplatform of Formula ab into contact with an excess of 1,2-ethylenediamine at a temperature of 100 to 180° C. for 2 to 24 hours, in particular 12 hours, to obtain a NPC₂ nanoplatform of Formula bb or Formula AE, 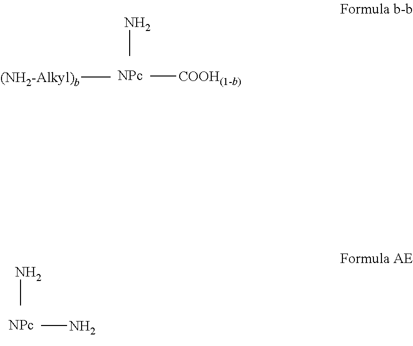
b has the meaning of claim 14
c. a step of linking a fluorophore by bringing a fluorophore F constituted by Rhodamine B into contact with said NPC₂ nanoplatform of Formula AE,
to obtain a nanoplatform of Formula AF 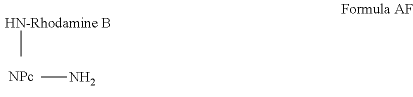
d. a step of grafting an active antitumor molecule by bringing said nanoplatform of Formula AF into contact with a linker precursor L′, succinic anhydride and a base, in particular sodium carbonate or diisopropylethylamine, to obtain the nanoplatform of Formula AG 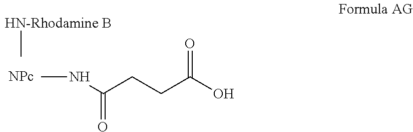
followed by bringing said nanoplatform of Formula AG into contact with said active antitumor molecule M, paclitaxel, in order to obtain said active nanoplatform of Formula XII.

16. An active nanoplatform according to claim 1, for use as a medicament.

17. A pharmaceutical composition comprising at least one active nanoplatform according to claim 1, wherein said active antitumor molecule M is chosen in the group consisting of taxanes, more particularly paclitaxel and docetaxel or from anthracyclines, more particularly epirubicin, pirarubicin, idarubicin, zorubicin, aclarubicin and doxorubicin and said active nanoplatform being in association with a pharmacologically acceptable excipient.

18. A method for treating cancers and brain tumors, more particularly glioblastoma and brain metastases originating from different primary tumors, comprising administering an effective amount of a nanoplatform according to claim 1, to a patient in need thereof.

19. The active nanoplatform according to claim 2, wherein:
said active nanoplatform comprises a fluorophore F and wherein NPC₂ is bound by covalent bonds with F, via an L_{F} linker, or
said active nanoplatform comprises an agent addressing A and wherein NPC₂ is bound by covalent bonds with A, via an L_{A} linker, or
wherein said active nanoplatform comprises a fluorophore F and an addressing agent A and wherein NPC₂ is bound by covalent bonds with F via an L_{F} linker and NPC₂ is bound by covalent bonds with A via an L_{A} linker.

20. The active nanoplatform according to claim 1, wherein said active nanoplatform is of Formula I 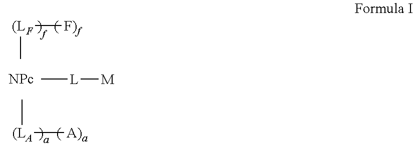
wherein:
NP_{C} represents the core of the NPC₂ nanoplatform without the grafting functions located on the surface, NPC₂ having the meaning of claim 1,
M, F and A having the meanings of claim 1,
f and a are integers independently from 0 or 1,
L_{F}, L and L_{A} represent the linkers linking, via covalent bonds, the core of the NP_{C} nanoplatform with compounds F, M and A of Formula II, Formula IIa and Formula IIb,
T_{F}Z_{F}—R_{F}-Q_{F})_{I}_{_{F}} Formula II
TZ—R-Q)_{I} Formula IIa
T_{A}Z_{A}—R_{A}-Q_{A})_{I}_{_{A}} Formula IIb
wherein:
T_{F}, T and T_{A} represent the grafting functions of the NPC₂ nanoplatform after their integration into L_{F}, L and L_{A} linkers,
Z_{F}, Z and Z_{A} represent the binding functions of the L_{F}, L and L_{A} linkers after their binding on the grafting functions T_{F}, T and T_{A},
R_{F}, R and R_{A} represent the functional chains of L_{F}, L and L_{A} linkers,
Q_{F}, Q and Q_{A} represent the binding functions of L_{F}, L and L_{A} linkers after their binding to the fluorophore, the active antitumor molecule and the addressing agent,
I, I_{F} and l_{A} are integers equal to or different from each other and are 0 or 1.
